(19) United States
(12) Patent Application Publication
(10) Pub. No.: US 2024/0209417 A1
(43) Pub. Date: Jun. 27, 2024

(54) SYSTEMS AND METHODS FOR NEXT GENERATION SEQUENCING UNIFORM PROBE DESIGN

(71) Applicant: Tempus Labs, Inc., Chicago, IL (US)

(72) Inventors: Richard Blidner, Glenview, IL (US); Hala Samir Kuttab-Boulos, Barrington, IL (US)

(21) Appl. No.: 18/556,838

(22) PCT Filed: Apr. 21, 2022
(86) PCT No.: PCT/US22/25854

§ 371 Date: Oct. 23, 2023

Related U.S. Application Data
(60) Provisional application No. 63/177,811, filed on Apr. 21, 2021.

(51) Int. Cl. C12Q 1/6811; C12Q 1/6874

(52) U.S. Cl. CPC C12Q 1/6811; C12Q 1/6874

(57) ABSTRACT

Systems and methods are provided for balancing a probe set for enriching a plurality of genomic loci. A nucleic acid probe set containing pools of nucleic acid probe species is obtained. Each probe species aligns to a different subsequence of a respective locus and includes proportions of a capture moiety conjugated version and a capture moiety-free version. Each probe species in a pool aligns to a portion of the genome that is at least 100 nucleotides away from any other probe species in the pool. Each pool in the probe set is separately analyzed against reference nucleic acid samples to obtain recovery rates and identify probe species that do not satisfy a minimum or a maximum recovery rate threshold. An adjusted version of a final design for the probe set is established by adjusting proportions of capture moiety conjugated and capture moiety-free versions for the identified probe species.

### CROSS REFERENCE TO RELATED APPLICATIONS

This application claims priority to U.S. Provisional Patent Application No. 63/177,811 filed Apr. 21, 2021, the content of which is hereby incorporated by reference, in its entirety, for all purposes.

### TECHNICAL FIELD

The present disclosure relates generally to designing efficient probes for use in next generation sequencing.

### BACKGROUND

One aspect of the design of next generation sequencing assays is the selection and concentration of probes used to identify specific regions of a genome.

In the prior art, one method of reducing probe concentration is to add the reverse complement of each over-performing probe, thereby effectively subtracting a certain percentage of such over-performing probes from an existing probe pool. Another method of setting probe concentration is to utilize an array-based platform. Some methods known in the prior art make use of probe sub-pools, which are formulated at known equimolar concentrations. This enables the modular use of sub-pools (e.g., each sub-pool is distinct and can be modified separately from the other sub-pools).

What is needed in the field are improved methods of altering probe concentrations to produce probe pools that are optimized for particular samples.

### SUMMARY

Given the background above, improved systems and methods are needed for improved probe design, in particular for use with targeted next-generation sequencing. Advantageously, the present disclosure provides solutions to these and other shortcomings in the art. For instance, in some embodiments, the systems and methods described herein leverage multiple methods of probe modification to improve the overall coverage rate of a set of probes. Additionally, in some embodiments, the systems and methods described herein improve the overall coverage rate of a set of probes for a plurality of genomic loci by balancing the coverage of each probe in the probe set across the plurality of loci. As discussed below in Example 2, in some implementations, balancing probe sets for enrichment of a plurality of loci improves the overall coverage rate by reducing the amount by which certain probes and/or subsequences of loci are overrepresented or underrepresented during analysis, such as sequencing analysis.

Accordingly, one aspect of the present disclosure provides a method for balancing a probe set for enriching a plurality of genomic loci, comprising obtaining a first iteration of a nucleic acid probe set comprising a plurality of nucleic acid probe species distributed in a first plurality of pools. The plurality of nucleic acid probe species comprises, for each respective locus in the plurality of loci, a respective sub-plurality of nucleic acid probe species, where each respective nucleic acid probe species in the respective sub-plurality of nucleic acid probe species aligns to a different subsequence of the respective locus. Each respective nucleic acid probe species is present in the first iteration of the nucleic acid probe set as (i) a respective first proportion of a non-nucleotidic capture moiety conjugated version of the respective nucleic acid probe species, and (ii) a respective second proportion of a capture moiety-free version of the respective nucleic acid probe species, and each nucleic acid probe species present in a respective pool, in the first plurality of pools, aligns to a portion of the genome that is at least 100 nucleotides away from any other portion of the genome that any other nucleic acid probe species present in a respective pool aligns with.

The method further includes analyzing the first iteration of the nucleic acid probe set against a first plurality of reference nucleic acid samples to obtain a corresponding recovery rate of each respective nucleic acid probe species in the plurality of nucleic acid probe species, where each respective pool in the first plurality of pools is analyzed in a separate reaction. Based on the corresponding recovery rate of each respective nucleic acid probe species, a first subset of the plurality of nucleic acid probe species that does not satisfy a minimum recovery rate threshold, and a second subset of the plurality of nucleic acid probe species that does not satisfy a maximum recovery rate threshold are identified.

The identification of the first and second subsets of the plurality of nucleic acid probe species is used to make a first adjustment to respective proportions of (i) non-nucleotidic capture moiety conjugated versions and (ii) capture moiety-free versions of the respective nucleic acid probe species in a final design for the nucleic acid probe set, thereby establishing a first adjusted version of the final design for the nucleic acid probe set, where the proportion of non-nucleotidic capture moiety conjugated versions of respective nucleic acid probe species in the first subset of the plurality of nucleic acid probe species are adjusted upwards in the final design for the nucleic acid probe set by the first adjustment, and the proportion of non-nucleotidic capture moiety conjugated versions of respective nucleic acid probe species in the second subset of the plurality of nucleic acid probe species are adjusted downwards in the final design for the nucleic acid probe set by the first adjustment.

In some embodiments, the method further includes obtaining a second iteration of the nucleic acid probe set comprising the plurality of nucleic acid probe species distributed in a second plurality of pools. Each respective nucleic acid probe species is present in the second iteration of the nucleic acid probe set as (i) a respective third proportion of a non-nucleotidic capture moiety conjugated version of the respective nucleic acid probe species, and (ii) a respective fourth proportion of a capture moiety-free version of the respective nucleic acid probe species, based on the first adjusted version of the final design for the nucleic acid probe set. Each nucleic acid probe species present in a respective pool, in the second plurality of pools, aligns to a portion of the genome that is at least 100 nucleotides away from any other portion of the genome that any other nucleic acid probe species present in a respective pool aligns with.

The second iteration of the nucleic acid probe set is analyzed against a second plurality of reference nucleic acid samples to obtain a corresponding recovery rate of each respective nucleic acid probe species in the plurality of nucleic acid probe species, where each respective pool in the second plurality of pools is analyzed in a separate reaction. Based on the corresponding recovery rate of each respective nucleic acid probe species, a third subset of the plurality of nucleic acid probe species that does not satisfy a minimum recovery rate threshold, and a fourth subset of the plurality of nucleic acid probe species that does not satisfy a maximum recovery rate threshold are identified. The identification of the third and fourth subsets of the plurality of nucleic acid probe species is used to make a second adjustment to respective proportions of (i) non-nucleotidic capture moiety conjugated versions and (ii) capture moiety-free versions of the respective nucleic acid probe species in the final design for the nucleic acid probe set, thereby establishing a second adjusted version of the final design for the nucleic acid probe set, where the proportion of non-nucleotidic capture moiety conjugated versions of respective nucleic acid probe species in the third subset of the plurality of nucleic acid probe species are adjusted upwards in the final design for the nucleic acid probe set by the second adjustment, and the proportion of non-nucleotidic capture moiety conjugated versions of respective nucleic acid probe species in the second subset of the plurality of nucleic acid probe species are adjusted downwards in the final design for the nucleic acid probe set by the first adjustment.

In one aspect, the disclosure provides a method for forming a nucleic acid probe set enriched for a plurality of loci within a genome. The method includes obtaining a first iteration of the nucleic acid probe set, where the first iteration of the nucleic acid probe set includes a plurality of nucleic acid probe species distributed in a first plurality of pools. The plurality of nucleic acid probe species includes, for each respective locus in at least a portion of the plurality of loci, a respective sub-plurality of nucleic acid probe species, where each respective nucleic acid probe species in the respective sub-plurality of nucleic acid probe species aligns to a different subsequence of the respective locus. Each respective nucleic acid probe species in the plurality of probe species is present in the first iteration of the nucleic acid probe set in a combination of a respective first proportion and second proportion that sums to a respective amount, where each nucleic acid probe species in the respective first proportion is a non-nucleotidic capture moiety conjugated version of the respective nucleic acid probe species and each nucleic acid probe species in the respective second proportion is a capture moiety-free version of the respective nucleic acid probe species. Each nucleic acid probe species present in a respective pool, in the first plurality of pools, aligns to a portion of the genome that is at least 50 nucleotides away from any other portion of the genome that any other nucleic acid probe species present in a respective pool aligns with.

The method also includes separately analyzing each respective pool in the first plurality of pools in the first iteration of the nucleic acid probe set against a first plurality of reference nucleic acid samples to obtain a corresponding recovery rate of each respective nucleic acid probe species in the plurality of nucleic acid probe species. The method then includes identifying, based on the corresponding recovery rate of each respective nucleic acid probe species, a first subset of the plurality of nucleic acid probe species that does not satisfy a minimum recovery rate threshold and a second subset of the plurality of nucleic acid probe species that does not satisfy a maximum recovery rate threshold. The method then includes adjusting each respective first proportion of each respective nucleic acid probe species in the first subset of the plurality of nucleic acid probe species and each respective first proportion of each respective nucleic acid probe species in the second subset of the plurality of nucleic acid probe species based on the identification of the first and second subsets of the plurality of nucleic acid probe species, thereby establishing a first adjusted version of the nucleic acid probe set.

In some embodiments, in the first adjusted version of the nucleic acid probe set, the respective first proportion of each respective nucleic acid probe species in the first subset of the plurality of nucleic acid probe species is at a higher proportion than the respective first proportion of each respective nucleic acid probe species in the plurality of nucleic acid probe species that satisfied the minimum recovery rate threshold and the respective first proportion of each respective nucleic acid probe species in the second subset of the plurality of nucleic acid probe species is at a lower proportion than the respective first proportion of each respective nucleic acid probe species in the plurality of nucleic acid probe species that satisfied the maximum recovery rate threshold.

As disclosed herein, any embodiment disclosed herein when applicable can be applied to any other aspect.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1 illustrates a block diagram of an example computing device, in accordance with some embodiments of the present disclosure.

FIG. 2 provides a flow chart of processes and features for determining an optimized set of probes for sequencing, in accordance with some embodiments of the present disclosure.

FIGS. 3A, 3B, 3C, and 3D collectively illustrate examples of how sets of probes may be modified through the use of sub-pools, in accordance with some embodiments of the present disclosure.

FIG. 4 illustrates an example of the improvement in the uniformity of sequencing coverage achieved using the optimized probe sets described herein, in accordance with some embodiments of the present disclosure.

FIG. 5 illustrates an example of the improvement in the uniformity of sequencing coverage achieved by selectively depleting over-expressed transcripts in a sample, in accordance with some embodiments of the present disclosure. An example of selective capture that can be used on RNA transcripts that are overexpressed, for example, one or more of mitochondrial genes, ribosomal genes, globin genes, or host genes can be depleted to help detect infectious pathogen sequences, etc. Overexpressed gene transcripts may be removed from the pool using selective capture to reduce concentration in the sequencing pool, in accordance with some embodiments of the present disclosure.

FIG. 6 illustrates an example of the improvement in the uniformity of sequencing coverage achieved using the optimized probe sets described herein, in accordance with some embodiments of the present disclosure. Variation in capture varies widely across the genome or target region. By balancing the capture labels on each probe the entire set can be tuned to more evenly distribute the capture efficiency across the genome or target region.

Sequencing depth is one method to measure probe performance. Alternative methods include measuring the number of reads associated with a target region or portion of a target region.

FIGS. 7A, 7B, and 7C collectively illustrate a block diagram of an example computing device for balancing a probe set for enriching a plurality of genomic loci, in accordance with some embodiments of the present disclosure.

FIGS. 8A, 8B, 8C, and 8D collectively illustrate an example method of balancing a probe set, in accordance with some embodiments of the present disclosure.

FIGS. 9A, 9B, and 9C collectively provide a flow chart of processes and features for balancing a probe set for enriching a plurality of genomic loci, in which optional features are indicated with dashed boxes, in accordance with some embodiments of the present disclosure.

FIGS. 10A, 10B, and 10C collectively show results from balancing a probe set based on pre-deduplicated recovery rates, in accordance with some embodiments of the present disclosure. FIG. 10A illustrates recovery rates for a first iteration of a first pool of probes in the probe set, determined from the number of raw sequence reads (i.e., pre-deduplication of sequence reads). FIG. 10B illustrates recovery rates for a second iteration of the first pool of probes in the probe set that was adjusted based on the pre-deduplication recovery rates illustrated in FIG. 10A, determined from the number of raw sequence reads (i.e., pre-deduplication of sequence reads) recovered using the second iteration of the first pool of probes. FIG. 10C illustrates recovery rates for a second iteration of the first pool of probes in the probe set that was adjusted based on the pre-deduplication recovery rates illustrated in FIG. 10A, determined from the number of deduplicated sequence reads recovered using the second iteration of the first pool of probes.

FIGS. 10D, 10E, and 10F collectively show results from balancing a probe set based on pre-deduplicated recovery rates, in accordance with some embodiments of the present disclosure. FIG. 10D illustrates recovery rates for a first iteration of a first pool of probes in the probe set, determined from the number of deduplicated sequence reads. FIG. 10E illustrates recovery rates for a second iteration of the first pool of probes in the probe set that was adjusted based on the post-deduplication recovery rates illustrated in FIG. 10D, determined from the number of raw sequence reads (i.e., pre-deduplication of sequence reads) recovered using the second iteration of the first pool of probes. FIG. 10F illustrates recovery rates for a second iteration of the first pool of probes in the probe set that was adjusted based on the post-deduplication recovery rates illustrated in FIG. 10D, determined from the number of deduplicated sequence reads recovered using the second iteration of the first pool of probes.

FIG. 11 provide a flow chart of processes and features for balancing a probe set for enriching a plurality of genomic loci, in which optional features are indicated with dashed boxes, in accordance with some embodiments of the present disclosure.

Like reference numerals refer to corresponding parts throughout the several views of the drawings.

### DETAILED DESCRIPTION

The methods described herein provide for optimizing a probe set for improved performance (e.g., with regards to a specific patient). In particular, the methods described herein provide for decreasing the effective concentration of one or more over-performing probes. In some embodiments, this is achieved by suppressing the capture rate of one or more over-performing probes by adjusting the ratio of labeled and unlabeled probe present in the set of probes used to assay a patient sample (e.g., for an individual probe, 30% of the probe molecules could be labeled with biotin while the remaining 70% of molecules are unlabeled). This suppression by capture method is novel to the art, and can be combined with other methods to increase or decrease the effective concentration of over- or under-performing probes (for example, adding locked nucleic acid/LNA or similar modifications to a portion of the probes, using hairpins, using interfering oligos, using HABA/4′-hydroxyazobenzene-2-carboxylic acid to interfere with streptavidin, using other probe immobilizers, interfering with hybridization kinetics, using other methods of adjusting the effective or functional concentration/molarity of the probe, etc.) in order to produce highly optimized probe sets with even capture rates (e.g., coverage). The systems and methods may also be combined with methods to reduce the amplification of certain RNA or DNA molecules during sequencing library generation (For example, blocking RNAs, knocking down RNA transcripts, and/or using siRNA, CRISPR, RNAse, etc. to reduce reads of certain nucleic acid molecules, for example, mRNA transcripts associated with highly expressed genes).

### Definitions

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “comprising,” or any variation thereof, when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the term “measure of central tendency” refers to a central or representative value for a distribution of values. Non-limiting examples of measures of central tendency include an arithmetic mean, weighted mean, midrange, midhinge, trimean, geometric mean, geometric median, Winsorized mean, median, and mode of the distribution of values.

As used herein, the terms “subject” or “patient” refers to any living or non-living human (e.g., a male human, female human, fetus, pregnant female, child, or the like). In some embodiments, a subject is a male or female of any stage (e.g., a man, a woman or a child).

As used herein, the terms “single nucleotide variant,” “SNV,” “single nucleotide polymorphism,” or “SNP” refer to a substitution of one nucleotide to a different nucleotide at a position (e.g., site) of a nucleotide sequence, for example, a sequence read from an individual. A substitution from a first nucleobase X to a second nucleobase Y may be denoted as “X>Y.” For example, a cytosine to thymine SNP may be denoted as “C>T.” The term “het-SNP” refers to a heterozygous SNP, where the genome is at least diploid and at least one—but not all—of the two or more homologous sequences exhibits the particular SNP. Similarly, a “hom-SNP” is a homologous SNP, where each homologous sequence of a polyploid genome has the same variant compared to the reference genome. As used herein, the term “structural variant” or “SV” refers to large (e.g., larger than 1 kb) regions of a genome that have undergone physical transformations such as inversions, insertions, deletions, or duplications (e.g., see review of human genome SVs by Spielmann et al., 2018, Nat Rev Genetics 19:453-467).

As used herein, the term ‘indel’ refers to insertion and/or deletion events of stretches of one or more nucleotides, either within a single gene locus or across multiple genes.

As used herein, the term “copy number variant,” “CNV,” or “copy number variation” refers to regions of a genome that are repeated. These may be categorized as short or long repeats, in regards to the number of nucleotides that are repeated over the genome regions. Long repeats typically refer to cases where entire genes, or large portions of a gene, are repeated one or more times.

As used herein, the term “mutation,” refers to a detectable change in the genetic material of one or more cells. In a particular example, one or more mutations can be found in, and can identify, cancer cells (e.g., driver and passenger mutations). A mutation can be transmitted from a parent cell to a daughter cell. A person having skill in the art will appreciate that a genetic mutation (e.g., a driver mutation) in a parent cell can induce additional, different mutations (e.g., passenger mutations) in a daughter cell. A mutation generally occurs in a nucleic acid. In a particular example, a mutation can be a detectable change in one or more deoxyribonucleic acids or fragments thereof. A mutation generally refers to nucleotides that are added, deleted, substituted for, inverted, or transposed to a new position in a nucleic acid. A mutation can be a spontaneous mutation or an experimentally induced mutation. A mutation in the sequence of a particular tissue is an example of a “tissue-specific allele.” For example, a tumor can have a mutation that results in an allele at a locus that does not occur in normal cells. Another example of a “tissue-specific allele” is a fetal-specific allele that occurs in the fetal tissue, but not the maternal tissue.

As used herein, the terms “sequencing,” “sequence determination,” and the like as used herein refers generally to any and all biochemical processes that may be used to determine the order of biological macromolecules such as nucleic acids or proteins. For example, sequencing data can include all or a portion of the nucleotide bases in a nucleic acid molecule such as an mRNA transcript or a genomic locus.

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, for example, using sequencing techniques or using probes, for example, in hybridization arrays or capture probes, or amplification techniques, such as the polymerase chain reaction (PCR) or linear amplification using a single primer or isothermal amplification.

As used herein, the term “read segment” or “read” refers to any nucleotide sequences including sequence reads obtained from an individual and/or nucleotide sequences derived from the initial sequence read from a sample obtained from an individual. For example, a read segment can refer to an aligned sequence read, a collapsed sequence read, or a stitched read. Furthermore, a read segment can refer to an individual nucleotide base, such as a single nucleotide variant.

As used herein, the term “read-depth,” “sequencing depth,” or “depth” refers to a total number of read segments from a sample obtained from an individual at a given position, region, or locus. The locus can be as small as a nucleotide, or as large as a chromosome arm, or as large as an entire genome. Sequencing depth can be expressed as “Yx”, for example, 50×, 100×, etc., where “Y” refers to the number of times a locus is covered with a sequence read. In some embodiments, the depth refers to the average sequencing depth across the genome, across the exome, or across a targeted sequencing panel. Sequencing depth can also be applied to multiple loci, the whole genome, in which case Y can refer to the mean number of times a loci or a haploid genome, a whole genome, or a whole exome, respectively, is sequenced. When a mean depth is quoted, the actual depth for different loci included in the dataset can span over a range of values. Ultra-deep sequencing can refer to at least 100× in sequencing depth at a locus.

As used herein, the term “reference exome” refers to any particular known, sequenced, or characterized exome, whether partial or complete, of any tissue from any organism or pathogen that may be used to reference identified sequences from a subject. Exemplary reference exomes used for human subjects, as well as many other organisms, are provided in the online GENCODE database hosted by the GENCODE consortium, for instance Release 29 (GRCh38.p12) of the human exome assembly.

As used herein, the term “reference genome” refers to any particular known, sequenced or characterized genome, whether partial or complete, of any organism or pathogen that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or pathogen, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes or genetic sequences. In some embodiments, a reference genome includes sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the term “sample” refers to a biological sample obtained from a subject (e.g., a patient). In some embodiments, a sample comprises blood, cfDNA, saliva, solid tissue, or FFPE tissue.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Example System Embodiments.

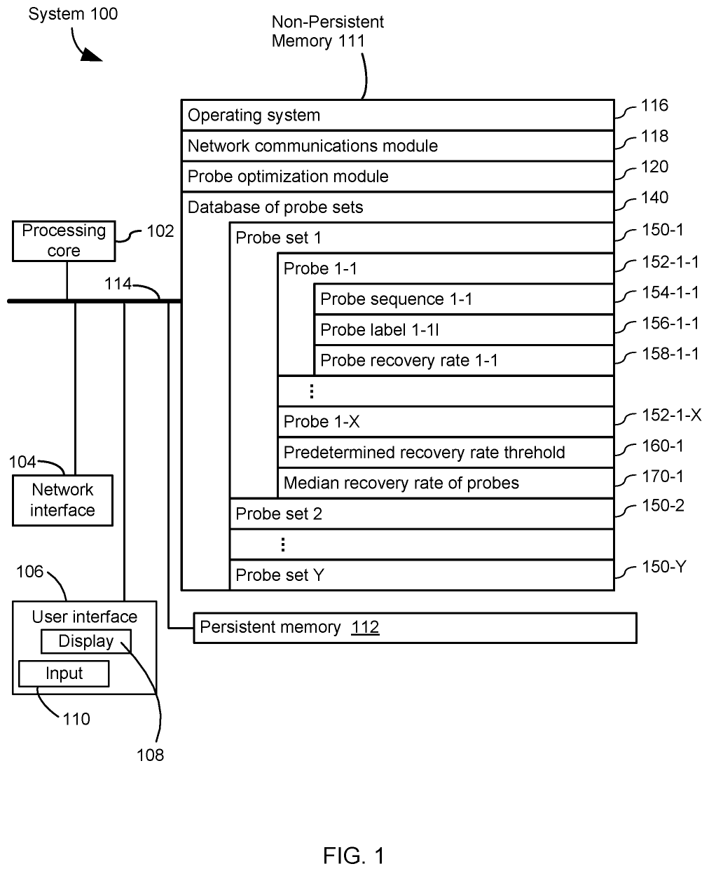

Now that an overview of some aspects of the present disclosure and some definitions used in the present disclosure have been provided, details of an exemplary system are described in conjunction with FIG. 1. FIG. 1 is a block diagram illustrating a system 100 in accordance with some implementations. The system 100 in some implementations includes one or more processing units CPU(s) 102 (also referred to as processors), one or more network interfaces 104, a user interface 106 including (optionally) a display 108 and an input system 110, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 100 with other devices and/or a communication network 104;
a probe optimization module 120 for determining an optimized set of probes for use against a sample (e.g., a nucleic acid sample from a patient); and
a database 140 of probe sets comprising, for each probe set 150, information for each probe 152 in a set of one or more probes including the respective sequence 154, optionally a respective label 156, and a respective recovery rate 158 resulting from assaying the respective probe against a sample library; each probe set 150 further includes a predetermined recovery rate threshold 160 (e.g., for determining which probes in the respective probe set could be optimized) and a median recovery rate of probe 170 across the respective probe set.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIG. 1 depicts a “system 100,” the figure is intended more as a functional description of the various features that may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules instead may be stored in persistent memory 112.

### Optimization of Probe Sets

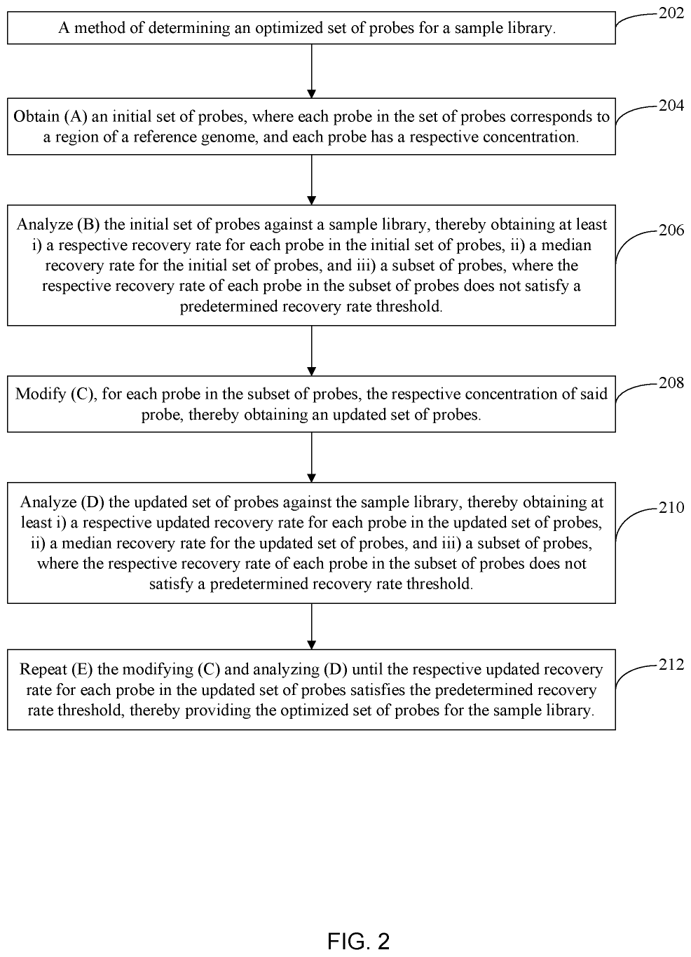

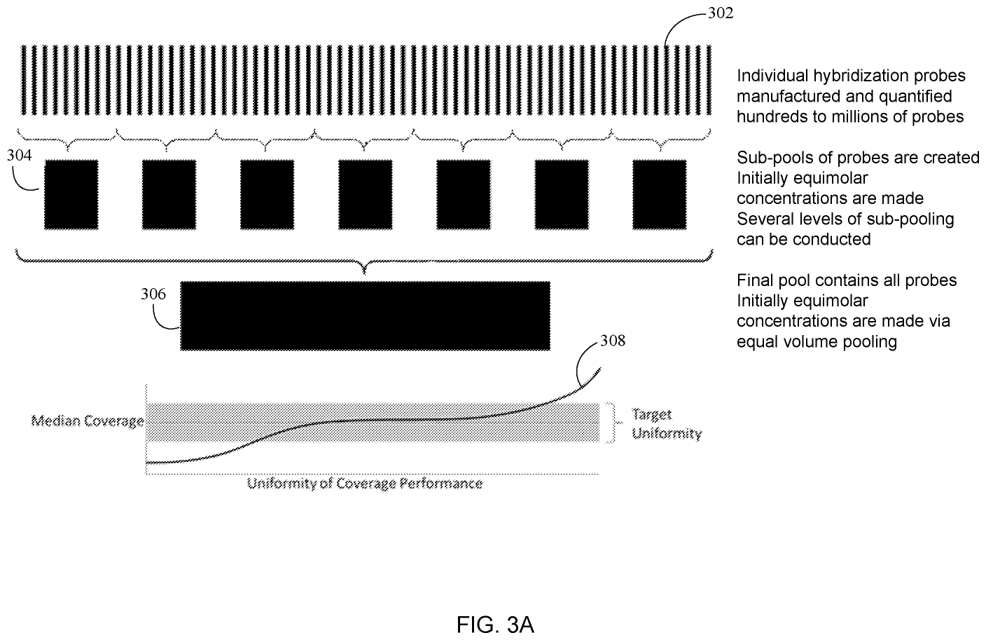

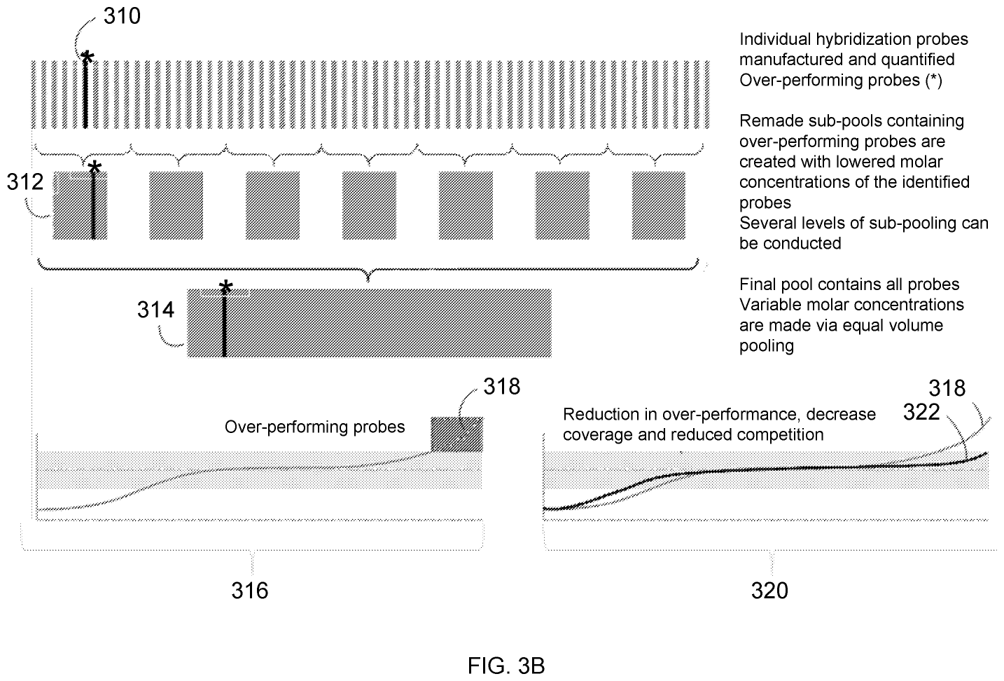

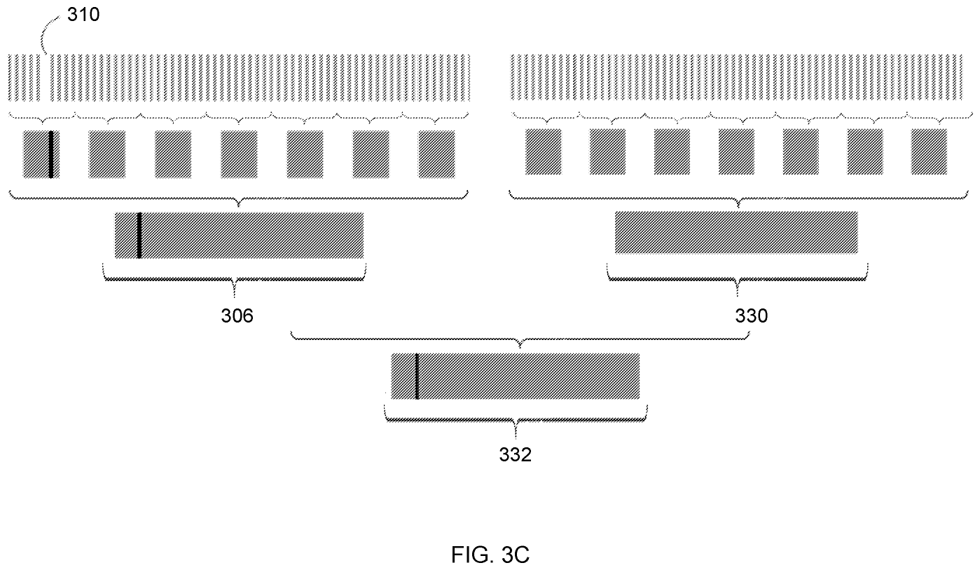

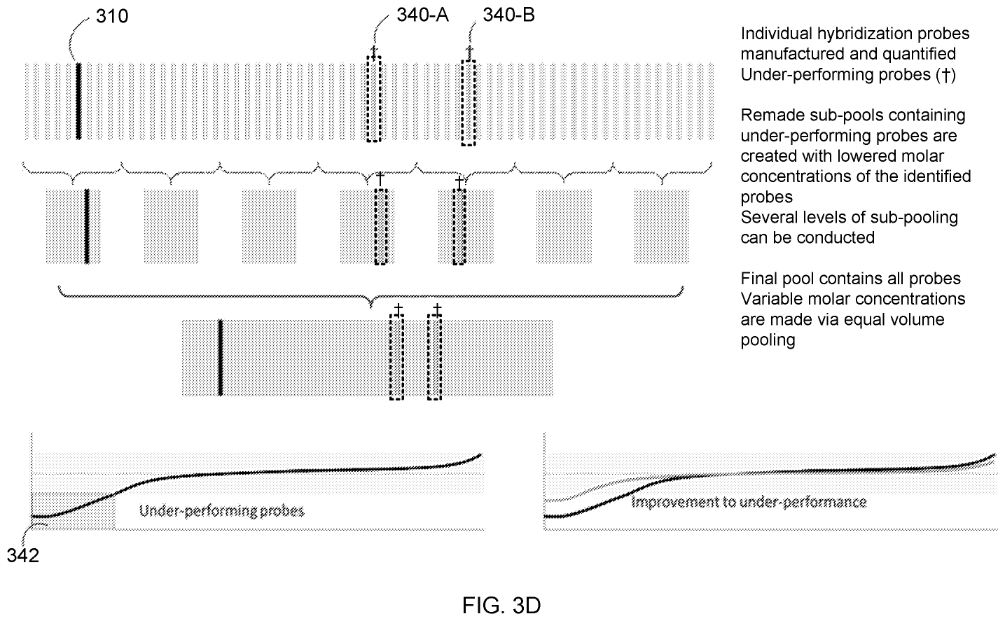

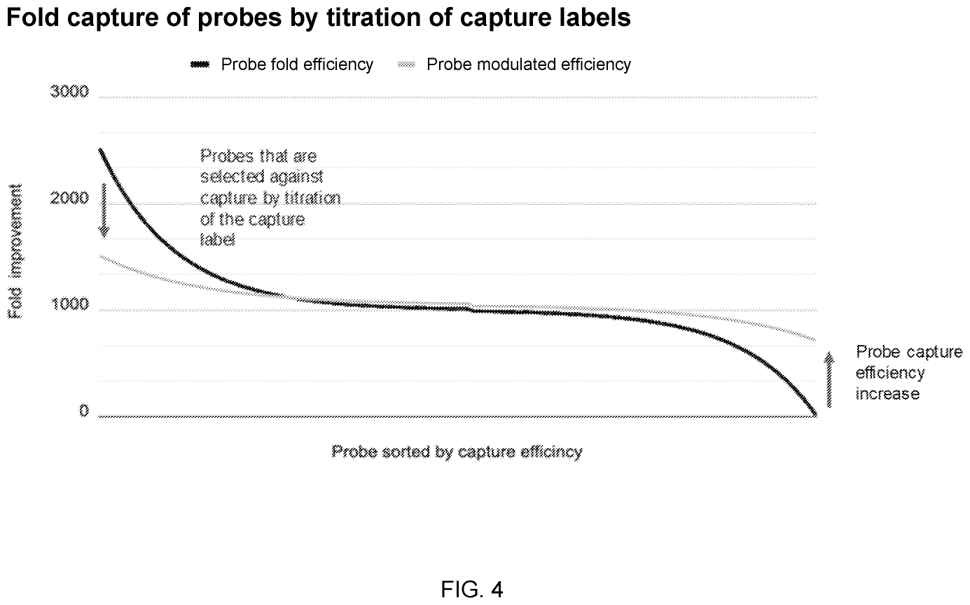

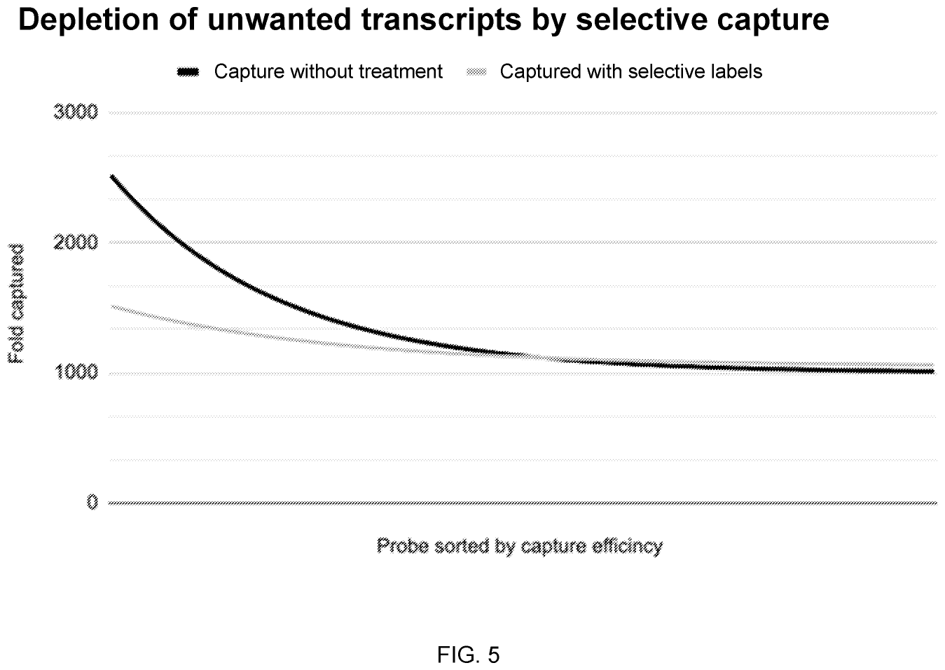

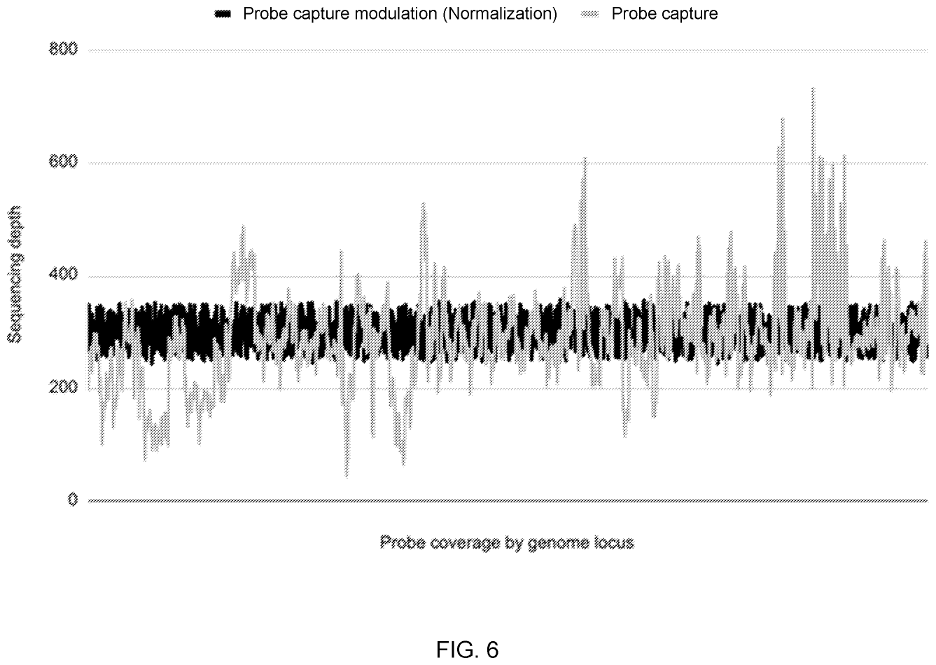

While a system in accordance with the present disclosure has been disclosed with reference to FIG. 1, methods in accordance with the present disclosure are now detailed below with reference to FIGS. 2 and 3A-3D. FIG. 2 provides an example outline of the methods described herein. FIGS. 3A-3D each provide illustrations of methods of probe set construction.

In some embodiments, the method comprises designing a genome assay by modifying the number and/or concentration of probes. In some embodiments, the steps of the method include 1) assaying the set of probes against a sample (e.g., a single patient sample, a reference sample, a collection of samples, etc.), 2) identifying probes with higher or lower recovery rates than the median recovery rate of the set of probes, 3) reducing the concentration of probes with a higher recovery rate than the median recovery rate and/or increasing the concentration of probes with a lower recovery rate than the median recovery rate, and 4) assaying the updated set of probes against the same or a substantially similar sample.

In some embodiments, the method proceeds as outlined in FIG. 2 and as described below.

Block 202. Referring to block 202, in some embodiments, the method determines an optimized set of probes for enriching a sample library (e.g., or sample libraries) preparatory to sequencing. In some embodiments, the sample library is for a single patient. In some embodiments, the sample library is for a plurality of patients. In some embodiments, the sample library is an exome panel (e.g., a backbone).

Block 204. Referring to block 204, in some embodiments, the method proceeds, by obtaining an initial set of probes, where each probe in the initial set of probes corresponds to a region of a reference genome or reference exome, and each probe has a respective concentration (e.g., molar concentration). In some embodiments, the initial set of probes is for sequencing the sample library with a predetermined mean read depth.

In some embodiments, each probe in the initial set of probes is present at a same concentration (e.g., the probes are present in equimolar concentration). In some embodiments, one or more probes in the set of probes are present in a different concentration (e.g., the molar concentration of one or more probes is varied).

In some embodiments, a whole exome backbone is used as the reference exome, and the set of probes comprises a plurality of probes that are present at a first probe concentration (e.g., to obtain a predetermined read depth), and at least one spike-in probe (e.g., for one or more specific targets) that are each present at a higher concentration than the first probe concentration (e.g., to obtain a higher read depth). In some embodiments, the first probe concentration is 0 (e.g., there are no probes other than the at least one spike-in probes present in the set of probes).

In some embodiments, the set of probes comprises i) a first subset of probes used to sequence the exome (e.g., the “backbone”), where each probe in the first subset of probes has a read depth of 75×, and ii) at least one spike-in probe with a read depth higher than 75×. In some embodiments, the higher read depth comprises at least 100×, at least 125×, at least 150×, at least 200×, at least 250×, at least 300×, at least 400×, at least 450×, at least 500×, or at least 550×.

In some embodiments, the at least one spike-in probes are targeted for sequencing loci associated with inherited cancer risks. In some embodiments, the at least one spike-in probes are to identify copy number variants, indels, and/or other mutations at particular loci. In some embodiments, each spike-in probe has a different read depth. In some embodiments, each probe in a probe set is associated with a specific cancer sub-type (e.g., each probe serves to help identify subjects that may have or be predisposed to have a particular cancer sub-type). In some embodiments, the optimized probe set targets specific areas of a reference genome (e.g., intron regions, exon region, immunology regions, or regions associated with susceptibility to or infection from a virus, bacteria, or other pathogen).

Block 206. Referring to block 206, in some embodiments, the method continues by analyzing the set of probes against a sample library, thereby obtaining at least i) a respective recovery rate (e.g., coverage) for each probe in the set of probes, ii) a median recovery rate (e.g., median coverage) for the set of probes, and iii) a subset of probes, where the respective recovery rate of each probe in the subset of probes does not satisfy a predetermined recovery rate threshold.

For example, as shown in FIG. 3A a plurality of probes 302 are combined into one or more sub-pools 304 of probes. These sub-pools 304 are then combined into a final set 306 of probes. The use of sub-pools enables finer tuning of the concentration of the different probes. In some embodiments, equal amounts of each sub-pool are combined to produce the final probe set. In some embodiments, one or more sub-pools are added at differing amounts to produce the final probe set. In some embodiments, equal amounts of each probe are present in each sub-pool and then also in the final probe set. In some embodiments, equal amounts of each probe are present in each sub-pool, but differing amounts of each sub-pool are combined to produce the final probe set. In some embodiments, one or more probes are present in the sub-pools at differing amounts.

Block 208. Referring to block 208, in some embodiments, the method continues by modifying, for each probe in the subset of probes, the respective concentration of said probe, thereby updating the set of probes. In some embodiments, modifying the concentration of one or more probes in the initial probe set comprises reducing the effective concentration of the one or more probes in the updated set of probes.

After assaying the final probe set against a sample library (e.g., a patient sample), the coverage (e.g., recovery rate) 308 for each probe is determined, and a median coverage rate can be calculated. In some embodiments, there is a target level of coverage for each probe (e.g., a tolerance of either over- or under-coverage). Over- and/or under-performing probes can then be identified from this first assay based on whether the respective recovery rate for each probe is above or below a predetermined threshold from the median coverage rate.

In some embodiments, each probe in the set of probes includes an attached label (e.g., each probe in the initial set of probes is biotinylated). See e.g., Miyazato et al. 2016 Scientific Reports 6, 28324. In some embodiments, each probe in the initial set of probes is unlabeled.

In some embodiments the attached label can be selectively captured from solution. The attached moiety can be a mixture of selective moieties that affect the capture or selection of the probe. Where by attached labels can be modulated bind and hold or interfere with binding or lack of binding, modulation of the kinetics of binding different probes with attach labels with different affinities. Binding moieties are not limited in scope of association; these could be covalent bonds, ionic bonding, polar covalent bonds, vander waal forces, hydrogen bonding, or electrostatic forces. These attached labels could include chemical alterations that affect the binding strength, alterations to the binding conditions, or alterations to the kinetics of the binding. Binding moieties could be modulated in concentration or type to affect selection of the desired probe. A plurality of binding moieties could be employed to modulate the effective capture of different groups of probes. The binding moieties could also be absent on the probe to modulate the effective population captured. Attached labels could also include a chemical cleavage group to modulate the effective capture of the probes. Examples of binding moieties include but are not limited to biotin: streptavidin, biotin: avidin, biotin:haba:streptavidin, antibody: antigen, antibody: antibody, covalent chemical linkage (ex. click chemistry).

In some embodiments binding moieties can be attached to a solid support, chemically modified linkers or in solution. Attachment labels can be attached to probes terminal groups or on the internal structure of the probe.

Block 210. Referring to block 210, in some embodiments, the method proceeds by analyzing the updated set of probes against the sample library, thereby obtaining at least i) a respective updated recovery rate for each probe in the updated set of probes, ii) a median recovery rate for the updated set of probes, and iii) a subset of probes, where the respective recovery rate of each probe in the subset of probes does not satisfy a predetermined recovery rate threshold.

In some embodiments, decreasing the concentration of over-performing probes comprises simply altering the total concentration of over-performing probes in the final set of probes. In some embodiments, the concentration of over-performing probes can be effectively decreased by decreasing the concentration of labeled over-performing probe. In embodiments where the initial set of probes includes unlabeled probes, the concentration of each over-performing probe can be corrected (e.g., adjusted so that all probes satisfy a predefined recovery rate threshold) by adding labeled (e.g., biotinylated) versions of each over-performing probe in proportion with labeled amounts of other probes in the probe set (e.g., to achieve even capture rates for each probe in the probe set). In some embodiments, the concentration of one or more over-performing probes can be reduced by reducing the percentage of over-performing probes that are biotinylated (e.g., by remaking each respective sub-pool that includes an over-performing probe).

For example, as shown in FIG. 3B, one or more over-performing probes 310 are identified (e.g., these are those probes with coverage rates 318 that are higher than the tolerated range around the median coverage rate, as identified in the results from the first assay 316 of the set of probes against a sample). In some embodiments, each sub-pool (e.g., 312) including an over-performing probe can be remade to result in a lower concentration of said probe (e.g., each said sub-pool is reformulated to adjust the individual molarity of one or more probes). This enables reuse of the one or more sub-pools that do not include over-performing probes (e.g., sub-pools that do not include over-performing probes do not need to be remade).

In some embodiments, the effective concentration of over-performing probes is reduced proportional to the detected recovery rate. In some embodiments, as shown in FIG. 3C, the effective concentration of one or more over-performing probes (e.g., 310) is reduced by adding the initial set of probes (e.g., 306) to a completely remade set of probes (e.g., 330) where the one or more over-performing probes have been excluded. This results in a final set of probes 332 where the concentration of one or more over-performing probes has been reduced based on the relative amounts of each of the component probe sets 306 and 330. For example, the effective concentration of each over-performing probe is reduced by at least 10%, by at least 20%, by at least 30%, by at least 40%, by at least 50%, by at least 60%, by at least 70%, by at least 80%, or by at least 90%.

In some embodiments, the effective concentration of one or more over-performing probes is reduced through suppression by competition. For example, in embodiments where the probes are labeled, the ratio of labeled to unlabeled probes can be altered (e.g., by reformulating one or more sub-pools that contain over-performing probes with unlabeled versions of said probes). In the art, such suppression is typically performed by adding a reverse complement of an over-performing probe to the set of probes; this reverse complement sequence then competes with the over-performing probe for hybridization with the target in the library. Such methods may add complexity to the hybridization with patient sample. In particular, reverse complement sequences may interact with other probes in the probe set. Altering the labeled to unlabeled ratio of particular probes may have less of an effect on the function of the probe set. Further, the percentage of labeled probe may be directly proportional to the percentage of captured target, making this method more tunable and sensitive than previous methods in the art.

Block 212. Referring to block 212, in some embodiments, the method repeats the modifying and analyzing from blocks 208 and 210, respectively, until the respective updated recovery rate for each probe in the updated set of probes satisfies the predetermined recovery rate threshold, thereby providing the optimized set of probes for the sample library (e.g., the method reruns the modified assay). For example, the coverage of each probe in the updated probe set is quantified again in light of the alterations to the updated probe set. In some embodiments, probe performance is reevaluated after each adjustment of effective probe concentration (e.g., after each one of the steps taken to alter effective probe concentrations).

The remade final probe set, which is produced by combining the initial sub-pools and one or more remade sub-pools, can in some embodiments be assayed again against the sample library (e.g., see 320 in FIG. 3B). As can be seen in FIG. 3B by comparing the coverage rates of the original set of probes 324 with the coverage rates of the updated set of probes 322, the reduction in concentration of over-performing probes can result in reduced coverage of the previously over-performing probes.

In some embodiments, the concentration of under-performing probes can be increased. Similar to the modulation of over-performing probes, one or more under-performing probes (e.g., those probes with capture rates 342 below the median capture rate) are identified as shown in FIG. 3D. In some embodiments, one or more sub-pools including one or more under-performing probes (e.g., 340-A and 340-B) can be reformulated to adjust the individual molarity of said under-performing probes.

Alternatively, similarly to FIG. 3C as described above, a second probe set (e.g., 330) is, in some embodiments, produced with either an increased molarity of under-performing probes or a decreased molarity of over-performing probes. By combining the redesigned probe set 330 with the first probe set 306 the concentration of under-performing probes can thus be increased.

In some embodiments, either under- or over-performing probes can be redesigned (e.g., by altering respective probe sequences) to alter binding affinities (e.g., to reduce the binding affinity of over-performing probes and/or to increase the binding affinity of under-performing probes).

In some embodiments, the method serves to optimize a probe set for a specific patient or a group of patients having a common characteristic (for example, a cohort of patients having the same cancer type or having the same variant). In such embodiments, the method proceeds by obtaining an initial set of probes; assaying the initial probe set against a sample of a specific patient; modifying the (effective) concentration of one or more selected probes to reduce the number of either over- or under-performing probes for the specific patient's sample; and rerunning the assay with the updated probe set. In such embodiments, as with other embodiments described herein, the modification and reanalysis steps are repeated as necessary until an optimal concentration of probes is achieved across a selected gene set. Such embodiments may be particularly useful for patients that will require multiple analyses (e.g., over time to monitor a health condition). In such circumstances, upon receipt of a subsequent sample from the specific patient, the assay can be rerun with the optimal concentration of probe sets. This aids in achieving standardized results for each patient and can help more accurately identify changes in a patient's results, leading to improved patient care and outcomes.

In some embodiments, where the probe set is optimized to a respective patient, the initial probe set is used to identify one or more nucleic acid (e.g., DNA or RNA) variants corresponding to said patient. In such embodiments, the initial probe set is then optimized using any method described herein to increase the effective concentration of probes that map to regions of interest (e.g., loci including an identified variant specific to the patient). In some embodiments, the concentration of probes that do not map to regions of interest (e.g., the negative backbone) is suppressed.

In some embodiments, optimizing a probe set for a specific cancer subtype requires using a sample library comprising one or more subject samples, where each subject has the specific cancer subtype.

In some embodiments, a method is provided for designing a uniform probe set. The method includes obtaining an initial set of probes, where each probe in the set of probes corresponds to a region of a reference genome, and each probe has a respective concentration. The method also includes analyzing the initial set of probes against a sample library, thereby obtaining at least i) a respective recovery rate for each probe in the initial set of probes, ii) a median recovery rate for the initial set of probes, and iii) a subset of probes, where the respective recovery rate of each probe in the subset of probes does not satisfy a predetermined recovery rate threshold. The method also includes modifying, for each probe in the subset of probes, the respective concentration of said probe, thereby obtaining an updated set of probes. The method also includes analyzing the updated set of probes against the sample library, thereby obtaining at least i) a respective updated recovery rate for each probe in the updated set of probes, ii) a median recovery rate for the updated set of probes, and iii) a subset of probes, where the respective recovery rate of each probe in the subset of probes does not satisfy a predetermined recovery rate threshold. The method then, optionally, includes repeating the modifying and analyzing until the respective updated recovery rate for each probe in the updated set of probes satisfies the predetermined recovery rate threshold, thereby providing the optimized set of probes for the sample library.

The embodiments described herein can be combined or used in any sequence as necessary to provide an optimized probe set suitable for a specific patient or for a particular assay (e.g., to assay for a mutation, specific cancer type, or other disease).

### Improved Probe Sets

In some embodiments, the present disclosure provides improved probe sets that facilitate a more uniform nucleic acid capture and/or more uniform sequencing depth across one or more target regions of a genome. The advantageous properties of the probe sets described herein are derived, at least in part, by separately tuning the percentage of individual probe species that are conjugated to a capture moiety, such as biotin. In this fashion, by increasing the conjugation percentage of an under-performing probe species (i.e., a probe species that aligns to a genomic sequence that is represented, on average, at a much lower sequencing depth than other genomic sequences following nucleic acid capture), relative to the conjugation percentage of other probe species, the resulting probe set facilitates a more uniform sequencing depth for the entire probe set, e.g., by increasing the sequencing depth for the genomic sequence aligning to the under-performing probe species.

For example, in some embodiments, an optimized probe set composition is provided. The composition includes a first set of nucleic acid probes for determining a genomic characteristic (e.g., a single nucleotide variant (SNV), an indel, a copy number variation (CNV), a pseudogene, a CG-rich region, an AT-rich region, a genetic rearrangement, a splice variant, a gene expression level, aneuploidy, or chromosomal trisomy) of a first target region in a genome (e.g., an short genomic sequence, an exon, and intron, a plurality of contiguous exons, a plurality of contiguous exons and introns, a gene, a cluster of genes, tens to hundreds of contiguous kilobases of a chromosome, a chromosome arm, or an entire chromosome) of a subject.

The first set of nucleic acid probes includes a first plurality of nucleic acid probe species. Each respective nucleic acid probe species (e.g., all nucleic acid probes that align to the same subsequence of the target region) in the first plurality of nucleic acid probe species aligns to a different subsequence of the first target region of a reference genome for the species of the subject. For instance, in some embodiments, the first set of nucleic acid probes tile (e.g., overlapping or non-overlapping tiling) a genomic region, such as a gene. Thus, the nucleic acid probes in the set of probes bind to different subsequences of the genomic region.

As used herein, a “nucleic acid probe species” refers to all nucleic acid probes in a composition that align to the same or substantially the same genomic sequence (e.g., the first 150 nucleotides of a particular exon of a gene). Generally, all probes of a particular nucleic acid probe species will have the same nucleotide sequence. However, in some embodiments, a particular probe of nucleic acid probe species may have one or a small number of nucleotide variations relative to other probes within the nucleic acid probe species. For instance, in some embodiments, different probes of a first nucleic acid probe species may include either an A or a G (or any other combination of bases) at a particular position (e.g., nucleotide 78 of the probe). Regardless, two probes that differ by one or a small number of nucleotide variants still belong to the same nucleic acid probe species because they align to the same position in the genome. Similarly, it can be envisioned that, in some embodiments, a probe in a particular nucleic acid probe species may be one or a small number of nucleotides longer or shorter than other probes in the particular nucleic acid probe species. Similarly, it can be envisioned that, in some embodiments, a probe in a particular nucleic acid probe species may be shifted by one or a small number of nucleotides relative to the sequence of other probes in the particular nucleic acid probe species. For instance, in some embodiments, a first probe of a particular nucleic acid probe species may align to nucleotides 1-150 of an exon, while a second probe of the particular nucleic acid probe species may align to nucleotides 3-152 of the same exon. Regardless, two probes that are shifted by two nucleotides still belong to the same nucleic acid probe species because they align to the essentially the same position in the genome. Similarly, probes in a particular nucleic acid probe species may be differently conjugated to a chemical moiety. For instance, a first probe aligning to a particular genomic subsequence that is not chemically linked to a capture moiety (e.g., biotin) and a second probe aligning to the same particular genomic subsequence that is chemically linked to a capture moiety (e.g., biotin) still belong to the same nucleotide probe species because they align to the same position in the genome.

The composition includes, for each respective nucleic acid probe species in the first plurality of nucleic acid probe species, a first amount of a first version of the respective nucleic acid probe species that is conjugated to a capture moiety (e.g., biotin) and a second amount of a second version of the respective nucleic acid probe species that is not conjugated to a capture moiety. That is, a certain percentage of the probes that constitute the first nucleic acid probe species are conjugated to a capture moiety. Generally, the percentage of conjugated probes ranges from about 1% to about 100%, based upon how well the probe performs in a plurality of reference nucleic acid capture and sequencing assays (e.g., a training or diagnostic cohort of assays meant to establish a baseline performance for particular probe species). As such, when the genomic subsequence that the nucleic acid probe species aligns to is over-represented, on average, in the sequencing results of the reference assays (in the training set), a smaller percentage of that nucleic acid probe species will be conjugated to the capture moiety in the composition, e.g., to reduce the representation of the corresponding genomic sequence in the sequencing results. Likewise, when the genomic subsequence that the nucleic acid probe species aligns to is under-represented, on average, in the sequencing results of the reference assays (in the training set), a greater percentage of that nucleic acid probe species will be conjugated to the capture moiety in the composition, e.g., to increase the representation of the corresponding genomic sequence in the sequencing results. In this fashion, the improved probe set compositions described herein can be tuned to provide more uniform sequence coverage across of a genomic region and/or across multiple genomic regions (e.g., across multiple genes in a targeted panel, an entire exosome, or an entire genome). In some embodiments, this also allows for tuning sequencing coverage across one or more genomic regions without varying the molar concentration of particular nucleic acid probe sequences, which prevents certain pull-down biases caused by using different molar concentrations for different probes.

As such, within the composition there is a first ratio (e.g., a first percentage), for a first respective nucleic acid probe species in the first plurality of the nucleic acid probe species that aligns to a first subsequence of the first target region, of (i) the first amount of the first version of the first respective nucleic acid probe species to (ii) the second amount of the second version of the first respective nucleic acid probe species. For instance, 45% of the first nucleic acid probe species are conjugated to biotin. Similarly, within the composition, there is a second ratio (e.g., a second percentage), for a second respective nucleic acid probe species in the first plurality of the nucleic acid probe species that aligns to a second subsequence of the first target region, of (i) the first amount of the first version of the second respective nucleic acid probe species to (ii) the second amount of the second version of the second respective nucleic acid probe species. For instance, 60% of the second nucleic acid probe species are conjugated to biotin. Accordingly, the first ratio is different from the second ratio. That is, the percentage of probes aligning to one subsequence that are conjugated is different from the percentage of probes aligning to a different subsequence that are conjugated.

In some embodiments, the concentration of the first respective nucleic acid probe species in the first plurality of nucleic acid probe species is equal to the concentration of the second respective nucleic acid probe species in the first plurality of nucleic acid probe species. In some embodiments, the concentration of each respective nucleic acid probe species in the first set of nucleic acid probes is equal in the composition. That is, in some embodiments, each probe species corresponding to a target region (e.g., all probes used to tile a gene, a smaller genomic region, or a larger genomic region) is included in a nucleic acid capture and sequence assay at the same concentration. However, the percentage of each probe that is conjugated to a capture moiety differs, e.g., to account for differences in the performance of each capture probe. In this fashion, artifacts caused by biases resulting from using different concentrations of different probes are avoided.

As such, the improved probe compositions provided herein are tuned to improve the uniformity of sequence coverage across the target region. Accordingly, in some embodiments, when the composition is used in a reference nucleic acid capture and sequencing assay, the assay outputs an equal number of raw sequencing reads of the first subsequence of the first target region and the second subsequence of the first target region. The reference nucleic acid capture and sequencing assay refers to the particular assay, or a substantially similar assay, that was used to tune the conjugation percentages for the probe set composition. That is, in some embodiments, when the improved probe set compositions described herein are under the same assay conditions that were used to establish a baseline performance for nucleic acid probe species in the composition, the tuned compositions provide a more uniform sequence coverage for two or more (e.g., at least 10%, 15%, 25%, 30%, 40%, 50%, 60%, 70%, 80%, 90%, 95%, 99%, or 100%) of the genomic subsequences within the target region. In some embodiments, the sequence coverage for the two or more subsequences is within a 25%. In some embodiments, the sequence coverage for the two or more subsequences is within a 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.1%, or smaller range.

In some embodiments, the range of the first distribution becomes at least 5% more uniform across the gene, gene panel, target region, expression panels, whole or targeted exome, or whole genome in raw sequencing reads. In some embodiments, the range of the first distribution becomes at least 10%, 15%, 20%, 25%, 30%, 35%, 40%, 45%, 50%, 60%, 70%, 80%, 90%, 100%, 200%, 300%, or more uniform across the gene, gene panel, target region, expression panels, whole or targeted exome, or whole genome in raw sequencing reads

Similarly, in some embodiments, when the composition is used in a reference nucleic acid capture and sequencing assay, the resulting sequence coverage between two or more (e.g., at least 10%, 15%, 25%, 30%, 40%, 50%, 60%, 70%, 80%, 90%, 95%, 99%, or 100%) of the genomic subsequences within the target region is improved by at least 25%, relative to the uniformity of the sequence coverage obtained when all of the probes are conjugated to the capture moiety at a same level (e.g., 100% or 50%). In some embodiments, the resulting sequence coverage between two or more of the genomic subsequences within the target region is improved by at least 30%, 40%, 50%, 60%, 70%, 80%, 90%, 100%, 150%, 200%, 250%, 300%, 400%, 500%, 600%, 700%, 800%, 900%, or more.

According, in some embodiments, when the composition is used in a first reference nucleic acid capture and sequencing assay, the difference between (i) the number of raw sequencing reads output for the first subsequence of the first target region and (ii) the number of raw sequencing reads output for the second subsequence of the first target region (e.g., the variance in sequence coverage between the subsequences) is less than the difference between (iii) the number of raw sequencing reads output for the first subsequence of the first target region in a second reference nucleic acid capture and sequencing assay and (iv) the number of raw sequencing reads output for the second subsequence of the first target region in the second reference nucleic acid capture and sequencing assay, when the first reference nucleic acid capture and sequencing assay and the second reference nucleic acid capture and sequencing assay are performed using the same methodology, the second reference nucleic acid capture and sequencing assay is performed with a second composition including the first respective nucleic acid probe species and the second respective probe species, and in the second composition, the percentage of the first respective nucleic acid probe species that are conjugated to the capture moiety and the percentage of the second respective nucleic acid probe species that are conjugated to the capture moiety are the same.

In some embodiments, the difference between (i) the number of raw sequencing reads output for the first subsequence of the first target region and (ii) the number of raw sequencing reads output for the second subsequence of the first target region is at least 25% less than the difference between (iii) the number of raw sequencing reads output for the first subsequence of the first target region in the second reference nucleic acid capture and sequencing assay and (iv) the number of raw sequencing reads output for the second subsequence of the first target region in the second reference nucleic acid capture and sequencing assay. In some embodiments, the difference in the variance in the first reference assay is at least 30%, 40%, 50%, 60%, 70%, 80%, 90%, 100%, 150%, 200%, 250%, 300%, 400%, 500%, 600%, 700%, 800%, or 900 less than the variance in the second reference assay.

In some embodiments, when the composition is used in a reference nucleic acid capture and sequencing assay, the assay outputs for each respective nucleic acid probe species in the first plurality of nucleic acid probe species a corresponding number of raw sequence reads, thereby forming a first distribution of numbers of raw sequence reads for the respective subsequences of the first target region that align with a respective nucleic acid probe species in the first set of nucleic acid probes, and the range of the first distribution is less than 250% of the median of the distribution. In some embodiments, the range of the first distribution is less than 50% percent of the median of the distribution. In some embodiments, the range of the first distribution is less than 300%, 200%, 150%, 100%, 75%, 50%, 25%, or 10% percent of the median of the distribution.

Similarly, in some embodiments, when the composition is used in a reference nucleic acid capture and sequencing assay, the assay outputs for each respective nucleic acid probe species in the first plurality of nucleic acid probe species a corresponding number of raw sequence reads, thereby forming a first distribution of numbers of raw sequence reads for the respective subsequences of the first target region that align with a respective nucleic acid probe species in the first set of nucleic acid probes, and the first distribution has a fold-80 score of less than 1.5. As used herein, a “fold-80 score” is the fold of additional sequencing required to ensure that 80% of the target bases achieve the mean coverage. The lower the on-target rate, or the higher the fold-80 score, the greater the non-uniformity in sequence coverage across the target region. Accordingly, in some embodiments, the first distribution has a fold-80 score of less than 2, 1.9, 1.8, 1.75, 1.7, 1.6, 1.5, 1.4, 1.3, 1.25, 1.2, 1.15, 1.1, or 1.05.

In some embodiments, when the composition is used in a reference nucleic acid capture and sequencing assay, the assay outputs for each respective nucleic acid probe species in the first plurality of nucleic acid probe species a corresponding number of raw sequence reads, thereby forming a first distribution of numbers of raw sequence reads for the respective subsequences of the first target region that align with a respective nucleic acid probe species in the first set of nucleic acid probes, and the range of the first distribution is less than the range of a second distribution. The second distribution is determined by using a second composition in the reference nucleic acid capture and sequencing assay to output, for each respective nucleic acid probe species in the first plurality of nucleic acid probe species, a corresponding number of raw sequence reads, thereby forming the second distribution of numbers of raw sequence reads for the respective subsequences of the first target region that align with a respective nucleic acid probe species in the first set of nucleic acid probes, where in the second composition, the percentage of each respective nucleic acid probe species in the first plurality of nucleic acid probe species that are conjugated to the capture moiety is the same. In some embodiments, the range of the first distribution is at least 50% less than the range of the second distribution. In some embodiments, the range of the first distribution is at least 25%, 30%, 40%, 50%, 60%, 70%, 80%, 90%, 100%, 150%, 200%, 250%, 300%, 400%, 500%, 600%, 700%, 800%, or 900%, less than the range of the second distribution. In some embodiments, the fold-80 score of the first distribution is at least 50% less than the fold-80 score of the second distribution. In some embodiments, the fold-80 score of the first distribution is at least 25%, 30%, 40%, 50%, 60%, 70%, 80%, 90%, 100%, 150%, 200%, 250%, 300%, 400%, 500%, 600%, 700%, 800%, or 900 less than the fold-80 score of the second distribution.

In some embodiments, the first plurality of nucleic acid probe species is at least 10 nucleic acid probe species. In some embodiments, the first plurality of nucleic acid probe species is at least 5, 10, 15, 20, 25, 30, 40, 50, 75, 100, 200, 300, 400, 500, 750, 1000, 2500, 5000, 10,000, or more nucleic acid probe species.

In some embodiments, the first target region is a nucleotide, a portion of an intron, a portion of an exon, an intron, an exon, a subset of contiguous exons for a gene, a subset of contiguous exons and introns for a gene, a gene, a portion of a chromosome, an arm of a chromosome, or an entire chromosome.

In some embodiments, the first target region is a gene selected from the group consisting of BRCA1, BRCA2, a CYP gene, CYP2D, a PMS2 pseudogene, a PMSCL pseudogene, DMD, MET, TP53, ALK, IGF1, TLR9, FLT3, and a TCR/BCR gene.

In some embodiments, the capture moiety is biotin. In some embodiments, the capture moiety can be chemically modified to bind and hold or interfere with binding or lack of binding. Modulation of the kinetics of binding different probes with attach labels can be achieved with different affinities. Binding moieties are not limited in scope of association. In some embodiments, these could be covalent bonds, ionic bonding, polar covalent bonds, vander waal forces, hydrogen bonding, or electrostatic forces. These attached labels could include chemical alterations that affect the binding strength, alterations to the binding conditions, or alterations to the kinetics of the binding. Binding moieties could be modulated in concentration or type to affect selection of the desired probe. A plurality of binding moieties could be employed to modulate the effective capture of different groups of probes. The binding moieties could also be absent on the probe to modulate the effective population captured. Attached labels could also include a chemical cleavage group to modulate the effective capture of the probes. Examples of binding moieties include but are not limited to biotin: streptavidin, biotin: avidin, biotin:haba:streptavidin, antibody: antigen, antibody: antibody, covalent chemical linkage (e.g., click chemistry).

In some embodiments, the optimized probe composition also includes a second set of nucleic acid probes for identifying a genomic characteristic of a second target region in the genome of the subject. The second set of nucleic acid probes includes a second plurality of nucleic acid probe species. Each respective nucleic acid probe species in the second plurality of nucleic acid probe species aligns to a different subsequence of the second target region of the reference genome for the species of the subject. Accordingly, the composition includes, for each respective nucleic acid probe species in the second plurality of nucleic acid probe species, a first amount of a first version of the respective nucleic acid probe species that is conjugated to the capture moiety and a second amount of a second version of the respective nucleic acid probe species that is not conjugated to a capture moiety. As such, within the composition, there is a third ratio, for a first respective nucleic acid probe species in the second plurality of the nucleic acid probe species that aligns to a first subsequence of the second target region, of (i) the first amount of the first version of the first respective nucleic acid probe species to (ii) the second amount of the second version of the first respective nucleic acid probe species. Similarly, within the composition, there is a fourth ratio, for a second respective nucleic acid probe species in the second plurality of the nucleic acid probe species that aligns to a second subsequence of the second target region, of (i) the first amount of the first version of the second respective nucleic acid probe species to (ii) the second amount of the second version of the second respective nucleic acid probe species. Because the conjugation of the probe species is tuned to account for differences in probe efficiencies, the third ratio is different from the fourth ratio.

In some embodiments, the concentration of the first respective nucleic acid probe species in the second plurality of nucleic acid probe species is equal to the concentration of the second respective nucleic acid probe species in the second plurality of nucleic acid probe species. In some embodiments, the concentration of each respective nucleic acid probe species in the second set of nucleic acid probes is equal in the composition. That is, in some embodiments, each probe species corresponding to a target region (e.g., all probes used to tile a gene, a smaller genomic region, or a larger genomic region) is included in a nucleic acid capture and sequence assay at the same concentration. However, the percentage of each probe that is conjugated to a capture moiety differs, e.g., to account for differences in the performance of each capture probe. In this fashion, artifacts caused by biases resulting from using different concentrations of different probes are avoided.

In some embodiments, the concentration of the first respective nucleic acid probe species in the second plurality of nucleic acid probe species is equal to the concentration of the first respective nucleic acid probe species in the first plurality of nucleic acid probe species. In some embodiments, the concentration of each respective nucleic acid probe species in the second set of nucleic acid probes is equal to the concentration of each respective nucleic acid probe species in the first set of nucleic acid probes in the composition. That is, in some embodiments, the concentrations of probes to two or more different genomic regions (e.g., two or more genes in a targeted gene panel, two or more genes in a whole exosome, or two or more genomic regions in a whole genome) are the same within the composition. In some embodiments, all of the probes in the composition are at the same concentration.

As described above with reference to the first set of nucleic acid probes, in some embodiments, when the composition is used in a reference nucleic acid capture and sequencing assay, the assay outputs an equal number of raw sequencing reads of the first subsequence of the second target region and the second subsequence of the second target region.

In some embodiments, the first ratio is different from the third ratio and the fourth ratio. That is, in some embodiments, the percentage of conjugated probes for a probe species in the first set of probes is different from the ratio of conjugated probes for two or more of the probe sequences in the second set of probes. In some embodiments, the second ratio is different from the third ratio and the fourth ratio.

In some embodiments, when the composition is used in a reference nucleic acid capture and sequencing assay, the assay outputs an equal number of raw sequencing reads of the first subsequence of the first target region and the first subsequence of the second target region.

In some embodiments, the concentration of each respective nucleic acid probe species in the second set of nucleic acid probes is equal in the composition.

In some embodiments, when the composition is used in a reference nucleic acid capture and sequencing assay, the assay outputs for each respective nucleic acid probe species in the second plurality of nucleic acid probe species a corresponding number of raw sequence reads, thereby forming a second distribution of numbers of raw sequence reads for the respective subsequences of the second target region that align with a respective nucleic acid probe species in the second set of nucleic acid probes, and the range of the second distribution is less than 250% of the median of the distribution. In some embodiments, the range of the second distribution is less than 50% percent of the median of the distribution. In some embodiments, the range of the second distribution is less than 300%, 200%, 150%, 100%, 75%, 50%, 25%, or 10% percent of the median of the distribution.

In some embodiments, when the composition is used in a reference nucleic acid capture and sequencing assay, the assay outputs for each respective nucleic acid probe species in the second plurality of nucleic acid probe species a corresponding number of raw sequence reads, thereby forming a second distribution of numbers of raw sequence reads for the respective subsequences of the second target region that align with a respective nucleic acid probe species in the second set of nucleic acid probes, and the second distribution has a fold-80 score of less than 1.5. In in some embodiments, the second distribution has a fold-80 score of less than 2, 1.9, 1.8, 1.75, 1.7, 1.6, 1.5, 1.4, 1.3, 1.25, 1.2, 1.15, 1.1, or 1.05.

In some embodiments, the second plurality of nucleic acid probe species is at least 10 nucleic acid probe species. Ins some embodiments, the second plurality of nucleic acid probe species is at least 5, 10, 15, 20, 25, 30, 40, 50, 75, 100, 200, 300, 400, 500, 750, 1000, 2500, 5000, 10,000, or more nucleic acid probe species.

In some embodiments, the first target region is a gene selected from BRCA1, BRCA2, a CYP gene, CYP2D, a PMS2 pseudogene, a PMSCL pseudogene, DMD, MET, TP53, ALK, IGF1, TLR9, FLT3, and a TCR/BCR gene.

In some embodiments, a method is provided for determining a genomic characteristic of a subject. The method includes contacting a sample comprising nucleic acids from the subject with an optimized probe composition as described herein. The method also includes recovering a portion of the nucleic acids using an agent that binds to the capture moiety, and sequencing the recovered portion of the nucleic acids, thereby identifying a genomic characteristic of the subject.

In some embodiments, the genomic characteristic includes a single nucleotide variant (SNV), an indel, a copy number variation (CNV), a pseudogene, a CG-rich region, an AT-rich region, a genetic rearrangement, a splice variant, a gene expression level, aneuploidy, or a chromosomal trisomy.

In some embodiments, the nucleic acids from the subject are obtained from a liquid biological sample from the subject. In some embodiments, the liquid biological sample is a blood sample or a blood plasma sample from the subject. In some embodiments, the nucleic acids from the subject are obtained from a solid biological sample from the subject. In some embodiments, the solid biological sample is a tumor sample or a normal tissue sample from the subject.

In some embodiments, the nucleic acids include mRNA or cDNA generated from mRNA, and the method also includes, prior to contacting the sample with the composition, selectively removing a portion of the mRNA or cDNA from a first gene that is represented in the sample at a level that is greater than the representation of at least 50% of the genes represented in the sample. In some embodiments, the first gene is represented in the sample at a level that is greater than the representation of at least 75% of the genes represented in the sample. In some embodiments, the first gene is represented in the sample at a level that is greater than the representation of at least 55%, 60%, 65%, 70%, 75%, 80%, 85%, 90%, or 95% of the genes represented in the sample.

In some embodiments, a method is provided for determining a genomic characteristic of a subject. The method includes identifying a first genomic characteristic of the subject from a first sample including nucleic acids from the subject by: contacting the first sample comprising nucleic acids from the subject with a first optimized probe composition as described herein, recovering a portion of the nucleic acids from the first sample using an agent that binds to the capture moiety, and sequencing the portion of the nucleic acids recovered from the first sample. The method includes identifying a second genomic characteristic of the subject from a second sample comprising nucleic acids from the subject by: contacting the second sample comprising nucleic acids from the subject with a second optimized probe composition as described herein, recovering a portion of the nucleic acids from the second sample using an agent that binds to the capture moiety, and sequencing the portion of the nucleic acids recovered from the second sample. The first set of nucleic acid probes in the first composition and the first set of nucleic acid probes in the second composition align to the same target region of the reference genome for the species of the subject. The first respective nucleic acid probe species in the first plurality of the nucleic acid probe species in the first composition and the first respective nucleic acid probe species in the first plurality of the nucleic acid probe species in the second composition align to the same subsequence of the same target region. The first ratio for the first respective nucleic acid probe species in the first plurality of the nucleic acid probe species in the first composition is different than the first ratio for the first respective nucleic acid probe species in the first plurality of the nucleic acid probe species in the second composition.

In some embodiments, the nucleic acids in the first sample are obtained from a biological sample from a first tissue in the subject and the nucleic acids in the second sample are obtained from a biological sample obtained from a second tissue in the subject. In some embodiments, the nucleic acids in the first sample are obtained from a solid biological sample from the subject and the nucleic acids in the second sample are obtained from a liquid biological sample from the subject. In some embodiments, the solid biological sample is a tumor sample or a normal tissue sample from the subject. In some embodiments, the liquid biological sample is a blood sample or a blood plasma sample from the subject. In some embodiments, the nucleic acids in the first sample are DNA and the nucleic acids in the second sample are RNA.

In some embodiments, the nucleic acids in the first sample represent a whole exome from the subject and the nucleic acids in the second sample represent a targeted panel of nucleic acid sequences from the subject.

### SPECIFIC EMBODIMENTS

The following clauses describe specific embodiments of the disclosure.
Clause 1. A composition comprising a first set of nucleic acid probes for determining a genomic characteristic of a first target region in a genome of a subject, wherein the first set of nucleic acid probes comprises a first plurality of nucleic acid probe species; each respective nucleic acid probe species in the first plurality of nucleic acid probe species aligns to a different subsequence of the first target region of a reference genome for the species of the subject; the composition comprises, for each respective nucleic acid probe species in the first plurality of nucleic acid probe species, a first amount of a first version of the respective nucleic acid probe species that is conjugated to a capture moiety and a second amount of a second version of the respective nucleic acid probe species that is not conjugated to a capture moiety; the composition comprises a first ratio, for a first respective nucleic acid probe species in the first plurality of the nucleic acid probe species that aligns to a first subsequence of the first target region, of (i) the first amount of the first version of the first respective nucleic acid probe species to (ii) the second amount of the second version of the first respective nucleic acid probe species; the composition comprises a second ratio, for a second respective nucleic acid probe species in the first plurality of the nucleic acid probe species that aligns to a second subsequence of the first target region, of (i) the first amount of the first version of the second respective nucleic acid probe species to (ii) the second amount of the second version of the second respective nucleic acid probe species; and the first ratio is different from the second ratio.
Clause 2. The composition of clause 1, wherein the concentration of the first respective nucleic acid probe species in the first plurality of nucleic acid probe species is equal to the concentration of the second respective nucleic acid probe species in the first plurality of nucleic acid probe species.
Clause 3. The composition of clause 1, wherein the concentration of each respective nucleic acid probe species in the first set of nucleic acid probes is equal in the composition.
Clause 4. The composition of clause 1, wherein the concentration of the first respective nucleic acid probe species in the first plurality of nucleic acid probe sequences is not equal to the concentration of the second respective nucleic acid probe species in the first plurality of nucleic acid probe sequences.
Clause 5. The composition of any one of clauses 1-4, wherein, when the composition is used in a reference nucleic acid pull-down and sequencing assay, the assay outputs an equivalent number of raw sequencing reads of the first subsequence of the first target region and the second subsequence of the first target region.
Clause 6. The composition of any one of clauses 1-4, wherein, when the composition is used in a first reference nucleic acid pull-down and sequencing assay, difference between (i) the number of raw sequencing reads output for the first subsequence of the first target region and (ii) the number of raw sequencing reads output for the second subsequence of the first target region is less than the difference between (iii) the number of raw sequencing reads output for the first subsequence of the first target region in a second reference nucleic acid pull-down and sequencing assay and (iv) the number of raw sequencing reads output for the second subsequence of the first target region in the second reference nucleic acid pull-down and sequencing assay; the first reference nucleic acid pull-down and sequencing assay and the second reference nucleic acid pull-down and sequencing assay are performed using the same methodology; the second reference nucleic acid pull-down and sequencing assay is performed with a second composition comprising the first respective nucleic acid probe species and the second respective probe species; and in the second composition, the percentage of the first respective nucleic acid probe species that are conjugated to the capture moiety and the percentage of the second respective nucleic acid probe species that are conjugated to the capture moiety are the same.
Clause 7. The composition of clause 6, wherein the difference between (i) the number of raw sequencing reads output for the first subsequence of the first target region and (ii) the number of raw sequencing reads output for the second subsequence of the first target region is at least 75% less than the difference between (iii) the number of raw sequencing reads output for the first subsequence of the first target region in the second reference nucleic acid pull-down and sequencing assay and (iv) the number of raw sequencing reads output for the second subsequence of the first target region in the second reference nucleic acid pull-down and sequencing assay.
Clause 8. The composition of any one of clauses 1-7, wherein, when the composition is used in a reference nucleic acid pull-down and sequencing assay, the assay outputs for each respective nucleic acid probe species in the first plurality of nucleic acid probe species a corresponding number of raw sequence reads, thereby forming a first distribution of numbers of raw sequence reads for the respective subsequences of the first target region that align with a respective nucleic acid probe species in the first set of nucleic acid probes; and the range of the first distribution is less than 100% percent of the median of the distribution.
Clause 9. The composition of any one of clauses 1-7, wherein, when the composition is used in a reference nucleic acid pull-down and sequencing assay, the assay outputs for each respective nucleic acid probe species in the first plurality of nucleic acid probe species a corresponding number of raw sequence reads, thereby forming a first distribution of numbers of raw sequence reads for the respective subsequences of the first target region that align with a respective nucleic acid probe species in the first set of nucleic acid probes; and the first distribution has a fold-80 score of less than 1.5.
Clause 10. The composition of any one of clauses 1-7, wherein, when the composition is used in a reference nucleic acid pull-down and sequencing assay, the assay outputs for each respective nucleic acid probe species in the first plurality of nucleic acid probe species a corresponding number of raw sequence reads, thereby forming a first distribution of numbers of raw sequence reads for the respective subsequences of the first target region that align with a respective nucleic acid probe species in the first set of nucleic acid probes; the range of the first distribution is less than the range of a second distribution; the second distribution is determined by using a second composition in the reference nucleic acid pull-down and sequencing assay to output, for each respective nucleic acid probe species in the first plurality of nucleic acid probe species, a corresponding number of raw sequence reads, thereby forming the second distribution of numbers of raw sequence reads for the respective subsequences of the first target region that align with a respective nucleic acid probe species in the first set of nucleic acid probes; and in the second composition, the percentage of each respective nucleic acid probe species in the first plurality of nucleic acid probe species that are conjugated to the capture moiety is the same.
Clause 11. The composition of clause 10, wherein the range of the first distribution is at least 50% less than the range of the second distribution.
Clause 12. The composition of clause 10, wherein the fold-80 score of the first distribution is at least 50% less than the fold-80 score of the second distribution.
Clause 13. The composition of any one of clauses 1-12, wherein the first plurality of nucleic acid probe species is at least 10 nucleic acid probe species.
Clause 14. The composition of any one of clauses 1-13, wherein the first target region comprises a nucleotide, a portion of an intron, a portion of an exon, an intron, an exon, a subset of contiguous exons for a gene, a subset of contiguous exons and introns for a gene, a gene, a portion of a chromosome, an arm of a chromosome, or an entire chromosome.
Clause 15. The method of clause 14, wherein the first target region comprises a gene selected from the group consisting of BRCA1, BRCA2, a CYP gene, CYP2D, a PMS2 pseudogene, a PMSCL pseudogene, DMD, MET, TP53, ALK, IGF1, TLR9, FLT3, and a TCR/BCR gene.
Clause 16. The composition of any one of clauses 1-15, wherein the capture moiety is biotin.
Clause 17. The composition of any one of clauses 1-16, the composition further comprising a second set of nucleic acid probes for identifying a genomic characteristic of a second target region in the genome of the subject, the second set of nucleic acid probes comprises a second plurality of nucleic acid probe species; each respective nucleic acid probe species in the second plurality of nucleic acid probe species aligns to a different subsequence of the second target region of the reference genome for the species of the subject; the composition comprises, for each respective nucleic acid probe species in the second plurality of nucleic acid probe species, a first amount of a first version of the respective nucleic acid probe species that is conjugated to the capture moiety and a second amount of a second version of the respective nucleic acid probe species that is not conjugated to a capture moiety; the composition comprises a third ratio, for a first respective nucleic acid probe species in the second plurality of the nucleic acid probe species that aligns to a first subsequence of the second target region, of (i) the first amount of the first version of the first respective nucleic acid probe species to (ii) the second amount of the second version of the first respective nucleic acid probe species; the composition comprises a fourth ratio, for a second respective nucleic acid probe species in the second plurality of the nucleic acid probe species that aligns to a second subsequence of the second target region, of (i) the first amount of the first version of the second respective nucleic acid probe species to (ii) the second amount of the second version of the second respective nucleic acid probe species; and the third ratio is different from the fourth ratio.
Clause 18. The composition of clause 17, wherein the concentration of the first respective nucleic acid probe species in the second plurality of nucleic acid probe species is equal to the concentration of the second respective nucleic acid probe species in the second plurality of nucleic acid probe species.
Clause 19. The composition of clause 17 or 18, wherein the concentration of the first respective nucleic acid probe species in the second plurality of nucleic acid probe species is equal to the concentration of the first respective nucleic acid probe species in the first plurality of nucleic acid probe species.
Clause 20. The composition of clause 17 or 18, wherein the concentration of the first respective nucleic acid probe species in the second plurality of nucleic acid probe species is not equal to the concentration of the first respective nucleic acid probe species in the first plurality of nucleic acid probe species.
Clause 21. The composition of clause 17, wherein the concentration of the first respective nucleic acid probe species in the second plurality of nucleic acid probe species is not equal to the concentration of the second respective nucleic acid probe species in the second plurality of nucleic acid probe species.
Clause 22. The composition of any one of clauses 17-20, wherein, when the composition is used in a reference nucleic acid pull-down and sequencing assay, the assay outputs an equivalent number of raw sequencing reads of the first subsequence of the second target region and the second subsequence of the second target region.
Clause 23. The composition of any one of clauses 17-22, wherein the first ratio is different from the third ratio and the fourth ratio.
Clause 24. The composition of any one of clauses 17-23, wherein the second ratio is different from the third ratio and the fourth ratio.
Clause 25. The composition of any one of clauses 17-24, wherein, when the composition is used in a reference nucleic acid pull-down and sequencing assay, the assay outputs an equivalent number of raw sequencing reads of the first subsequence of the first target region and the first subsequence of the second target region.
Clause 26. The composition of clause 17, wherein the concentration of each respective nucleic acid probe species in the second set of nucleic acid probes is equal in the composition.
Clause 27. The composition of any one of clauses 17-26, wherein, when the composition is used in a reference nucleic acid pull-down and sequencing assay, the assay outputs for each respective nucleic acid probe species in the second plurality of nucleic acid probe species a corresponding number of raw sequence reads, thereby forming a second distribution of numbers of raw sequence reads for the respective subsequences of the second target region that align with a respective nucleic acid probe species in the second set of nucleic acid probes; and the range of the second distribution is less than 100% of the median of the distribution.
Clause 28. The composition of any one of clauses 17-26, wherein, when the composition is used in a reference nucleic acid pull-down and sequencing assay, the assay outputs for each respective nucleic acid probe species in the second plurality of nucleic acid probe species a corresponding number of raw sequence reads, thereby forming a second distribution of numbers of raw sequence reads for the respective subsequences of the second target region that align with a respective nucleic acid probe species in the second set of nucleic acid probes; and the second distribution has a fold-80 score of less than 1.5.
Clause 29. The composition of any one of clauses 17-28, wherein the first plurality of nucleic acid probe species is at least 10 nucleic acid probe species.
Clause 30. The composition of any one of clauses 17-29, wherein the first target region comprises a human gene selected from the group consisting of BRCA1, BRCA2, a CYP gene, CYP2D, a PMS2 pseudogene, a PMSCL pseudogene, DMD, MET, TP53, ALK, IGF1, TLR9, FLT3, and a TCR/BCR gene.
Clause 31. A method for determining a genomic characteristic of a subject, the method comprising contacting a sample comprising nucleic acids from the subject with a composition according to any one of clauses 1-28; recovering a portion of the nucleic acids using an agent that binds to the capture moiety; and sequencing the recovered portion of the nucleic acids, thereby identifying a genomic characteristic of the subject.
Clause 32. The method of clause 31, wherein the genomic characteristic is selected from the group consisting of a single nucleotide variant (SNV), an indel, a copy number variation (CNV), a pseudogene, a CG-rich region, an AT-rich region, a genetic rearrangement, a splice variant, a gene expression level, aneuploidy, and trisomy.
Clause 33. The method of clause 31 or 32, wherein the nucleic acids from the subject are obtained from a liquid biological sample from the subject.
Clause 34. The method of clause 33, wherein the liquid biological sample is a blood sample or a blood plasma sample from the subject.
Clause 35. The method of clause 31 or 32, wherein the nucleic acids from the subject are obtained from a solid biological sample from the subject.
Clause 36. The method of clause 35, wherein the solid biological sample is a tumor sample or a normal tissue sample from the subject.
Clause 37. The method of any one of clauses 31-36, wherein the nucleic acids comprise mRNA or cDNA generated from mRNA, the method further comprising, prior to contacting the sample with the composition, selectively removing a portion of the mRNA or cDNA from a first gene that is represented in the sample at a level that is greater than the representation of at least 50% of the genes represented in the sample.
Clause 38. The method of clause 37, wherein the first gene is represented in the sample at a level that is greater than the representation of at least 75% of the genes represented in the sample.
Clause 39. A method for determining a genomic characteristic of a subject, the method comprising identifying a first genomic characteristic of the subject from a first sample comprising nucleic acids from the subject by contacting the first sample comprising nucleic acids from the subject with a first composition according to any one of clauses 1-28, recovering a portion of the nucleic acids from the first sample using an agent that binds to the capture moiety, and sequencing the portion of the nucleic acids recovered from the first sample; and identifying a second genomic characteristic of the subject from a second sample comprising nucleic acids from the subject by contacting the second sample comprising nucleic acids from the subject with a second composition according to any one of clauses 1-28, recovering a portion of the nucleic acids from the second sample using an agent that binds to the capture moiety, and sequencing the portion of the nucleic acids recovered from the second sample; wherein the first set of nucleic acid probes in the first composition and the first set of nucleic acid probes in the second composition align to the same target region of the reference genome for the species of the subject, the first respective nucleic acid probe species in the first plurality of the nucleic acid probe species in the first composition and the first respective nucleic acid probe species in the first plurality of the nucleic acid probe species in the second composition align to the same subsequence of the same target region, and the first ratio for the first respective nucleic acid probe species in the first plurality of the nucleic acid probe species in the first composition is different than the first ratio for the first respective nucleic acid probe species in the first plurality of the nucleic acid probe species in the second composition.
Clause 40. The method of clause 39, wherein the nucleic acids in the first sample are obtained from a biological sample from a first tissue in the subject and the nucleic acids in the second sample are obtained from a biological sample obtained from a second tissue in the subject.
Clause 41. The method of clause 39 or 40, wherein the nucleic acids in the first sample are obtained from a solid biological sample from the subject and the nucleic acids in the second sample are obtained from a liquid biological sample from the subject.
Clause 42. The method of clause 41, wherein the solid biological sample is a tumor sample or a normal tissue sample from the subject.
Clause 43. The method of clause 40 or 42, wherein the liquid biological sample is a blood sample or a blood plasma sample from the subject.
Clause 44. The method of clause 39 or 40, wherein the nucleic acids in the first sample are DNA and the nucleic acids in the second sample are RNA.
Clause 45. The method of clause 39 or 40, wherein the nucleic acids in the first sample represent a whole exome from the subject and the nucleic acids in the second sample represent a targeted panel of nucleic acid sequences from the subject.
Clause 46. A method for designing a uniform probe set, comprising (A) obtaining an initial set of probes, where each probe in the set of probes corresponds to a region of a reference genome, and each probe has a respective concentration; (B) analyzing the initial set of probes against a sample library, thereby obtaining at least i) a respective recovery rate for each probe in the initial set of probes, ii) a median recovery rate for the initial set of probes, and iii) a subset of probes, where the respective recovery rate of each probe in the subset of probes does not satisfy a predetermined recovery rate threshold; (C) modifying, for each probe in the subset of probes, the respective concentration of said probe, thereby obtaining an updated set of probes; (D) analyzing the updated set of probes against the sample library, thereby obtaining at least i) a respective updated recovery rate for each probe in the updated set of probes, ii) a median recovery rate for the updated set of probes, and iii) a subset of probes, where the respective recovery rate of each probe in the subset of probes does not satisfy a predetermined recovery rate threshold; and (E) repeating the modifying (C) and analyzing (D) until the respective updated recovery rate for each probe in the updated set of probes satisfies the predetermined recovery rate threshold, thereby providing the optimized set of probes for the sample library.

### Systems and Methods of Balancing Probe Sets.

### Example System Embodiments

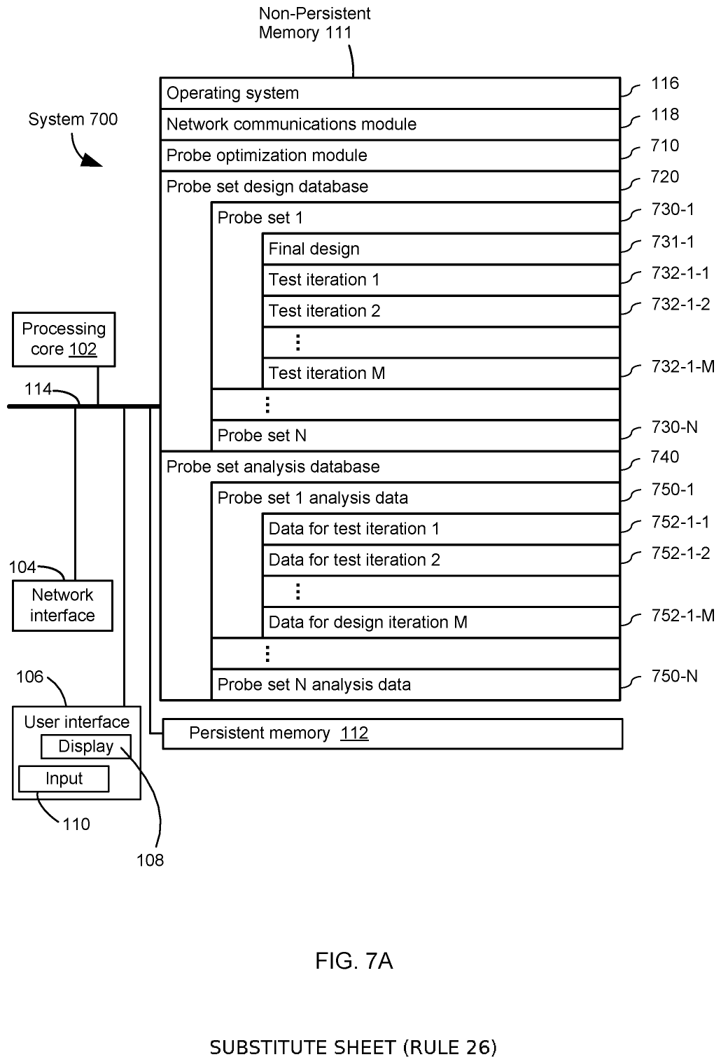

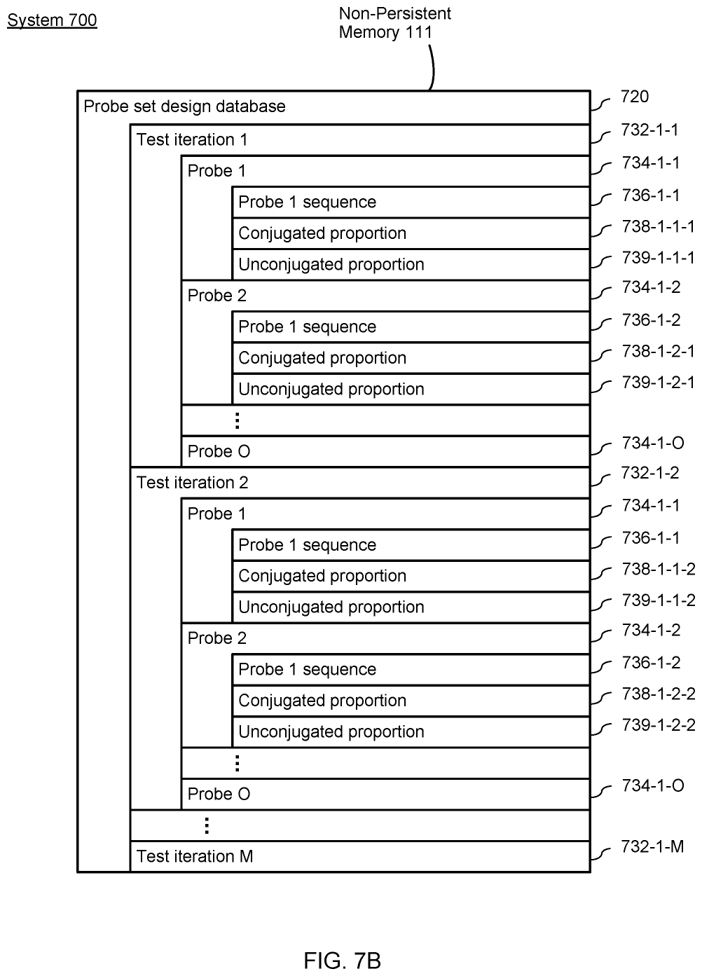

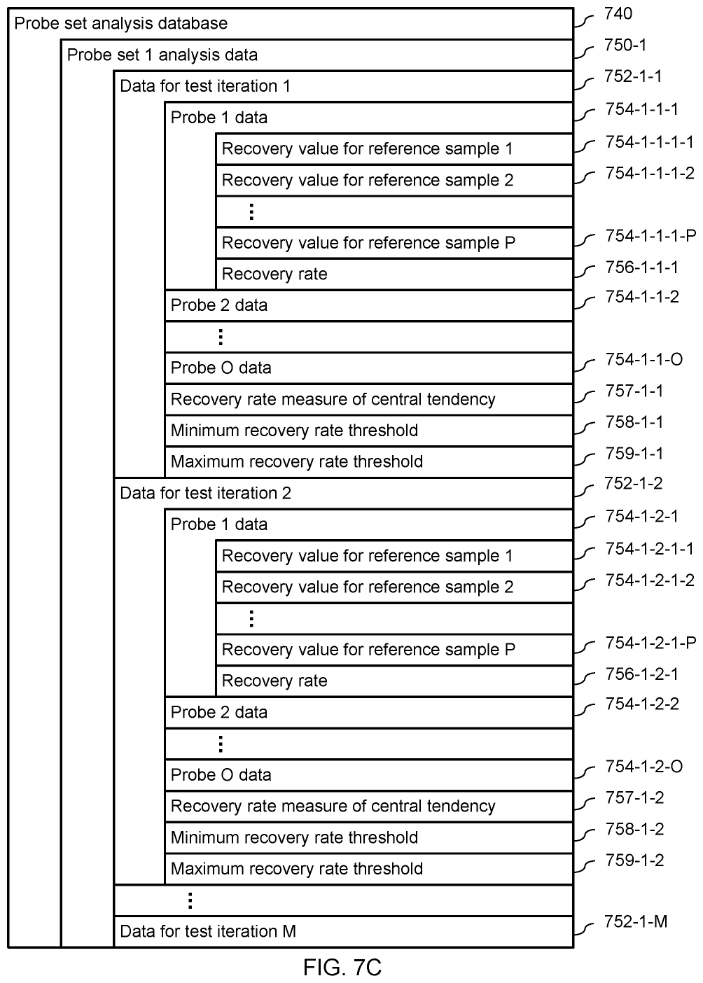

Now that an overview of some aspects of the present disclosure and some definitions used in the present disclosure have been provided, details of an exemplary system are described in conjunction with FIGS. 7A, 7B, and 7C. FIGS. 7A-7C collectively illustrate a block diagram illustrating a system 700 in accordance with some implementations. The system 700 in some implementations includes one or more processing units CPU(s) 102 (also referred to as processors), one or more network interfaces 104, a user interface 106 including (optionally) a display 108 and an input system 110, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 700 with other devices and/or a communication network 104;
a probe optimization module 710 for determining an optimized set of probes for use against a sample (e.g., a nucleic acid sample from a patient);
a probe set design database 720 storing a first nucleic acid probe set 730 for enriching a plurality of genomic loci, where the first nucleic acid probe set has a final design 731 (e.g., 731-1) and a plurality of test iterations 732 (e.g., 732-1-1, 732-1-2, . . . 732-1-M), and, for each respective probe 734 in a plurality of probes for the first nucleic acid probe set (e.g., 734-1-1, 734-1-2, . . . 734-1-0), each respective test iteration in the plurality of test iterations comprises a respective probe sequence 736 (e.g., 736-1-1, 736-1-2) that aligns to a different subsequence of a respective locus in the plurality of genomic loci, a respective first proportion of a conjugated version 738 of the respective probe (e.g., 738-1-1-1, 738-1-2-1, 738-1-1-2, 738-1-2-2), and a respective second proportion of an unconjugated version 739 of the respective probe (e.g., 739-1-1-1, 739-1-2-1, 739-1-1-2, 739-1-2-2); and
a probe set analysis database 740 storing analysis data for the first probe set 750, where the analysis data for the first probe set includes, for each respective test iteration 752 in the plurality of test iterations (e.g., 752-1-1, 752-1-2, . . . 752-1-M), a recovery rate measure of central tendency 757 (e.g., 757-1-1, 757-1-2), a minimum recovery rate threshold 758 (e.g., 758-1-1, 758-1-2), a maximum recovery rate threshold 759 (e.g., 759-1-1, 759-1-2), and, for each respective probe in the plurality of probes, a corresponding recovery rate 756 (e.g., 756-1-1-1, 756-1-2-1) and a plurality of recovery values 754 obtained against a first plurality of reference nucleic acid samples (e.g., 754-1-1-1, . . . 754-1-2-O).

Optionally, the probe set design database 720 comprises a plurality of probe sets 730 (e.g., 730-1, . . . 730-N), each respective probe set in the plurality of probe sets including a respective final design for the probe set 731 and a respective plurality of test iterations 732. Optionally, the probe set analysis database 740 comprises a respective set of analysis data 750 for each probe set in the plurality of probe sets (e.g., 750-1, . . . 750-N), each respective set of analysis data including data for each respective test iteration in the plurality of test iterations 752.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 700, that is addressable by visualization system 700 so that visualization system 700 may retrieve all or a portion of such data when needed.

Although FIGS. 7A-7C depict a “system 700,” the figure is intended more as a functional description of the various features that may be present in computer systems than as a structural schematic of the implementations described herein. In practice, and as recognized by those of ordinary skill in the art, items shown separately could be combined and some items could be separated. Moreover, although FIGS. 7A-7C depict certain data and modules in non-persistent memory 111, some or all of these data and modules instead may be stored in persistent memory 112.

### Balancing Probe Sets

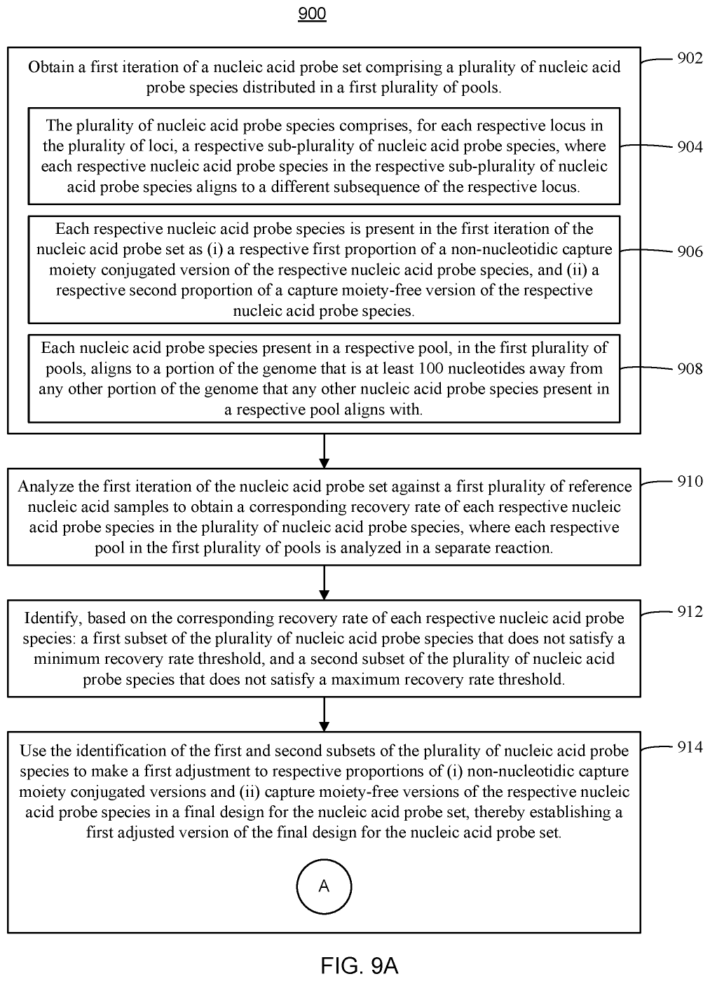

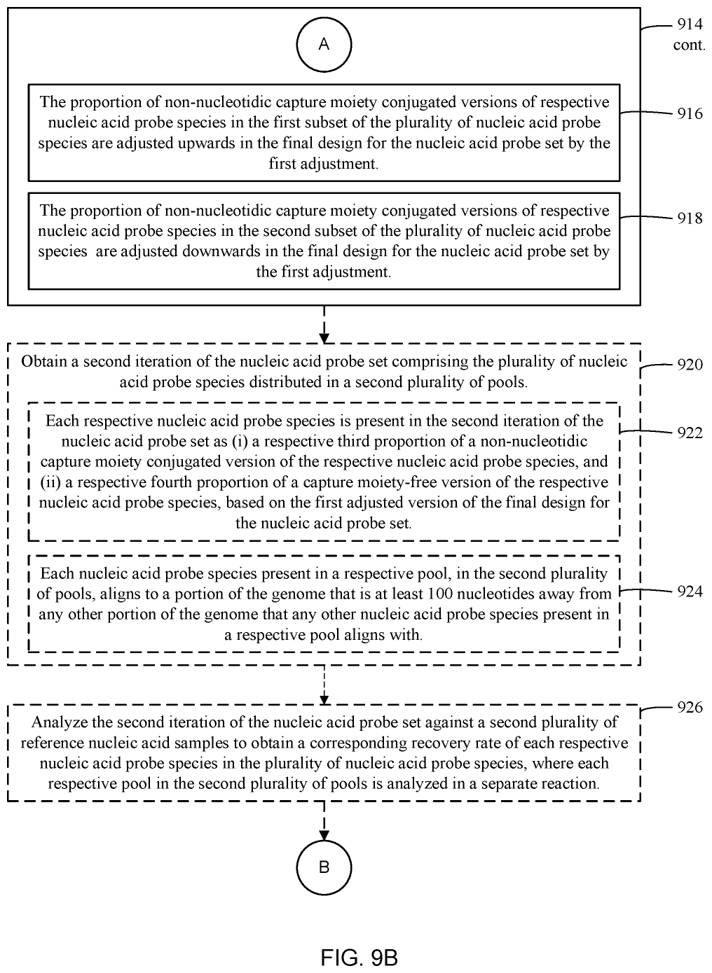

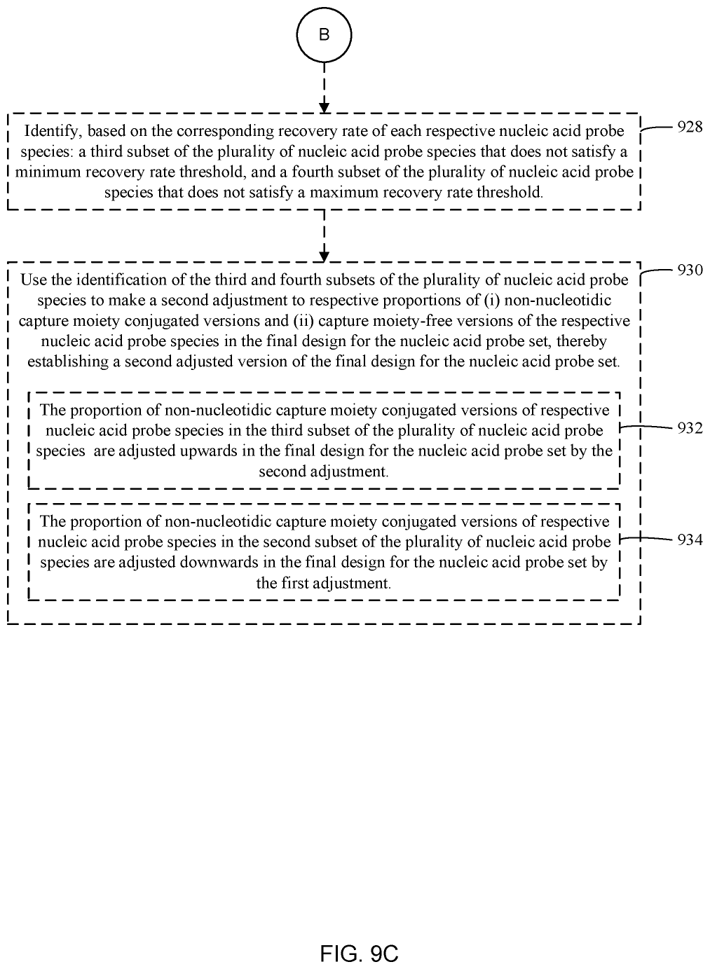

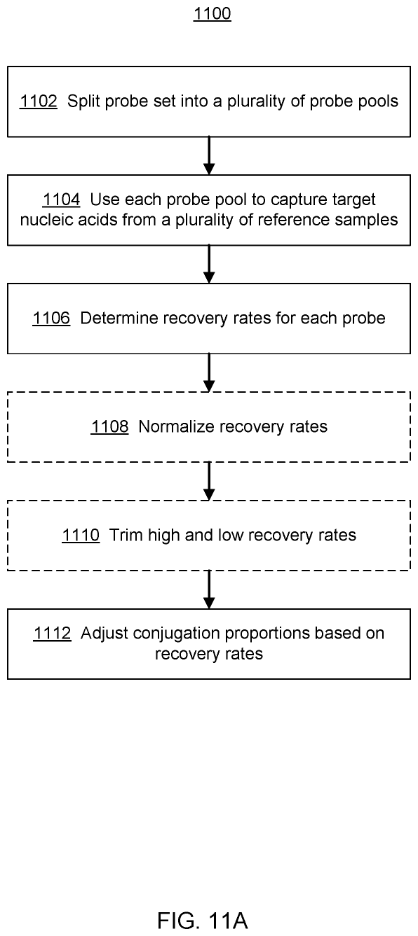

While a system in accordance with the present disclosure has been disclosed with reference to FIGS. 7A-7C, methods in accordance with the present disclosure are now detailed below with reference to FIGS. 9A-9C and FIG. 11. FIGS. 9A-9C collectively provide an example outline of a method 900 for balancing a probe set for enriching a plurality of genomic loci, in accordance with some embodiments described herein. Similarly, FIG. 11 provides an example outline of a method 1100 for balancing a probe set for enriching a plurality of genomic loci, in accordance with some embodiments described herein.

Referring to Block 902, the method comprises obtaining a first iteration of a nucleic acid probe set comprising a plurality of nucleic acid probe species distributed in a first plurality of pools.

In some embodiments, the plurality of genomic loci comprises at least 100 loci. In some embodiments, the plurality of genomic loci is at least 10, at least 15, at least 25, at least 30, at least 40, at least 50, at least 100, at least 200, at least 250, at least 400, at least 500, at least 600, at least 700, at least 800, at least 900, at least 1000, at least 2000, at least 2500, at least 4000, at least 5000, at least 6000, at least 7000, at least 8000, at least 9000, at least 10,000, at least 15,000, or at least 20,000 loci. In some embodiments, the plurality of genomic loci is no more than 30,000, no more than 20,000, no more than 10,000, no more than 8000, no more than 7500, no more than 5000, no more than 4000, no more than 3000, no more than 2000, no more than 1000, no more than 750, no more than 500, no more than 250, no more than 100, no more than 50, or no more than 25 loci. In some embodiments, the plurality of genomic loci is from 10 to 50, from 25 to 100, from 100 to 500, from 100 to 1000, from 1000 to 2000, from 10 to 500, from 500 to 2000, from 1000 to 5000, from 5000 to 10,000, or from 10,000 to 20,000 loci. In some embodiments, the plurality of genomic loci is from 10 to 100,000 loci, from 100 to 100,000 loci, from 1000 to 100,000 loci, from 5000 to 100,000 loci, from 10,000 to 100,000 loci, or from 50,000 to 100,000 loci. In some embodiments, the plurality of genomic loci is from 10 to 50,000 loci, from 100 to 50,000 loci, from 1000 to 50,000 loci, from 5000 to 50,000 loci, or from 10,000 to 50,000 loci. In some embodiments, the plurality of genomic loci is from 10 to 30,000 loci, from 100 to 30,000 loci, from 1000 to 30,000 loci, from 5000 to 30,000 loci, or from 10,000 to 30,000 loci. In some embodiments, the plurality of genomic loci is from 10 to 10,000 loci, from 100 to 10,000 loci, from 1000 to 10,000 loci, or from 5000 to 10,000 loci. In some embodiments, the plurality of genomic loci is from 10 to 1000 loci, from 100 to 1000 loci, or from 500 to 1000 loci. In some embodiments, the plurality of genomic loci falls within another range starting no lower than 10 loci and ending no higher than 30,000 loci.

In some embodiments, a genomic locus in the plurality of genomic loci is a gene. In some embodiments, each genomic locus in the plurality of genomic loci is a gene. In some embodiments, the plurality of loci includes a whole exome. In some embodiments, the plurality of loci includes a whole human exome. In some embodiments, the plurality of loci includes all, or substantially all (e.g., at least 98%, at least 99%, at least 99.5%, or at least 99.9%), of a chromosomal arm. For example, in some embodiments, an entire chromosomal arm is covered by a probe set except for one or more complex genomic regions, such as a telomere, telomeric region, kinetochore, kinetochoric region, large nucleotide repeat, and the like. In some embodiments, the plurality of loci includes all, or substantially all (e.g., at least 98%, at least 99%, at least 99.5%, or at least 99.9%), of a chromosome. For example, in some embodiments, an entire chromosome is covered by a probe set except for one or more complex genomic regions, such as a telomere, telomeric region, kinetochore, kinetochoric region, large nucleotide repeat, and the like. In some embodiments, the plurality of loci includes all, or substantially all (e.g., at least 98%, at least 99%, at least 99.5%, or at least 99.9%), of a plurality of chromosomes, e.g., 2, 3, 4, 5, 6, 7, 8, 9, 10, 11, 12, 13, or more chromosomes. In some embodiments, the plurality of loci includes all, or substantially all, of a genome.

In some embodiments, the plurality of nucleic acid probe species is at least 2000 nucleic acid probe species. In some embodiments, the plurality of nucleic acid probe species is at least 100, at least 200, at least 250, at least 400, at least 500, at least 600, at least 700, at least 800, at least 900, at least 1000, at least 2000, at least 2500, at least 4000, at least 5000, at least 6000, at least 7000, at least 8000, at least 9000, at least 10,000, at least 15,000, at least 20,000, at least 50,000, at least 70,000, at least 100,000, at least 200,000, at least 300,000, at least 400,000, at least 500,000, at least 600,000, at least 700,000, at least 800,000, at least 900,000, at least 1,000,000, at least 2,500,000, or at least 5,000,000 nucleic acid probe species. In some embodiments, the plurality of nucleic acid probe species is no more than 5,000,000, no more than 2,500,000, no more than 1,000,000, no more than 900,000, no more than 750,000, no more than 500,000, no more than 250,000, no more than 100,000, no more than 75,000, no more than 50,000, no more than 25,000, no more than 20,000, no more than 10,000, no more than 8000, no more than 7500, no more than 5000, no more than 4000, no more than 3000, no more than 2000, no more than 1000, no more than 750, no more than 500, no more than 250, no more than 100, no more than 50, or no more than 25 nucleic acid probe species. In some embodiments, the plurality of nucleic acid probe species is from 100 to 500, from 250 to 1000, from 1000 to 5000, from 1000 to 10,000, from 10,000 to 20,000, from 10,000 to 50,000, from 50,000 to 200,000, from 100,000 to 500,000, from 500,000 to 1,000,000, from 100,000 to 1,000,000, or from 1,000,000 to 5,000,000 nucleic acid probe species. In some embodiments, the plurality of nucleic acid probe species is from 100 to 10,000,000, from 1000 to 10,000,000, from 10,000 to 10,000,000, from 100,000 to 10,000,000, or from 1,000,000 to 10,000,000 nucleic acid probe species. In some embodiments, the plurality of nucleic acid probe species is from 100 to 5,000,000, from 1000 to 5,000,000, from 10,000 to 5,000,000, from 100,000 to 5,000,000, or from 1,000,000 to 5,000,000 nucleic acid probe species. In some embodiments, the plurality of nucleic acid probe species is from 100 to 1,000,000, from 1000 to 1,000,000, from 10,000 to 1,000,000, or from 100,000 to 1,000,000 nucleic acid probe species. In some embodiments, the plurality of nucleic acid probe species is from 100 to 500,000, from 1000 to 500,000, from 10,000 to 500,000, or from 100,000 to 500,000 nucleic acid probe species. In some embodiments, the plurality of nucleic acid probe species is from 100 to 100,000, from 1000 to 100,000, or from 10,000 to 100,000 nucleic acid probe species. In some embodiments, the plurality of nucleic acid probe species is from 100 to 10,000, from 1000 to 10,000, or from 5,000 to 10,000 nucleic acid probe species. In some embodiments, the plurality of nucleic acid probe species is from 100 to 1000 or from 500 to 1000 nucleic acid probe species. In some embodiments, the plurality of nucleic acid probe species falls within another range starting no lower than 100 nucleic acid probe species and ending no higher than 10,000,000 nucleic acid probe species.

In some embodiments, the concentration (e.g., molarity) of each probe in a probe set (e.g., in a test iteration of the probe set or in a final design for the probe set) is the same. In some embodiments, the concentration of at least 85% of the probe species in a probe set (e.g., in a test iteration of the probe set or in a final design for the probe set) are the same. In some embodiments, the concentration of at least 90% of the probe species in a probe set (e.g., in a test iteration of the probe set or in a final design for the probe set) are the same. In some embodiments, the concentration of at least 95% of the probe species in a probe set (e.g., in a test iteration of the probe set or in a final design for the probe set) are the same. In some embodiments, the concentration of at least 99% of the probe species in a probe set (e.g., in a test iteration of the probe set or in a final design for the probe set) are the same.

Accordingly, in some embodiments, each nucleic acid probe species in the plurality of nucleic acid probe species is present in the same amount in the first iteration of the probe set. In some embodiments, the copy number of each nucleic acid probe species in the plurality of nucleic acid probe species is the same in the first iteration of the probe set. In some embodiments, the molarity of each nucleic acid probe species in the plurality of nucleic acid probe species is the same in the first iteration of the probe set. In some embodiments, two or more nucleic acid probe species in the plurality of nucleic acid probe species are present in a different amount in the first iteration of the probe set.

In some embodiments, the plurality of pools comprises at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, or at least 100 pools. In some embodiments, the plurality of pools comprises no more than 150, no more than 90, no more than 80, no more than 70, no more than 60, no more than 50, no more than 40, no more than 30, no more than 20, or no more than 10 pools. In some embodiments, the plurality of pools comprises from 3 to 5, from 3 to 10, from 10 to 50, from 10 to 100, from 10 to 20, from 15 to 75, from 5 to 20, from 20 to 90, or from 3 to 100 pools. In some embodiments, the plurality of pools falls within another range starting no lower than 3 pools and ending no higher than 150 pools.

In some embodiments, for the first iteration of the nucleic acid probe set, each nucleic acid probe species in the plurality of nucleic acid probe species is present in only one pool in the plurality of pools. In some embodiments, for the first iteration of the nucleic acid probe set, each nucleic acid probe species in the plurality of nucleic acid probe species is present in more than one pool in the plurality of pools.

In some embodiments, the pools are formed by including every other probe species into a different pool, based on the location to which the probe species aligns in the corresponding locus. In some embodiments, every third probe, every fourth probe, every fifth probe, etc., into a different pool. In this fashion, a sequence gap is created between the nearest probe species in the pool. In some embodiments, the pooling is done such that no, or substantially no, nucleic acid fragments in the nucleic acid sample being enriched can anneal to more than one probe species in a given pool.

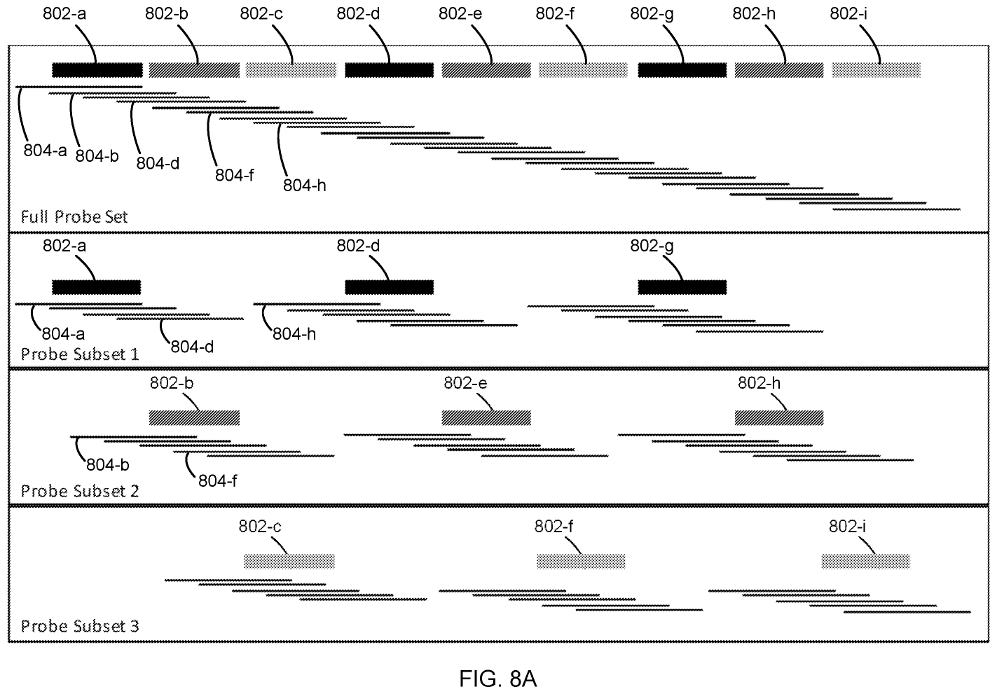

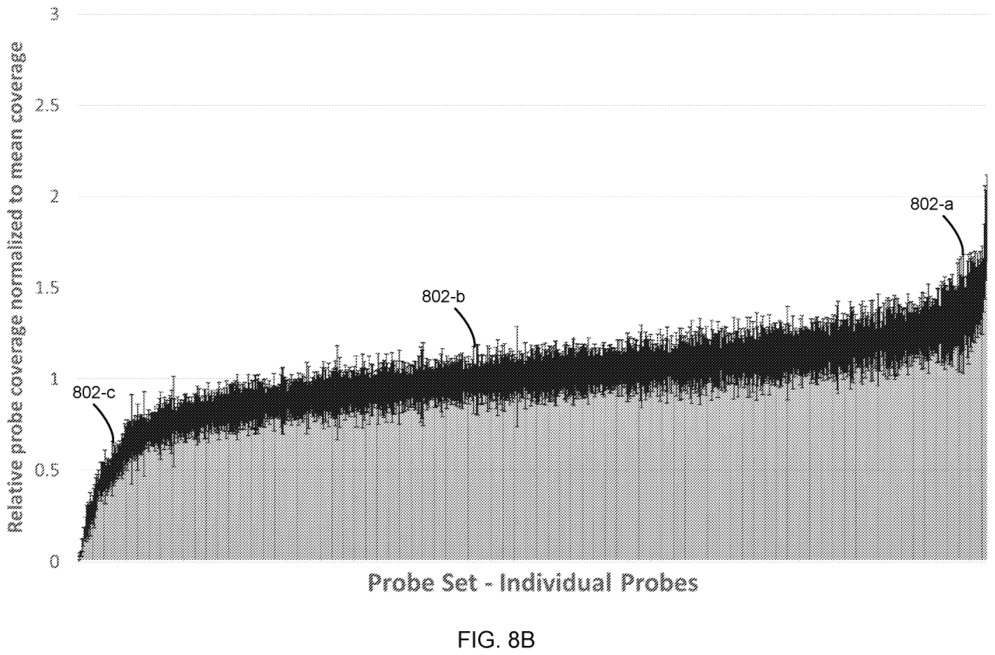

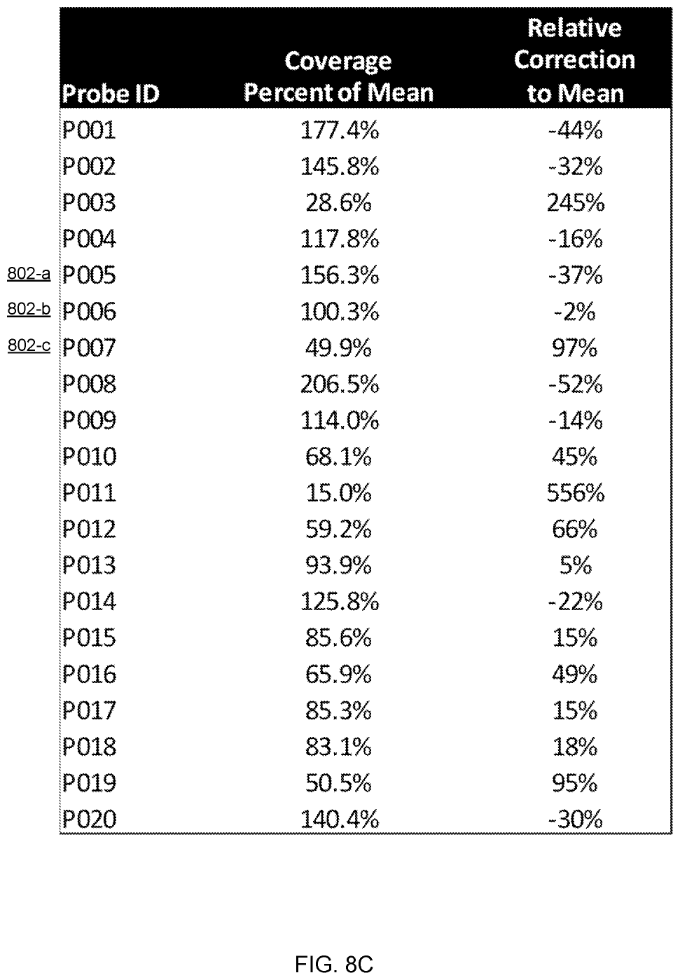

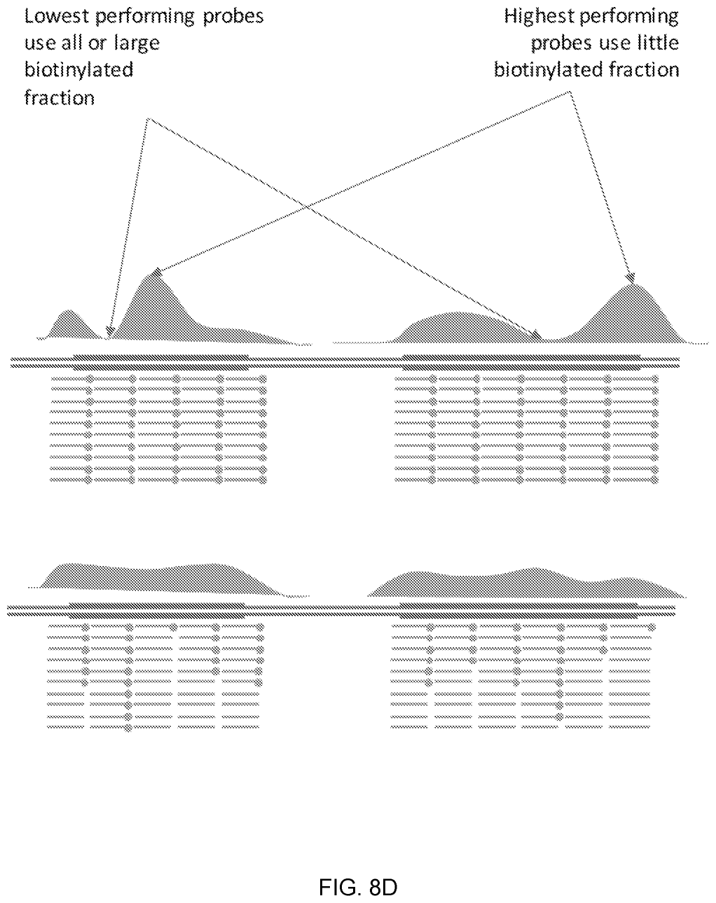

Thus, in some embodiments, the nucleotide gap left between probe species in a pool is selected based on the average or distribution of the size of the nucleic acid fragments in the nucleic acid sample. Taking as an example, a probe set having adjacent, but non-overlapping probes having a uniform length of 120 nucleotides for enrichment of nucleic acid fragments having a distribution of from 100-150 nucleotides, pools should be generate in which at least two probes are left out between the nearest adjacent probes in the pool. For example, FIG. 8A illustrates an embodiment where every third probe species is pooled into one of three pools. As shown on the top panel of FIG. 8A, probes 802-a to 802-i align along a locus represented by a plurality of nucleic acid fragments 804. A first pool (probe subset 1) contains the first of every three probe species, i.e., probe species 802-a, 802-d, and 802-g. A second pool (probe subset 2) contains the second of every three probe species, i.e., probe species 802-b, 802-e, and 802-h. A third pool (probe subset 3) contains the third of every three probe species, i.e., probe species 802-c, 802-f, and 802-i. Assuming the scenario described above, adjacent probe species in each pool (e.g., probes 802-a and 802-d) would be separated by 240 nucleotides. Thus, because nucleotide fragments 804 are no more than 150 nucleotides in length, no fragment 804 can be captured by two different probe species in the pool.

In some embodiments, each pool in the plurality of pools comprises at least 3 nucleic acid probe species. In some embodiments, each pool in the plurality of pools comprises between 3 and 1,000,000 nucleic acid probe species. In some embodiments, each pool in the plurality of pools comprises between 100 and 100,000 nucleic acid probe species.

In some embodiments, each pool in the plurality of pools comprises at least 3, at least 10, at least 15, at least 25, at least 30, at least 40, at least 50, at least 100, at least 200, at least 250, at least 400, at least 500, at least 600, at least 700, at least 800, at least 900, at least 1000, at least 2000, at least 2500, at least 4000, at least 5000, at least 6000, at least 7000, at least 8000, at least 9000, at least 10,000, at least 15,000, at least 20,000, at least 50,000, at least 70,000, at least 100,000, at least 200,000, at least 300,000, at least 400,000, at least 500,000, at least 600,000, at least 700,000, at least 800,000, at least 900,000, or at least 1,000,000 nucleic acid probe species. In some embodiments, each pool in the plurality of pools comprises no more than 900,000, no more than 750,000, no more than 500,000, no more than 250,000, no more than 100,000, no more than 75,000, no more than 50,000, no more than 25,000, no more than 20,000, no more than 10,000, no more than 7500, no more than 5000, no more than 4000, no more than 3000, no more than 2000, no more than 1000, no more than 750, no more than 500, no more than 250, no more than 100, no more than 50, or no more than 20 nucleic acid probe species. In some embodiments, each pool in the plurality of pools comprises from 3 to 500, from 250 to 1000, from 1000 to 5000, from 1000 to 10,000, from 10,000 to 20,000, from 10,000 to 50,000, from 50,000 to 200,000, from 100,000 to 500,000, from 500,000 to 1,000,000, or from 100,000 to 1,000,000 nucleic acid probe species. In some embodiments, each pool in the plurality of pools comprises a plurality of nucleic acid probe species that falls within another range starting no lower than 3 nucleic acid probe species and ending no higher than 1,000,000 nucleic acid probe species.

In some embodiments, the distribution of the plurality of nucleic acid probe species into the first plurality of pools is determined (e.g., distributed) based on the genomic distance between each nucleic acid probe species in the plurality of nucleic acid probe species, aligned to a reference genome. For example, in some embodiments, the distribution of the plurality of nucleic acid probe species into the first plurality of pools is determined by placing no two nucleic acid probe species into a single respective pool if the genomic distance between the two nucleic acid probe species is less than a threshold distance.

In some embodiments, the distribution of the plurality of nucleic acid probe species into the first plurality of pools is determined based on a predetermined number of pools. In some such embodiments, the distribution of the plurality of nucleic acid probe species into the first plurality of pools is determined by placing each subsequent nucleic acid probe species, in order of their alignment to a reference genome, into each subsequent pool in the predetermined number of pools, in order, until each pool has been added to. Adding the remaining nucleic acid probe species is then repeated for the predetermined number of pools, in order, starting with the first pool.

For instance, as illustrated in FIG. 8A and discussed in greater detail in Example 2 below, in some embodiments, the plurality of pools is three pools, and each of the respective three pools contains, for a respective genomic locus in the plurality of genomic loci, every third respective nucleic acid probe species in the respective sub-plurality of nucleic acid probe species that align to the respective locus, such that the three pools collectively contain every respective nucleic acid probe species in the respective sub-plurality of nucleic acid probe species that align to the respective locus.

Referring to Block 904, the plurality of nucleic acid probe species comprises, for each respective locus in the plurality of loci, a respective sub-plurality of nucleic acid probe species, where each respective nucleic acid probe species in the respective sub-plurality of nucleic acid probe species aligns to a different subsequence of the respective locus.

In some embodiments, each respective nucleic acid probe species in the plurality of nucleic acid probe species comprises a respective nucleic acid sequence of from 75 nucleotides to 250 nucleotides that aligns with the respective subsequence of the respective locus. In some embodiments, each respective nucleic acid probe species in the plurality of nucleic acid probe species comprises a respective nucleic acid sequence of from 25 nucleotides to 500 nucleotides that aligns with the respective subsequence of the respective locus. In some embodiments, each respective nucleic acid probe species in the plurality of nucleic acid probe species comprises a respective nucleic acid sequence of from 50 nucleotides to 500 nucleotides, of from 75 nucleotides to 500 nucleotides, of from 100 nucleotides to 500 nucleotides, of from 125 nucleotides to 500 nucleotides, of from 150 nucleotides to 500 nucleotides, of from 200 nucleotides to 500 nucleotides, or of from 250 nucleotides to 500 nucleotides that aligns with the respective subsequence of the respective locus. In some embodiments, each respective nucleic acid probe species in the plurality of nucleic acid probe species comprises a respective nucleic acid sequence of from 25 nucleotides to 250 nucleotides, of from 50 nucleotides to 250 nucleotides, of from 75 nucleotides to 250 nucleotides, of from 100 nucleotides to 250 nucleotides, of from 125 nucleotides to 250 nucleotides, of from 150 nucleotides to 250 nucleotides, or of from 200 nucleotides to 250 nucleotides that aligns with the respective subsequence of the respective locus. In some embodiments, each respective nucleic acid probe species in the plurality of nucleic acid probe species comprises a respective nucleic acid sequence of from 25 nucleotides to 200 nucleotides, of from 50 nucleotides to 200 nucleotides, of from 75 nucleotides to 200 nucleotides, of from 100 nucleotides to 200 nucleotides, of from 125 nucleotides to 200 nucleotides, or of from 150 nucleotides to 200 nucleotides that aligns with the respective subsequence of the respective locus. In some embodiments, each respective nucleic acid probe species in the plurality of nucleic acid probe species comprises a respective nucleic acid sequence of from 25 nucleotides to 150 nucleotides, of from 50 nucleotides to 150 nucleotides, of from 75 nucleotides to 150 nucleotides, of from 100 nucleotides to 150 nucleotides, or of from 125 nucleotides to 150 nucleotides that aligns with the respective subsequence of the respective locus. In some embodiments, each respective nucleic acid probe species in the plurality of nucleic acid probe species comprises a respective nucleic acid sequence of from 25 nucleotides to 125 nucleotides, of from 50 nucleotides to 125 nucleotides, of from 75 nucleotides to 125 nucleotides, or of from 100 nucleotides to 125 nucleotides that aligns with the respective subsequence of the respective locus.

In some embodiments, the sub-plurality of nucleic acid probe species for a respective locus in the plurality of loci consists of non-overlapping nucleic acid probe sequences. In some embodiments, the gap between any two respective nucleic acid probe species in a sub-plurality of probe species (e.g., those probe species that align to a particular locus) that align to adjacent subsequences in a respective locus is no more than 50, no more than 40, no more than 30, no more than 20, no more than 10, or no more than 5 nucleotides.

In some embodiments, the sub-plurality of nucleic acid probe species for a respective locus in the plurality of loci consists of overlapping nucleic acid probe sequences. In some embodiments, the sub-plurality of nucleic acid probe species for a respective locus in the plurality of loci covers the respective locus at a coverage of at least 0.75×, at least 0.9×, at least 0.95×, at least 1×, at least 1.5×, at least 2×, at least 2.5×, at least 3×, at least 3.5×, at least 4×, at least 4.5×, at least 5×, at least 6×, at least 7×, at least 8×, at least 9×, at least 10×, at least 15×, at least 20×, at least 25×, or at least 30×.

Referring to Block 906, each respective nucleic acid probe species is present in the first iteration of the nucleic acid probe set as (i) a respective first proportion of a non-nucleotidic capture moiety conjugated version of the respective nucleic acid probe species, and (ii) a respective second proportion of a capture moiety-free version of the respective nucleic acid probe species.

In some embodiments, a non-nucleotidic capture moiety is covalently attached to a nucleic acid probe in the plurality of nucleic acid probe species. In some embodiments, a non-nucleotidic capture moiety is an affinity moiety used for recovering and/or detecting a respective nucleic acid probe species. In some embodiments, non-limiting examples of non-nucleotidic capture moieties include biotin, digoxigenin, and dinitrophenol. In some embodiments, the capture moiety is biotin.

In some embodiments, in the first iteration of the probe set, the first proportion of the non-nucleotidic capture moiety-conjugated version of each nucleic acid probe species in the plurality of nucleic acid probe species is the same. For instance, in some embodiments, in the first iteration of the probe set, the first proportion of the non-nucleotidic capture moiety-conjugated version of each nucleic acid probe species in the plurality of nucleic acid probe species is 50%.

In some embodiments, in the first iteration of the probe set, the first proportion of the non-nucleotidic capture moiety-conjugated version of a nucleic acid probe species in the plurality of nucleic acid probe species is at least 5%, at least 10%, at least 15%, at least 20%, at least 30%, at least 40%, at least 50%, at least 60%, at least 70%, at least 80%, at least 90%, or at least 95%. In some embodiments, the first proportion of the non-nucleotidic capture moiety-conjugated version of a nucleic acid probe species in the plurality of nucleic acid probe species is no more than 99%, no more than 95%, no more than 90%, no more than 80%, no more than 70%, no more than 60%, no more than 50%, no more than 40%, no more than 30%, or no more than 20%. In some embodiments, the first proportion of the non-nucleotidic capture moiety-conjugated version of a nucleic acid probe species in the plurality of nucleic acid probe species is from 5% to 95%, from 10% to 90%, from 20% to 80%, from 30% to 70%, from 40% to 60%, or from 45% to 55%. In some embodiments, the first proportion of the non-nucleotidic capture moiety-conjugated version of a nucleic acid probe species in the plurality of nucleic acid probe species is 100%. In some embodiments, the first proportion of the non-nucleotidic capture moiety-conjugated version of a nucleic acid probe species in the plurality of nucleic acid probe species falls within another range starting no lower than 5% and ending no higher than 100%.

In some embodiments, in the first iteration of the probe set, the first proportion of the non-nucleotidic capture moiety-conjugated version of each nucleic acid probe species in the plurality of nucleic acid probe species is at least 5%, at least 10%, at least 15%, at least 20%, at least 30%, at least 40%, at least 50%, at least 60%, at least 70%, at least 80%, at least 90%, or at least 95%. In some embodiments, the first proportion of the non-nucleotidic capture moiety-conjugated version of each nucleic acid probe species in the plurality of nucleic acid probe species is no more than 99%, no more than 95%, no more than 90%, no more than 80%, no more than 70%, no more than 60%, no more than 50%, no more than 40%, no more than 30%, or no more than 20%. In some embodiments, the first proportion of the non-nucleotidic capture moiety-conjugated version of each nucleic acid probe species in the plurality of nucleic acid probe species is from 5% to 95%, from 10% to 90%, from 20% to 80%, from 30% to 70%, from 40% to 60%, or from 45% to 55%. In some embodiments, the first proportion of the non-nucleotidic capture moiety-conjugated version of each nucleic acid probe species in the plurality of nucleic acid probe species is 100%. In some embodiments, the first proportion of the non-nucleotidic capture moiety-conjugated version of each nucleic acid probe species in the plurality of nucleic acid probe species falls within another range starting no lower than 5% and ending no higher than 100%.

In some embodiments, in the first iteration of the probe set, the second proportion of the capture moiety-free version of a nucleic acid probe species in the plurality of nucleic acid probe species is at least 1%, at least 5%, at least 10%, at least 15%, at least 20%, at least 30%, at least 40%, at least 50%, at least 60%, at least 70%, at least 80%, or at least 90%. In some embodiments, the second proportion of the capture moiety-free version of a nucleic acid probe species in the plurality of nucleic acid probe species is no more than 95%, no more than 90%, no more than 80%, no more than 70%, no more than 60%, no more than 50%, no more than 40%, no more than 30%, or no more than 20%. In some embodiments, the second proportion of the capture moiety-free version of a nucleic acid probe species in the plurality of nucleic acid probe species is from 1% to 95%, from 10% to 90%, from 20% to 80%, from 30% to 70%, from 40% to 60%, or from 45% to 55%. In some embodiments, the second proportion of the capture moiety-free version of a nucleic acid probe species in the plurality of nucleic acid probe species is zero. In some embodiments, the second proportion of the capture moiety-free version of a nucleic acid probe species in the plurality of nucleic acid probe species falls within another range starting no lower than 1% and ending no higher than 95%.

In some embodiments, in the first iteration of the probe set, the second proportion of the capture moiety-free version of each nucleic acid probe species in the plurality of nucleic acid probe species is at least 1%, at least 5%, at least 10%, at least 15%, at least 20%, at least 30%, at least 40%, at least 50%, at least 60%, at least 70%, at least 80%, or at least 90%. In some embodiments, the second proportion of the capture moiety-free version of each nucleic acid probe species in the plurality of nucleic acid probe species is no more than 95%, no more than 95%, no more than 90%, no more than 80%, no more than 70%, no more than 60%, no more than 50%, no more than 40%, no more than 30%, or no more than 20%. In some embodiments, the second proportion of the capture moiety-free version of each nucleic acid probe species in the plurality of nucleic acid probe species is from 1% to 95%, from 10% to 90%, from 20% to 80%, from 30% to 70%, from 40% to 60%, or from 45% to 55%. In some embodiments, the second proportion of the capture moiety-free version of each nucleic acid probe species in the plurality of nucleic acid probe species is zero. In some embodiments, the second proportion of the capture moiety-free version of each nucleic acid probe species in the plurality of nucleic acid probe species falls within another range starting no lower than 1% and ending no higher than 95%.

In some embodiments, each respective nucleic acid probe species corresponds to a plurality of nucleic acid probes in the nucleic acid probe set (e.g., having a first proportion of a non-nucleotidic capture moiety-conjugated version and a second proportion of a capture moiety-free version). In some embodiments, each respective nucleic acid probe species corresponds to at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, or at least 100 nucleic acid probes in the nucleic acid probe set. In some embodiments, each respective nucleic acid probe species corresponds to no more than 150, no more than 90, no more than 80, no more than 70, no more than 60, no more than 50, no more than 40, no more than 30, no more than 20, or no more than 10 nucleic acid probes in the nucleic acid probe set. In some embodiments, each respective nucleic acid probe species corresponds to from 3 to 5, from 3 to 10, from 10 to 50, from 10 to 100, from 10 to 20, from 15 to 75, from 5 to 20, from 20 to 90, or from 3 to 100 nucleic acid probes in the nucleic acid probe set. In some embodiments, each respective nucleic acid probe species falls within another range starting no lower than 3 pools and ending no higher than 150 nucleic acid probes.

Referring to Block 908, each nucleic acid probe species present in a respective pool, in the first plurality of pools, aligns to a portion of the genome that is at least 100 nucleotides away from any other portion of the genome that any other nucleic acid probe species present in a respective pool aligns with. In some embodiments, each nucleic acid probe species present in a respective pool, in the plurality of pools, aligns to a portion of the genome that is at least 200 nucleotides away, at least 300 nucleotides away, at least 400 nucleotides away, at least 500 nucleotides away, or at least 600 nucleotides away from any other portion of the genome that any other nucleic acid probe species present in a respective pool aligns with.

For instance, as described above, in some embodiments, the distribution of the plurality of nucleic acid probe species into the first plurality of pools is determined based on a threshold genomic distance between each nucleic acid probe species in the plurality of nucleic acid probe species, aligned to a reference genome. In some embodiments, each nucleic acid probe species present in a respective pool, in the first plurality of pools, aligns to a portion of the genome that is at least the threshold genomic distance away from any other portion of the genome that any other nucleic acid probe species present in the respective pool aligns with.

In some embodiments, the threshold genomic distance is at least 10, at least 25, at least 50, at least 75, at least 100, at least 200, at least 300, at least 400, at least 500, at least 600, at least 700, at least 800, at least 900, or at least 1000 nucleotides away. In some embodiments, the threshold genomic distance is from 10 to 40, from 20 to 200, from 100 to 500, from 100 to 1000, from 50 to 300, or from 100 to 200 nucleotides away.

Referring to Block 910, the method further includes analyzing the first iteration of the nucleic acid probe set against a first plurality of reference nucleic acid samples to obtain a corresponding recovery rate of each respective nucleic acid probe species in the plurality of nucleic acid probe species, where each respective pool in the first plurality of pools is analyzed in a separate reaction.

Advantageously, the methods for tuning a capture probe set described herein can account for one or more possible source of bias introduced during conventional targeted-probe sequencing reactions. For instance, in some embodiments, a workflow for such an assay includes steps of isolating nucleic acids from a test sample, generating a nucleic acid library from the isolated nucleic acids, amplifying the nucleic acid library, capturing targeted nucleic acids using a probe set (e.g., a balanced probe set as described herein), amplifying the captured nucleic acids, and then sequencing the amplified nucleic acids, described below in further detail. Each of these steps possibly introduces biases into the process. For instance, PCR amplification biases can be introduced both before and after capture of nucleic acids in this process. In some embodiments, the methods described herein leave out one or both of these leaving one or both of these amplification steps during the analysis of one or more test iterations of a probe set.

For instance, in some embodiments, analysis of a test iteration of a probe set includes contacting different aliquots of a reference nucleic acid sample with each pool of a test iteration of the probe set. Methods for isolating nucleic acids from biological samples are known in the art, and are dependent upon the type of nucleic acid being isolated (e.g., cfDNA, DNA, and/or RNA) and the type of sample from which the nucleic acids are being isolated (e.g., liquid biopsy samples, white blood cell buffy coat preparations, formalin-fixed paraffin-embedded (FFPE) solid tissue samples, and fresh frozen solid tissue samples). The selection of any particular nucleic acid isolation technique for use in conjunction with the embodiments described herein is well within the skill of the person having ordinary skill in the art, who will consider the sample type, the state of the sample, the type of nucleic acid being sequenced and the sequencing technology being used.

For instance, many techniques for DNA isolation, e.g., genomic DNA isolation, from a tissue sample are known in the art, such as organic extraction, silica adsorption, and anion exchange chromatography. Likewise, many techniques for RNA isolation, e.g., mRNA isolation, from a tissue sample are known in the art. For example, acid guanidinium thiocyanate-phenol-chloroform extraction (see, for example, Chomczynski and Sacchi, 2006, Nat Protoc, 1(2):581-85, which is hereby incorporated by reference herein), and silica bead/glass fiber adsorption (see, for example, Poeckh, T. et al., 2008, Anal Biochem., 373(2):253-62, which is hereby incorporated by reference herein). The selection of any particular DNA or RNA isolation technique for use in conjunction with the embodiments described herein is well within the skill of the person having ordinary skill in the art, who will consider the tissue type, the state of the tissue, e.g., fresh, frozen, formalin-fixed, paraffin-embedded (FFPE), and the type of nucleic acid analysis that is to be performed.

In some embodiments where the biological sample is a liquid biopsy sample, e.g., a blood or blood plasma sample, cfDNA is isolated from blood samples using commercially available reagents, including proteinase K, to generate a liquid solution of cfDNA.

In some embodiments, the reference nucleic acid samples have been preparing as a nucleic acid library from the isolated nucleic acids (e.g., cfDNA, DNA, and/or RNA). For example, in some embodiments, DNA libraries (e.g., gDNA and/or cfDNA libraries) are prepared from isolated DNA from the one or more biological samples. In some embodiments, the DNA libraries are prepared using a commercial library preparation kit, e.g., the KAPA Hyper Prep Kit, a New England Biolabs (NEB) kit, or a similar kit.

In some embodiments, isolated nucleic acids are mechanically sheared to an average length using an ultrasonicator. In some embodiments, isolated nucleic acid molecules are analyzed to determine their fragment size, e.g., through gel electrophoresis techniques and/or the use of a device such as a LabChip GX Touch. The skilled artisan will know of an appropriate range of fragment sizes, based on the sequencing technique being employed, as different sequencing techniques have differing fragment size requirements for robust sequencing. In some embodiments, quality control testing is performed on the extracted nucleic acids (e.g., DNA and/or RNA), e.g., to assess the nucleic acid concentration and/or fragment size. For example, sizing of DNA fragments provides valuable information used for downstream processing, such as determining whether DNA fragments require additional shearing prior to sequencing.

In some embodiments, during library preparation, adapters (e.g., UDI adapters, such as Roche SeqCap dual end adapters, or UMI adapters such as full length or stubby Y adapters) are ligated onto the nucleic acid molecules. In some embodiments, the adapters include unique molecular identifiers (UMIs), which are short nucleic acid sequences (e.g., 3-10 base pairs) that are added to ends of DNA fragments during adapter ligation. In some embodiments, UMIs are degenerate base pairs that serve as a unique tag that can be used to identify sequence reads originating from a specific DNA fragment. In some embodiments, e.g., when multiplex sequencing will be used to sequence DNA from a plurality of samples (e.g., from the same or different subjects) in a single sequencing reaction, a patient-specific index is also added to the nucleic acid molecules. In some embodiments, the patient specific index is a short nucleic acid sequence (e.g., 3-20 nucleotides) that are added to ends of DNA fragments during library construction, that serve as a unique tag that can be used to identify sequence reads originating from a specific patient sample. Examples of identifier sequences are described, for example, in Kivioja et al., Nat. Methods 9(1):72-74 (2011) and Islam et al., Nat. Methods 11(2):163-66 (2014), the contents of which are hereby incorporated by reference, in their entireties, for all purposes.

In some embodiments, an adapter includes a PCR primer landing site, designed for efficient binding of a PCR or second-strand synthesis primer used during the sequencing reaction. In some embodiments, an adapter includes an anchor binding site, to facilitate binding of the DNA molecule to anchor oligonucleotide molecules on a sequencer flow cell, serving as a seed for the sequencing process by providing a starting point for the sequencing reaction. During PCR amplification following adapter ligation, the UMIs, patient indexes, and binding sites are replicated along with the attached DNA fragment. This provides a way to identify sequence reads that came from the same original fragment in downstream analysis.

In some embodiments, DNA libraries (e.g., DNA or cDNA libraries) are amplified and purified using commercial reagents, (e.g., Axygen MAG PCR clean up beads). In some such embodiments, the concentration and/or quantity of the DNA molecules are then quantified using a fluorescent dye and a fluorescence microplate reader, standard spectrofluorometer, or filter fluorometer. In some embodiments, library amplification is performed on a device (e.g., an Illumina C-Bot2) and the resulting flow cell containing amplified target-captured DNA libraries is sequenced on a next generation sequencer (e.g., an Illumina HiSeq 4000 or an Illumina NovaSeq 6000) to a unique on-target depth selected by the user. In some embodiments, DNA library preparation is performed with an automated system, using a liquid handling robot (e.g., a SciClone NGSx). In some embodiments, DNA libraries are not amplified prior to probe capture, in order to eliminate amplification biases introduced by such an amplification step.

In some embodiments, the nucleic acids captured by the probes in the test iteration of the probe set are then further amplified, to improve subsequent nucleic acid sequencing. However, in other embodiments, the nucleic acids captured by the probes in the test iteration of the probe set are sequenced without being further amplified, in order to eliminate amplification biases introduced by such an amplification step.

Accordingly, many different variations of this particular analysis methodology, accounting for different combinations of bias, can be used in conjunction with the methods described herein. For example, in some embodiments, a reference nucleic acid library that has not been amplified is contacted with a test iteration of a probe set, as described herein, and the captured nucleic acids are sequenced without further amplification. When performed using the pooled methodology described herein, this methodology essentially tunes only for the binding kinetics of the probe species. When performed using a bulk methodology, where all of the probe species are used in a single capture reaction, this methodology tunes for the binding kinetics of the probe species accounting for neighboring probe effects.

In another embodiment, an analysis step includes contacting a reference nucleic acid library that has been amplified with a test iteration of a probe set, as described herein, and sequencing the captured nucleic acids without further amplification. When performed using the pooled methodology described herein, this methodology tunes for the binding kinetics of the probe species accounting for pre-capture amplification bias. When performed using a bulk methodology, where all of the probe species are used in a single capture reaction, this methodology tunes for the binding kinetics of the probe species accounting for neighboring probe effects and pre-capture amplification bias.

In another embodiment, an analysis step includes contacting a reference nucleic acid library that has not been amplified with a test iteration of a probe set, as described herein, amplifying the captured nucleic acids, and then sequencing the amplified nucleic acids. When performed using the pooled methodology described herein, this methodology tunes for the binding kinetics of the probe species accounting for post-capture amplification bias. When performed using a bulk methodology, where all of the probe species are used in a single capture reaction, this methodology tunes for the binding kinetics of the probe species accounting for neighboring probe effects and post-capture amplification bias.

In another embodiment, an analysis step includes contacting a reference nucleic acid library that has been amplified with a test iteration of a probe set, as described herein, amplifying the captured nucleic acids, and then sequencing the amplified nucleic acids. When performed using the pooled methodology described herein, this methodology tunes for the binding kinetics of the probe species accounting for pre-capture and post-capture amplification bias. When performed using a bulk methodology, where all of the probe species are used in a single capture reaction, this methodology tunes for the binding kinetics of the probe species accounting for neighboring probe effects, as well as pre-capture and post-capture amplification bias.

In some embodiments, different combinations of these methodologies are used to analyze a single test iteration of a probe set or different test iterations of a probe set. For example, in some embodiments, a first test iteration of the probe set is analyzed using a first methodology and a second iteration of the probe set is analyzed using a second methodology.

In some embodiments, the analyzing is performed using any suitable experimental design (e.g., sequencing assay design), as will be apparent to one skilled in the art. For example, in some embodiments, the analyzing is performed on a sequencing library prepared using enrichment of target genomic loci via capture probes (e.g., the first proportion of a non-nucleotidic capture moiety-conjugated version for each respective nucleic acid probe species in a plurality of nucleic acid probe species). In some embodiments, the analyzing is performed using no amplification, pre-capture amplification, post-capture amplification, or both pre-capture amplification and post-capture amplification.

For instance, in some embodiments, the analyzing comprises, for each respective pool in the plurality of pools, capturing, for each respective reference nucleic acid sample in the plurality of reference samples, nucleic acids from the respective reference nucleic acid sample using the respective pool; measuring, for each respective nucleic acid probe species present in the respective pool, a respective recovery rate for each respective reference nucleic acid sample in the plurality of nucleic acid reference samples, thereby obtaining a corresponding plurality of respective recovery rates for the respective nucleic acid probe species; and determining, for each respective nucleic acid probe species present in the respective pool, the corresponding recovery rate for the respective nucleic acid probe species based on the corresponding plurality of respective recovery rates.

As an example, in some embodiments, each respective pool in the first plurality of pools comprises nucleic acid probe species having non-overlapping nucleic acid probe sequences. In some such embodiments, the analyzing comprises using the recovery rate to balance the probe set based on the relative capture efficiencies of each respective probe species in the plurality of nucleic acid probe species.

In some embodiments, each respective pool in the first plurality of pools comprises nucleic acid probe species having overlapping nucleic acid probe sequences, and the analyzing further comprises determining a tuning rate accounting for overlapping nucleic acid probe effects. In some such embodiments, the analyzing comprises using the recovery rate to balance the probe set based on the relative capture efficiencies of each respective probe species in the plurality of nucleic acid probe species and the tuning rate for overlapping nucleic acid probe effects.

In some embodiments, each respective pool in the first plurality of pools comprises nucleic acid probe species having neighboring (e.g., adjacent) nucleic acid probe sequences, and the analyzing further comprises determining a tuning rate accounting for neighboring nucleic acid probe effects. In some such embodiments, the analyzing comprises using the recovery rate to balance the probe set based on the relative capture efficiencies of each respective probe species in the plurality of nucleic acid probe species and the tuning rate for neighboring nucleic acid probe effects.

In some embodiments, the analyzing comprises, for each respective pool in the plurality of pools, capturing, for each respective reference nucleic acid sample in the plurality of reference samples, nucleic acids from the respective reference nucleic acid sample using the respective pool; amplifying, for each respective reference nucleic acid sample in the plurality of reference samples, the captured nucleic acids from the respective reference nucleic acid sample; measuring, for each respective nucleic acid probe species present in the respective pool, a respective recovery rate for each respective reference nucleic acid sample in the plurality of nucleic acid reference samples based on the amplified nucleic acids, thereby obtaining a corresponding plurality of respective recovery rates for the respective nucleic acid probe species; and determining, for each respective nucleic acid probe species present in the respective pool, the corresponding recovery rate for the respective nucleic acid probe species based on the corresponding plurality of respective recovery rates.

In some embodiments, each respective pool comprises nucleic acid probe species having non-overlapping nucleic acid probe sequences. In some such embodiments, the analyzing comprises using the plurality of respective recovery rates to balance the probe set based on the relative capture efficiencies of each respective probe species in the plurality of nucleic acid probe species and post-capture amplification bias.

In some embodiments, each respective pool comprises nucleic acid probe species having overlapping nucleic acid probe sequences and the analyzing further comprises determining a tuning rate accounting for overlapping nucleic acid probe effects. In some such embodiments, the analyzing comprises using the plurality of respective recovery rates to balance the probe set based on the relative capture efficiencies of each respective probe species in the plurality of nucleic acid probe species, post-capture amplification bias, and the tuning rate for overlapping nucleic acid probe effects.

In some embodiments, each respective pool comprises nucleic acid probe species having neighboring (e.g., adjacent) nucleic acid probe sequences and the analyzing further comprises determining a tuning rate accounting for neighboring nucleic acid probe effects. In some such embodiments, the analyzing comprises using the plurality of respective recovery rates to balance the probe set based on the relative capture efficiencies of each respective probe species in the plurality of nucleic acid probe species, post-capture amplification bias, and the tuning rate for neighboring nucleic acid probe effects.

In some embodiments, the analyzing comprises, for each respective pool in the plurality of pools, amplifying nucleic acids in the respective pool; capturing, for each respective reference nucleic acid sample in the plurality of reference samples, amplified nucleic acids from the respective reference nucleic acid sample using the respective pool; measuring, for each respective nucleic acid probe species present in the respective pool, a respective recovery rate for each respective reference nucleic acid sample in the plurality of nucleic acid reference samples based on the captured nucleic acids, thereby obtaining a corresponding plurality of respective recovery rates for the respective nucleic acid probe species; and determining, for each respective nucleic acid probe species present in the respective pool, the corresponding recovery rate for the respective nucleic acid probe species based on the corresponding plurality of respective recovery rates.

In some embodiments, each respective pool comprises nucleic acid probe species having non-overlapping nucleic acid probe sequences. In some such embodiments, the analyzing comprises using the plurality of respective recovery rates to balance the probe set based on the relative capture efficiencies of each respective probe species in the plurality of nucleic acid probe species and pre-capture amplification bias.

In some embodiments, each respective pool comprises nucleic acid probe species having overlapping nucleic acid probe sequences and the analyzing further comprises determining a tuning rate accounting for overlapping nucleic acid probe effects. In some such embodiments, the analyzing comprises using the plurality of respective recovery rates to balance the probe set based on the relative capture efficiencies of each respective probe species in the plurality of nucleic acid probe species, pre-capture amplification bias, and the tuning rate for overlapping nucleic acid probe effects.

In some embodiments, each respective pool comprises nucleic acid probe species having neighboring (e.g., adjacent) nucleic acid probe sequences and the analyzing further comprises determining a tuning rate accounting for neighboring nucleic acid probe effects. In some such embodiments, the analyzing comprises using the plurality of respective recovery rates to balance the probe set based on the relative capture efficiencies of each respective probe species in the plurality of nucleic acid probe species, pre-capture amplification bias, and the tuning rate for neighboring nucleic acid probe effects.

In some embodiments, the analyzing comprises, for each respective pool in the plurality of pools, amplifying nucleic acids in the respective pool; capturing, for each respective reference nucleic acid sample in the plurality of reference samples, amplified nucleic acids from the respective reference nucleic acid sample using the respective pool; amplifying, for each respective reference nucleic acid sample in the plurality of reference samples, the captured nucleic acids from the respective reference nucleic acid sample; measuring, for each respective nucleic acid probe species present in the respective pool, a respective recovery rate for each respective reference nucleic acid sample in the plurality of nucleic acid reference samples based on the amplified nucleic acids, thereby obtaining a corresponding plurality of respective recovery rates for the respective nucleic acid probe species; and determining, for each respective nucleic acid probe species present in the respective pool, the corresponding recovery rate for the respective nucleic acid probe species based on the corresponding plurality of respective recovery rates.

In some embodiments, each respective pool comprises nucleic acid probe species having non-overlapping nucleic acid probe sequences. In some such embodiments, the analyzing comprises using the plurality of respective recovery rates to balance the probe set based on the relative capture efficiencies of each respective probe species in the plurality of nucleic acid probe species, pre-capture amplification bias, and post-capture amplification bias.

In some embodiments, each respective pool comprises nucleic acid probe species having overlapping nucleic acid probe sequences and the analyzing further comprises determining a tuning rate accounting for overlapping nucleic acid probe effects. In some such embodiments, the analyzing comprises using the plurality of respective recovery rates to balance the probe set based on the relative capture efficiencies of each respective probe species in the plurality of nucleic acid probe species, pre-capture amplification bias, post-capture amplification bias, and the tuning rate for overlapping nucleic acid probe effects.

In some embodiments, each respective pool comprises nucleic acid probe species having neighboring (e.g., adjacent) nucleic acid probe sequences and the analyzing further comprises determining a tuning rate accounting for neighboring nucleic acid probe effects. In some such embodiments, the analyzing comprises using the plurality of respective recovery rates to balance the probe set based on the relative capture efficiencies of each respective probe species in the plurality of nucleic acid probe species, pre-capture amplification bias, post-capture amplification bias, and the tuning rate for neighboring nucleic acid probe effects.

In some embodiments, the method further comprises determining a tuning rate accounting for sequencing bias. In some embodiments, the analyzing comprises using the plurality of respective recovery rates to balance the probe set based at least in part on the tuning rate for sequencing bias.

In some embodiments, the recovery rate of a respective nucleic acid probe species is determined by sequencing the captured or amplified nucleic acids and quantitating the number of raw sequence reads from the sequencing that overlap the respective nucleic acid probe by a minimum number of nucleic acids.

In some embodiments, the recovery rate of a respective nucleic acid probe species is determined by sequencing the captured or amplified nucleic acids, de-duplicating raw sequence reads from the sequencing to generate unique sequence reads, and quantitating the number of unique sequence reads that overlap the respective nucleic acid probe by a minimum number of nucleic acids.

For example, in some embodiments, the minimum number of nucleic acids is at least 2, at least 3, at least 4, at least 5, at least 10, at least 25, at least 50, at least 75, or at least 100 nucleic acids.

In some embodiments, the corresponding recovery rate for the respective nucleic acid probe species is a measure of central tendency for some or all of the corresponding plurality of respective recovery rates (e.g., for each respective reference nucleic acid sample in the plurality of nucleic acid reference samples). Non-limiting examples of measures of central tendency include an arithmetic mean, weighted mean, midrange, midhinge, trimean, geometric mean, geometric median, Winsorized mean, median, and mode.

In some embodiments, the corresponding recovery rate for the respective nucleic acid probe species is obtained using a subset of the plurality of respective recovery rates (e.g., comprising each respective recovery rate for each respective reference nucleic acid sample in the plurality of nucleic acid reference samples). For instance, in some embodiments, the corresponding recovery rate for a respective nucleic acid probe species is obtained by excluding a first percentage of the highest recovery rates and a second percentage of the lowest recovery rates obtained using the plurality of nucleic acid reference samples for the respective nucleic acid probe species, and determining an average of the remaining recovery rates.

In some embodiments, the first percentage of the highest recovery rates is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, or more than 20%. In some embodiments, the second percentage of the lowest recovery rates is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, or more than 20%.

In some embodiments, a plurality (e.g., the first plurality) of reference nucleic acid samples used for measuring the respective recovery rates for each respective nucleic acid probe species comprises at least 5, at least 10, at least 15, at least 25, at least 30, at least 40, at least 50, at least 100, at least 200, at least 250, at least 400, at least 500, at least 600, at least 700, at least 800, at least 900, at least 1000, at least 2000, at least 2500, at least 4000, at least 5000, at least 6000, at least 7000, at least 8000, at least 9000, or at least 10,000 reference nucleic acid samples. In some embodiments, the plurality of reference nucleic acid samples comprises no more than 15,000, no more than 10,000, no more than 7500, no more than 5000, no more than 4000, no more than 3000, no more than 2000, no more than 1000, no more than 750, no more than 500, no more than 250, no more than 100, no more than 50, or no more than 25 reference nucleic acid samples. In some embodiments, the plurality of reference nucleic acid samples comprises from 10 to 50, from 25 to 100, from 100 to 500, from 100 to 1000, from 1000 to 2000, from 10 to 500, from 500 to 2000, from 1000 to 5000, from 5000 to 10,000, or from 10,000 to 15,000 reference nucleic acid samples. In some embodiments, the plurality of reference nucleic acid samples falls within another range starting no lower than 5 samples and ending no higher than 15,000 samples.

Referring to Block 912, the method further includes identifying, based on the corresponding recovery rate of each respective nucleic acid probe species, a first subset of the plurality of nucleic acid probe species that does not satisfy a minimum recovery rate threshold, and a second subset of the plurality of nucleic acid probe species that does not satisfy a maximum recovery rate threshold.

In some embodiments, the minimum recovery threshold and maximum recovery threshold for the respective nucleic acid probe species is determined by a comparison between some or all of the corresponding plurality of respective recovery rates for the plurality of nucleic acid probe species in the respective pool that contains the respective nucleic acid probe species. In some embodiments, the minimum recovery threshold and maximum recovery threshold for the respective nucleic acid probe species is determined by a comparison between some or all of the corresponding plurality of respective recovery rates for the plurality of nucleic acid probe species in the nucleic acid probe set.

In some embodiments, the minimum recovery threshold and maximum recovery threshold are set relative to a measure of central tendency for the recovery rate of all of the nucleic acid probe species (e.g., in a respective pool and/or in a respective nucleic acid probe set). Non-limiting examples of measures of central tendency include an arithmetic mean, weighted mean, midrange, midhinge, trimean, geometric mean, geometric median, Winsorized mean, median, and mode.

In some embodiments, the minimum recovery threshold is no more than 10% less than the measure of central tendency for the recovery rate of all of the nucleic acid probe species. In some embodiments, the minimum recovery threshold is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% less than the measure of central tendency for the recovery rate of all of the nucleic acid probe species. In some embodiments, the minimum recovery threshold is at least 5%, at least 10%, at least 20%, at least 30%, at least 40%, or at least 45% less than the measure of central tendency for the recovery rate of all of the nucleic acid probe species.

In some embodiments, the maximum recovery threshold is no more than 10% greater than the measure of central tendency for the recovery rate of all of the nucleic acid probe species. In some embodiments, the maximum recovery threshold is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% greater than the measure of central tendency for the recovery rate of all of the nucleic acid probe species. In some embodiments, the maximum recovery threshold is at least 5%, at least 10%, at least 20%, at least 30%, at least 40%, or at least 45% greater than the measure of central tendency for the recovery rate of all of the nucleic acid probe species.

In some embodiments, the minimum recovery threshold is the measure of central tendency for the recovery rate of all of the nucleic acid probe species. In some embodiments, the maximum recovery threshold is the measure of central tendency for the recovery rate of all of the nucleic acid probe species.

For example, in some embodiments, each nucleic acid probe species in the nucleic acid probe set satisfies a minimum recovery threshold and a maximum recovery threshold when each nucleic acid probe species in the probe set is exactly balanced across the plurality of nucleic acid probe species in the pool and/or the nucleic acid probe set.

Referring to Block 914, the method further includes using the identification of the first and second subsets of the plurality of nucleic acid probe species to make a first adjustment to respective proportions of (i) non-nucleotidic capture moiety conjugated versions and (ii) capture moiety-free versions of the respective nucleic acid probe species in a final design for the nucleic acid probe set, thereby establishing a first adjusted version of the final design for the nucleic acid probe set.

For instance, referring to Blocks 916-918, the proportion of non-nucleotidic capture moiety conjugated versions of respective nucleic acid probe species in the first subset of the plurality of nucleic acid probe species are adjusted upwards in the final design for the nucleic acid probe set by the first adjustment, and the proportion of non-nucleotidic capture moiety conjugated versions of respective nucleic acid probe species in the second subset of the plurality of nucleic acid probe species are adjusted downwards in the final design for the nucleic acid probe set by the first adjustment.

In some embodiments, the adjustment comprises increasing or decreasing the proportion of biotinylated capture probes in a plurality of capture probes for a target locus.

In some embodiments, the adjustment increases the proportion of the non-nucleotidic capture moiety-conjugated version (e.g., the proportion of biotinylated capture probes) of each nucleic acid probe species in the plurality of nucleic acid probe species identified as failing to satisfy a minimum recovery threshold (e.g., low performing nucleic acid probe species that are poorly detected).

In some embodiments, the adjustment decreases the proportion of the non-nucleotidic capture moiety-conjugated version (e.g., the proportion of biotinylated capture probes) of each nucleic acid probe species in the plurality of nucleic acid probe species identified as failing to satisfy a maximum recovery threshold (e.g., high performing nucleic acid probe species that overrepresented). See, for instance, FIGS. 8C-8D and Example 2 below.

In some embodiments, for each respective nucleic acid probe species in the identified first (e.g., low performing nucleic acid probe species) and second (e.g., high performing nucleic acid probe species) subsets, the adjustment is determined based upon the difference between the corresponding recovery rate of the respective nucleic acid probe species and a measure of central tendency for the recovery rate of all of the nucleic acid probe species in the respective pool and/or the nucleic acid probe set. For example, as illustrated in FIG. 8C and discussed in Example 2 below, in some embodiments, the adjustment is a correction to the mean recovery rate of all the nucleic acid probe species in the respective nucleic acid probe set.

In some embodiments, for each respective nucleic acid probe species in the identified first and second subsets, the adjustment is proportional to the difference between the corresponding recovery rate of the respective nucleic acid probe species and a measure of central tendency for the recovery rate of all of the nucleic acid probe species in the respective pool and/or the nucleic acid probe set.

In some embodiments, for each respective nucleic acid probe species in the identified first and second subsets, the adjustment is based upon the initial proportion of the non-nucleotidic capture moiety-conjugated version of the respective nucleic acid probe species. For example, in some embodiments, an adjustment for a respective capture probe may be limited by a maximum biotinylation level (e.g., 100%), such that a full correction for the difference in coverage for the respective probe and the mean will not be achieved before the maximum number of biotinylated capture probes is reached. For example, FIG. 8C indicates that a 556% increase in capture activity is required to correct a particular probe (P011) to the mean recovery rate. Such an increase in the proportion of biotinylated capture probes is feasible only if the current proportion of biotinylated capture probes is less than 20%.

Referring to Block 920, in some embodiments, the method further comprises obtaining a second iteration of the nucleic acid probe set comprising the plurality of nucleic acid probe species distributed in a second plurality of pools.

In some embodiments, the molarity of each nucleic acid probe species in the plurality of nucleic acid probe species is the same in the second iteration of the probe set. In some embodiments, the relative molarity of each nucleic acid probe species in the plurality of nucleic acid probe species in the second iteration of the probe set is the same as in the first iteration of the probe set.

Referring to Blocks 922-924, each respective nucleic acid probe species is present in the second iteration of the nucleic acid probe set as (i) a respective third proportion of a non-nucleotidic capture moiety conjugated version of the respective nucleic acid probe species, and (ii) a respective fourth proportion of a capture moiety-free version of the respective nucleic acid probe species, based on the first adjusted version of the final design for the nucleic acid probe set. Each nucleic acid probe species present in a respective pool, in the second plurality of pools, aligns to a portion of the genome that is at least 100 nucleotides away from any other portion of the genome that any other nucleic acid probe species present in a respective pool aligns with.

Referring to Block 926, in some embodiments, the method further comprises analyzing the second iteration of the nucleic acid probe set against a second plurality of reference nucleic acid samples to obtain a corresponding recovery rate of each respective nucleic acid probe species in the plurality of nucleic acid probe species, where each respective pool in the second plurality of pools is analyzed in a separate reaction.

In some embodiments, the analyzing comprises, for each respective pool in the plurality of pools, capturing, for each respective reference nucleic acid sample in the plurality of reference samples, nucleic acids from the respective reference nucleic acid sample using the respective pool; measuring, for each respective nucleic acid probe species present in the respective pool, a respective recovery rate for each respective reference nucleic acid sample in the plurality of nucleic acid reference sample, thereby obtaining a corresponding plurality of respective recovery rates for the respective nucleic acid probe species; and determining, for each respective nucleic acid probe species present in the respective pool, the corresponding recovery rate for the respective nucleic acid probe species based on the corresponding plurality of respective recovery rates.

In some embodiments, the analyzing comprises, for each respective pool in the plurality of pools, capturing, for each respective reference nucleic acid sample in the plurality of reference samples, nucleic acids from the respective reference nucleic acid sample using the respective pool; amplifying, for each respective reference nucleic acid sample in the plurality of reference samples, the captured nucleic acids from the respective reference nucleic acid sample; measuring, for each respective nucleic acid probe species present in the respective pool, a respective recovery rate for each respective reference nucleic acid sample in the plurality of nucleic acid reference sample based on the amplified nucleic acids, thereby obtaining a corresponding plurality of respective recovery rates for the respective nucleic acid probe species; and determining, for each respective nucleic acid probe species present in the respective pool, the corresponding recovery rate for the respective nucleic acid probe species based on the corresponding plurality of respective recovery rates.

In some embodiments, the analyzing comprises, for each respective pool in the plurality of pools, amplifying nucleic acids in the respective pool; capturing, for each respective reference nucleic acid sample in the plurality of reference samples, amplified nucleic acids from the respective reference nucleic acid sample using the respective pool; measuring, for each respective nucleic acid probe species present in the respective pool, a respective recovery rate for each respective reference nucleic acid sample in the plurality of nucleic acid reference sample based on the captured nucleic acids, thereby obtaining a corresponding plurality of respective recovery rates for the respective nucleic acid probe species; and determining, for each respective nucleic acid probe species present in the respective pool, the corresponding recovery rate for the respective nucleic acid probe species based on the corresponding plurality of respective recovery rates.

In some embodiments, the analyzing comprises, for each respective pool in the plurality of pools, amplifying nucleic acids in the respective pool; capturing, for each respective reference nucleic acid sample in the plurality of reference samples, amplified nucleic acids from the respective reference nucleic acid sample using the respective pool; amplifying, for each respective reference nucleic acid sample in the plurality of reference samples, the captured nucleic acids from the respective reference nucleic acid sample; measuring, for each respective nucleic acid probe species present in the respective pool, a respective recovery rate for each respective reference nucleic acid sample in the plurality of nucleic acid reference sample based on the amplified nucleic acids, thereby obtaining a corresponding plurality of respective recovery rates for the respective nucleic acid probe species; and determining, for each respective nucleic acid probe species present in the respective pool, the corresponding recovery rate for the respective nucleic acid probe species based on the corresponding plurality of respective recovery rates.

In some embodiments, different iterations can use different experimental design conditions (e.g., different methods for analyzing including pre-capture amplification and/or post-capture amplification). In some embodiments, a first iteration and any subsequent iteration can use the same or different conditions for analysis. In some embodiments, the analysis comprises any of the experimental design conditions described above.

In some embodiments, the method comprises comparing a first measure of the distribution of the recovery rates for all of the nucleic acid probe species in the first iteration of the nucleic acid probe set to a second measure of the distribution of the recovery rates for all of the nucleic acid probe species in the second iteration of the nucleic acid probe set. Measures of distribution include, but are not limited to, variance, standard deviation, and/or standard error.

In some embodiments, a first measure of the distribution (e.g., a standard deviation) of the recovery rates for all of the nucleic acid probe species in the second iteration of the nucleic acid probe set is at least 25% smaller than a second measure of the distribution of the recovery rates for all of the nucleic acid probe species in the first iteration of the nucleic acid probe set. In some embodiments, a first measure of the distribution (e.g., a standard deviation) of the recovery rates for all of the nucleic acid probe species in the second iteration of the nucleic acid probe set is at least 5%, at least 10%, at least 15%, at least 20%, at least 25%, least 30%, at least 40%, at least 50%, at least 60%, at least 70%, at least 80%, or at least 90% smaller than a second measure of the distribution of the recovery rates for all of the nucleic acid probe species in the first iteration of the nucleic acid probe set.

In some embodiments, the method comprises repeating the obtaining, analyzing, identifying, and adjusting for a plurality of iterations. In some such embodiments, the method comprises comparing a third measure of the distribution of the recovery rates for all of the nucleic acid probe species in a third iteration of the nucleic acid probe set to the second measure of the distribution of the recovery rates for all of the nucleic acid probe species in the second iteration of the nucleic acid probe set.

For example, in some embodiments, a third measure of the distribution (e.g., a standard deviation) of the recovery rates for all of the nucleic acid probe species in the third iteration of the nucleic acid probe set is at least 10% smaller than the second measure of the distribution of the recovery rates for all of the nucleic acid probe species in the second iteration of the nucleic acid probe set. In some embodiments, a third measure of the distribution (e.g., a standard deviation) of the recovery rates for all of the nucleic acid probe species in the third iteration of the nucleic acid probe set is at least 5%, at least 10%, at least 15%, at least 20%, at least 25%, least 30%, at least 40%, at least 50%, at least 60%, at least 70%, at least 80%, or at least 90% smaller than the second measure of the distribution of the recovery rates for all of the nucleic acid probe species in the second iteration of the nucleic acid probe set.

Referring to Block 928, the method further includes identifying, based on the corresponding recovery rate of each respective nucleic acid probe species, a third subset of the plurality of nucleic acid probe species that does not satisfy a minimum recovery rate threshold, and a fourth subset of the plurality of nucleic acid probe species that does not satisfy a maximum recovery rate threshold.

Referring to Blocks 930-934, the method further includes using the identification of the third and fourth subsets of the plurality of nucleic acid probe species to make a second adjustment to respective proportions of (i) non-nucleotidic capture moiety conjugated versions and (ii) capture moiety-free versions of the respective nucleic acid probe species in the final design for the nucleic acid probe set, thereby establishing a second adjusted version of the final design for the nucleic acid probe set. The proportion of non-nucleotidic capture moiety conjugated versions of respective nucleic acid probe species in the third subset of the plurality of nucleic acid probe species are adjusted upwards in the final design for the nucleic acid probe set by the second adjustment, and the proportion of non-nucleotidic capture moiety conjugated versions of respective nucleic acid probe species in the second subset of the plurality of nucleic acid probe species are adjusted downwards in the final design for the nucleic acid probe set by the first adjustment.

In some embodiments, the method further comprises, for each respective iteration in a plurality of iterations, repeating the obtaining the respective iteration of the nucleic acid probe set comprising the plurality of nucleic acid probe species distributed in a corresponding respective plurality of pools. Each respective nucleic acid probe species is present in the respective iteration of the nucleic acid probe set as (i) a respective updated proportion of a non-nucleotidic capture moiety conjugated version of the respective nucleic acid probe species, and (ii) a respective updated proportion of a capture moiety-free version of the respective nucleic acid probe species, where the updated proportion (i) and the updated proportion (ii) are based on an adjusted version of the final design for the nucleic acid probe set (e.g., from a previous iteration). Each nucleic acid probe species present in a respective pool, in the corresponding respective plurality of pools, aligns to a portion of the genome that is at least 100 nucleotides away from any other portion of the genome that any other nucleic acid probe species present in a respective pool aligns with.

In some such embodiments, the method comprises, for each respective iteration in a plurality of iterations, analyzing the respective iteration of the nucleic acid probe set against a corresponding respective plurality of reference nucleic acid samples to obtain a corresponding recovery rate of each respective nucleic acid probe species in the plurality of nucleic acid probe species, where each respective pool in the respective plurality of pools is analyzed in a separate reaction. The method further includes identifying, based on the corresponding recovery rate of each respective nucleic acid probe species, a subset of the plurality of nucleic acid probe species that does not satisfy a minimum recovery rate threshold, and a subset of the plurality of nucleic acid probe species that does not satisfy a maximum recovery rate threshold.

In some such embodiments, the method further comprises, for each respective iteration in a plurality of iterations, using the identification of the subsets of the plurality of nucleic acid probe species that fail to satisfy the minimum and maximum recovery rate thresholds to make an adjustment to respective proportions of (i) non-nucleotidic capture moiety conjugated versions and (ii) capture moiety-free versions of the respective nucleic acid probe species in the final design for the nucleic acid probe set, thereby establishing a corresponding respective adjusted version of the final design for the nucleic acid probe set. The proportion of non-nucleotidic capture moiety conjugated versions of respective nucleic acid probe species in the subset of the plurality of nucleic acid probe species that does not satisfy a minimum recovery rate threshold are adjusted upwards in the final design for the nucleic acid probe set by the second adjustment, and the proportion of non-nucleotidic capture moiety conjugated versions of respective nucleic acid probe species in the subset of the plurality of nucleic acid probe species that does not satisfy a maximum recovery rate threshold are adjusted downwards in the final design for the nucleic acid probe set by the first adjustment.

In some embodiments, the plurality of iterations is at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 200, at least 300, at least 400, or at least 500. In some embodiments, the plurality of iterations is from 2 iterations to 500 iterations, from 5 iterations to 500 iterations, from 10 iterations to 500 iterations, from 25 iterations to 500 iterations, from 50 iterations to 500 iterations, from 100 iterations to 500 iterations, or from 250 iterations to 500 iterations. In some embodiments, the plurality of iterations is from 2 iterations to 250 iterations, from 5 iterations to 250 iterations, from 10 iterations to 250 iterations, from 25 iterations to 250 iterations, from 50 iterations to 250 iterations, or from 100 iterations to 250 iterations. In some embodiments, the plurality of iterations is from 2 iterations to 100 iterations, from 5 iterations to 100 iterations, from 10 iterations to 100 iterations, from 25 iterations to 100 iterations, or from 50 iterations to 100 iterations. In some embodiments, the plurality of iterations is from 2 iterations to 50 iterations, from 5 iterations to 50 iterations, from 10 iterations to 50 iterations, or from 25 iterations to 50 iterations. In some embodiments, the plurality of iterations is from 2 iterations to 25 iterations, from 5 iterations to 25 iterations, or from 10 iterations to 25 iterations. In some embodiments, the plurality of iterations is from 2 iterations to 10 iterations, from 5 iterations to 10 iterations, or from 2 iterations to 5 iterations.

In some embodiments, the method is repeated until a respective measure of the distribution (e.g., a standard deviation) of the recovery rates for all of the nucleic acid probe species in a respective iteration satisfies a distribution threshold. In some embodiments, the distribution threshold is a particular standard deviation, or other metric of distribution, for the recovery rates for all of the nucleic acid probe species. In some embodiments, the distribution threshold is a requirement that a certain percentage of the recovery rates (e.g., at least 75%, 80%, 85%, 90%, 95%, 98%, 99%, 99.5%, 99.9%, or 100%) fall within a range around a measure of central tendency (e.g., mean or median) for the recovery rates for all of the nucleic acid probe species. In some embodiments, the range is no more than 1% difference from the measure of central tendency. In some embodiments, the range is not more than 2%, no more than 2.5%, no more than 5%, or no more than 10% difference from the measure of central tendency.

In some embodiments, the method is repeated until no nucleic acid probe species in the plurality of nucleic acid probe species fail to satisfy a minimum recovery rate threshold (e.g., all nucleic acid probe species in the plurality of nucleic acid probe species satisfy the minimum recovery rate threshold).

In some embodiments, the method is repeated until no nucleic acid probe species in the plurality of nucleic acid probe species fail to satisfy a maximum recovery rate threshold (e.g., all nucleic acid probe species in the plurality of nucleic acid probe species satisfy the maximum recovery rate threshold).

In some embodiments, the method is repeated until all nucleic acid probe species in the plurality of nucleic acid probe species fall between the minimum recovery rate threshold and the maximum recovery rate threshold.

Referring to FIG. 11, in some embodiments the disclosure provides a method 1100 for forming an adjusted nucleic acid probe set including a plurality of nucleic acid probe species. The method includes obtaining a first iteration of a nucleic acid probe set and splitting (1102) the probe set into a plurality of probe pools. The first iteration of the nucleic acid probe set includes the plurality of nucleic acid probe species distributed in a first plurality of pools. In some embodiments, the first plurality of pools is 3 pools. In some embodiments, the first plurality of pools is 2, 3, 4, 5, 6, 7, 8, 9, 10, or more pools.

The plurality of nucleic acid probe species includes, for each respective locus in at least a portion of a plurality of loci within a genome, a respective sub-plurality of nucleic acid probe species, where each respective nucleic acid probe species in the respective sub-plurality of nucleic acid probe species includes a different subsequence, or the complement of the different subsequence, of the respective locus that is not present in the plurality of loci other than the respective locus. For example, in some embodiments, the probe set tiles all or a portion of each of a plurality of genes.

Each respective nucleic acid probe species in the plurality of nucleic acid probe species is present in the first iteration of the nucleic acid probe set in a combination of a respective first proportion and second proportion that sums to a respective amount. Each nucleic acid probe species in the respective first proportion is a non-nucleotidic capture moiety conjugated version of the respective nucleic acid probe species and each nucleic acid probe species in the respective second proportion, when the respective second proportion is other than zero, is a capture moiety-free version of the respective nucleic acid probe species.

Each nucleic acid probe species present in a respective pool, in the first plurality of pools, aligns to a portion of the genome that is at least a minimum number of nucleotides away from any other portion of the genome that any other nucleic acid probe species present in a respective pool aligns with. In some embodiments, the minimum number of nucleotides is 50 nucleotides. In other embodiments, the minimum number is 25, 30, 35, 40, 45, 50, 55, 60, 65, 70, 75, 80, 85, 90, 95, 100, 125, 150, 200, 250, or more nucleotides. In this fashion the performance of each probe can be evaluated while minimizing the effects of adjacent probes that may overlap with the same target sequence.

Method 1100 also includes using 1104 each probe pool to capture target nucleic acids from a plurality of reference samples. In some embodiments, the plurality of reference samples is at least 2, 3, 4, 5, 6, 7, 8, 9, 10, 11, 12, 13, 14, 15, 16, 17, 18, 19, 20, 25, 30, 35, 40, 50, or more reference samples. In some embodiments, a relative amount of representation of each locus in the plurality of loci by the first plurality of reference nucleic acid samples is known. For example, in some embodiments, it is known that each locus in the plurality of loci is represented, in one or more of the reference samples, at a substantially similar amount. For example, if the reference samples are derived from genomic DNA known not to have copy number variations at the respective loci.

Method 1100 also includes determining (1106) a recovery rate for each probe. In some embodiments, a recovery rate is determined for each probe for each reference sample. In some embodiments, a measure of central tendency for the recovery of the probe is determined across all of the reference samples.

In some embodiments, method 1100 includes normalizing (1108) recovery rates, e.g., normalizing individual probe recovery rates by a measure of central tendency for all recovery rates for a particular reference sample. In some embodiments, method 1100 includes trimming (1110) high and/or low recovery rates for a probe across the reference samples. For example, in some embodiments, at least the high 10% of recovery rates across the plurality of reference samples are trimmed. In some embodiments, at least the high 5%, 10%, 15%, 20%, or 25% of recovery rates across the plurality of reference samples are trimmed. In some embodiments, at least the low 10% of recovery rates across the plurality of reference samples are trimmed. In some embodiments, at least the low 5%, 10%, 15%, 20%, or 25% of recovery rates across the plurality of reference samples are trimmed.

Accordingly, in some embodiments, the method includes separately analyzing each respective pool in the first plurality of pools in the first iteration of the nucleic acid probe set against a first plurality of reference nucleic acid samples to obtain a corresponding first recovery rate of each respective nucleic acid probe species in the plurality of nucleic acid probe species.

In some embodiments, method 1100 then includes adjusting (1112) conjugation proportions based on the recovery rates determined. For example, in some embodiments, the conjugation percentage of probes with a recovery rate above a maximum recovery threshold is lowered. In some embodiments, the conjugation percentage of probes with a recovery rate below a minimum recovery threshold is raised. In some embodiments, the conjugation percentage of probes with a recovery rate above a maximum recovery threshold is lowered and the conjugation percentage of probes with a recovery rate below a minimum recovery threshold is raised. In some embodiments, the adjustment is made proportional to a reference value. In some embodiments, the reference value is selected based upon the relative recovery rates across all of the probes, e.g., a recovery rate of a certain percentile of all recovery rates ranked. In some embodiments, the reference value is the recovery rate (e.g., a normalized recovery rate) at a percentile between the tenth percentile and the fiftieth percentile across the recovery rates for all probes in the pool. In some embodiments, reference value is the recovery rate (e.g., a normalized recovery rate) at a percentile between the tenth percentile and the fortieth percentile across the recovery rates for all probes in the pool. In some embodiments, the reference value is the recovery rate (e.g., a normalized recovery rate) at a percentile between the tenth percentile and the thirtieth percentile across the recovery rates for all probes in the pool. In some embodiments, reference value is the recovery rate (e.g., a normalized recovery rate) at a percentile at or about 10%, 11%, 12%, 13%, 14%, 15%, 20%, 25%, 30%, 35%, 40%, 45%, 50%, or an intermediate percentile thereof, across the recovery rates for all probes in the pool.

Accordingly, in some embodiments, the method includes identifying, based on the corresponding first recovery rate of each respective nucleic acid probe species, a first subset of the plurality of nucleic acid probe species that does not satisfy a maximum recovery rate threshold. And reducing, for each respective nucleic acid probe species in the first subset of the plurality of nucleic acid probe species, the respective first proportion while maintaining the requirement that the respective first proportion and second proportion for the respective nucleic acid probe species sums to the respective amount, to form a first adjusted version of the nucleic acid probe set.

In some embodiments, the respective first proportion is 100% in the first iteration of a nucleic acid probe set. Accordingly, in some embodiments, recovery rates falling below a minimum recovery threshold cannot be increased by increasing the proportion of the conjugated probe. However, in some embodiments, additional probe can be spiked-in in a subsequent iteration of the probe set to increase recovery for an under-performing probe.

In some embodiments, the plurality of loci is at least 100 loci. In some embodiments, the plurality of loci is at least 10, 25, 50, 75, 100, 125, 150, 175, 200, 250, 300, 400, 500, 750, 1000, 1250, 1500, 2000, 2500, 5000, 7500, 10,000, 15,000, 20,000, or more loci. In some embodiments, each locus in the plurality of loci is a gene.

In some embodiments, for the first iteration of the nucleic acid probe set, each nucleic acid probe species in the plurality of nucleic acid probe species is present in only one pool in the first plurality of pools.

In some embodiments, the first plurality of pools is three pools, and each respective pool in the first plurality of pools consists of, for a respective locus in the plurality of genomic loci, every third respective nucleic acid probe species in the respective sub-plurality of nucleic acid probe species for the respective locus, such that the first plurality of pools collectively consists of each respective nucleic acid probe species in the respective sub-plurality of nucleic acid probe species for the respective locus.

In some embodiments, in the first iteration of the probe set, the respective first proportion of each nucleic acid probe species in the plurality of nucleic acid probe species is the same. In some embodiments, in the first iteration of the probe set, the respective second proportion of each nucleic acid probe species in the plurality of nucleic acid probe species is zero.

In some embodiments, the amount of each nucleic acid probe species in the plurality of nucleic acid probe species is dimensioned as a specified molarity, and wherein the specified molarity is the same for each nucleic acid probe species in the plurality of nucleic acid probe species in the first iteration of the probe set. In some embodiments, the molarity of each nucleic acid probe species in the plurality of nucleic acid probe species is the same in a second iteration of the probe set that is based on the first adjusted version of the nucleic acid probe set.

In some embodiments, each nucleic acid probe species present in a respective pool, in the first plurality of pools, aligns to a portion of the genome that is at least 200 nucleotides away from any other portion of the genome to which any other nucleic acid probe species present in a respective pool aligns.

In some embodiments, the non-nucleotidic capture moiety is biotin, biotin carbonate 5, biotin carbamate 6, Iminobiotin, or Desthiobiotin. In some embodiments, the non-nucleotidic capture moiety is biotin.

In some embodiments, the different subsequence of the respective nucleic acid probe species in the respective sub-plurality of nucleic acid probe species is from 75 nucleotides to 250 nucleotides in length.

In some embodiments, the respective sub-plurality of nucleic acid probe species for a respective locus in the at least the portion of the plurality of loci consists of non-overlapping nucleic acid probe sequences.

In some embodiments, a gap between any two different subsequences within the respective locus, from any pair of respective nucleic acid probe species in the respective sub-plurality of probe species, is no more than 10 nucleotides. In some embodiments, a gap between any two different subsequences within the respective locus, from any pair of respective nucleic acid probe species in the respective sub-plurality of probe species, is no more than 1, 2, 3, 4, 5, 6, 7, 8, 9, 10, 11, 12, 13, 14, 15, 20, 25, 30, 35, 40, 45, 50, or more nucleotides.

In some embodiments, the sub-plurality of nucleic acid probe species for a respective locus in the plurality of loci consists of overlapping nucleic acid probe sequences.

In some embodiments, the plurality of nucleic acid probe species is at least 2000 nucleic acid probe species. In some embodiments, the plurality of nucleic acid probe species is at least 50, 100, 150, 200, 250, 300, 400, 500, 750, 1000, 1500, 2000, 2500, 300, 400, 500, 7500, 10,000, 12,500, 15,000, 20,000, 25,000, 30,000, 40,000, 50,000, or more nucleic acid probe species.

In some embodiments, the analyzing includes, for each respective pool in the first plurality of pools: capturing, for each respective reference nucleic acid sample in the plurality of reference samples, nucleic acids from the respective reference nucleic acid sample using the respective pool, measuring, for each respective nucleic acid probe species present in the respective pool, a respective first recovery rate for each respective reference nucleic acid sample in the plurality of nucleic acid reference samples, thereby obtaining a corresponding plurality of respective first recovery rates for the respective nucleic acid probe species, and determining, for each respective nucleic acid probe species present in the respective pool, the corresponding first recovery rate for the respective nucleic acid probe species based on the corresponding plurality of respective first recovery rates.

In some embodiments, the analyzing includes, for each respective pool in the first plurality of pools: capturing, for each respective reference nucleic acid sample in the plurality of reference samples, nucleic acids from the respective reference nucleic acid sample using the respective pool; amplifying, for each respective reference nucleic acid sample in the plurality of reference samples, the captured nucleic acids from the respective reference nucleic acid sample; measuring, for each respective nucleic acid probe species present in the respective pool, a respective first recovery rate for each respective reference nucleic acid sample in the plurality of nucleic acid reference samples based on the amplified nucleic acids, thereby obtaining a corresponding plurality of respective first recovery rates for the respective nucleic acid probe species, and determining, for each respective nucleic acid probe species present in the respective pool, the corresponding first recovery rate for the respective nucleic acid probe species based on the corresponding plurality of respective first recovery rates.

In some embodiments, the analyzing includes, for each respective pool in the first plurality of pools: amplifying nucleic acids in the respective pool, capturing, for each respective reference nucleic acid sample in the plurality of reference samples, amplified nucleic acids from the respective reference nucleic acid sample using the respective pool, measuring, for each respective nucleic acid probe species present in the respective pool, a respective first recovery rate for each respective reference nucleic acid sample in the plurality of nucleic acid reference samples based on the captured nucleic acids, thereby obtaining a corresponding plurality of respective first recovery rates for the respective nucleic acid probe species, and determining, for each respective nucleic acid probe species present in the respective pool, the corresponding first recovery rate for the respective nucleic acid probe species based on the corresponding plurality of respective first recovery rates.

In some embodiments, the analyzing includes, for each respective pool in the first plurality of pools: amplifying nucleic acids in the respective pool, capturing, for each respective reference nucleic acid sample in the plurality of reference samples, amplified nucleic acids from the respective reference nucleic acid sample using the respective pool, amplifying, for each respective reference nucleic acid sample in the plurality of reference samples, the captured nucleic acids from the respective reference nucleic acid sample, measuring, for each respective nucleic acid probe species present in the respective pool, a respective first recovery rate for each respective reference nucleic acid sample in the plurality of nucleic acid reference samples based on the amplified nucleic acids, thereby obtaining a corresponding plurality of respective first recovery rates for the respective nucleic acid probe species, and determining, for each respective nucleic acid probe species present in the respective pool, the corresponding first recovery rate for the respective nucleic acid probe species based on the corresponding plurality of respective first recovery rates.

In some embodiments, the first recovery rate of a respective nucleic acid probe species is determined by sequencing the captured or amplified nucleic acids and quantitating the number of raw sequence reads from the sequencing that overlap the respective nucleic acid probe by a minimum number of nucleic acids.

In some embodiments, the first recovery rate of a respective nucleic acid probe species is determined by sequencing the captured or amplified nucleic acids, de-duplicating raw sequence reads from the sequencing to generate unique sequence reads, and quantitating the number of unique sequence reads that overlap the respective nucleic acid probe by a minimum number of nucleic acids.

In some embodiments, the corresponding first recovery rate for the respective nucleic acid probe species is a measure of central tendency for some or all of the corresponding plurality of respective first recovery rates.

In some embodiments, the maximum recovery threshold is set relative to a measure of central tendency for the first recovery rate across all of the nucleic acid probe species.

In some embodiments, the maximum recovery threshold is no more than 10% greater than the measure of central tendency for the first recovery rate across all of the nucleic acid probe species.

In some embodiments, in the first adjusted version of the nucleic acid probe set: the respective first proportion of each respective nucleic acid probe species in the first subset of the plurality of nucleic acid probe species is at a lower proportion than the respective first proportion of each respective nucleic acid probe species in the plurality of nucleic acid probe species that satisfied the maximum recovery rate threshold.

In some embodiments, each respective first proportion of each respective nucleic acid probe species in the first subset of the plurality of nucleic acid probe species is reduced by an amount that is proportional to a difference between the corresponding first recovery rate for the respective nucleic acid probe and a maximum reference recovery rate. In some embodiments, the maximum reference recovery rate is the maximum recovery rate threshold. In some embodiments, the maximum reference recovery rate is set relative to the first recovery rate across all of the nucleic acid probe species present in the respective pool. In some embodiments, the maximum reference recovery rate is set to a value equal to a percentile first recovery rate across all of the nucleic acid probe species present in the respective pool. In some embodiments, the percentile recovery rate is a recovery rate between the tenth percentile and the fiftieth percentile for the first recovery rate across all of the nucleic acid probe species present in the respective pool.

In some embodiments, the method also includes identifying, based on the corresponding first recovery rate of each respective nucleic acid probe species, a second subset of the plurality of nucleic acid probe species that does not satisfy a minimum recovery rate threshold, and increasing, for each respective nucleic acid probe species in the second subset of the plurality of probe species, the respective first proportion while maintaining the requirement that the respective first proportion and second proportion for the respective nucleic acid probe species sums to the respective amount.

In some embodiments, the method also includes increasing, for each respective nucleic acid probe species in the second subset of the plurality of nucleic acid probe species, the respective first proportion proportional to a difference between the corresponding first recovery rate for the respective nucleic acid probe and a minimum reference recovery rate while maintaining the requirement that the respective first proportion and second proportion for the respective nucleic acid probe species sums to the respective amount.

In some embodiments, the method also includes obtaining a second iteration of the nucleic acid probe set, wherein the second iteration of the nucleic acid probe set comprises the plurality of nucleic acid probe species distributed in a second plurality of pools, where each respective nucleic acid probe species in the plurality of probe species is present in the second iteration of the nucleic acid probe set in a combination of a respective third proportion and fourth proportion that sums to a respective amount, wherein each nucleic acid probe species in the respective third proportion is a non-nucleotidic capture moiety conjugated version of the respective nucleic acid probe species and each nucleic acid probe species in the respective fourth proportion is a capture moiety-free version of the respective nucleic acid probe species, based on the first adjusted version of the final design for the nucleic acid probe set, and each nucleic acid probe species present in a respective pool, in the second plurality of pools, aligns to a portion of the genome that is at least 100 nucleotides away from any other portion of the genome that any other nucleic acid probe species present in a respective pool aligns with.

In some embodiments, the method also includes separately analyzing each respective pool in the second plurality of pools in the second iteration of the nucleic acid probe set against a second plurality of reference nucleic acid samples, in which a relative amount of representation of each locus in the plurality of loci by the second plurality of reference nucleic acid samples is known, to obtain a corresponding second recovery rate of each respective nucleic acid probe species in the plurality of nucleic acid probe species.

In some embodiments, the method also includes identifying, based on the corresponding second recovery rate of each respective nucleic acid probe species, a third subset of the plurality of nucleic acid probe species that does not satisfy a maximum recovery rate threshold.

In some embodiments, the method also includes reducing, for each respective nucleic acid probe species in the third subset of the plurality of nucleic acid probe species, the respective second proportion while maintaining the requirement that the respective first proportion and second proportion for the respective nucleic acid probe species sums to the respective amount to form a second adjusted version of the nucleic acid probe set.

In some embodiments, the method also includes identifying, based on the corresponding second recovery rate of each respective nucleic acid probe species, a fourth subset of the plurality of nucleic acid probe species that does not satisfy a minimum recovery rate threshold, and increasing each respective second proportion of each respective nucleic acid probe species in the fourth subset of the plurality of nucleic acid probe species.

In some embodiments, a first measure of a distribution of the second recovery rates across all of the nucleic acid probe species in the second iteration of the nucleic acid probe set is at least 25% smaller than a second measure of a distribution of the first recovery rates across all of the nucleic acid probe species in the first iteration of the nucleic acid probe set.

In some embodiments, the analyzing includes, for each respective pool in the second plurality of pools: capturing, for each respective reference nucleic acid sample in the plurality of reference samples, nucleic acids from the respective reference nucleic acid sample using the respective pool, measuring, for each respective nucleic acid probe species present in the respective pool, a respective second recovery rate for each respective reference nucleic acid sample in the plurality of nucleic acid reference samples, thereby obtaining a corresponding plurality of respective second recovery rates for the respective nucleic acid probe species, and determining, for each respective nucleic acid probe species present in the respective pool, the corresponding second recovery rate for the respective nucleic acid probe species based on the corresponding plurality of respective second recovery rates.

In some embodiments, the analyzing includes, for each respective pool in the second plurality of pools: capturing, for each respective reference nucleic acid sample in the plurality of reference samples, nucleic acids from the respective reference nucleic acid sample using the respective pool, amplifying, for each respective reference nucleic acid sample in the plurality of reference samples, the captured nucleic acids from the respective reference nucleic acid sample, measuring, for each respective nucleic acid probe species present in the respective pool, a respective second recovery rate for each respective reference nucleic acid sample in the plurality of nucleic acid reference sample based on the amplified nucleic acids, thereby obtaining a corresponding plurality of respective second recovery rates for the respective nucleic acid probe species, and determining, for each respective nucleic acid probe species present in the respective pool, the corresponding second recovery rate for the respective nucleic acid probe species based on the corresponding plurality of respective second recovery rates.

In some embodiments, the analyzing includes, for each respective pool in the second plurality of pools: amplifying nucleic acids in the respective pool, capturing, for each respective reference nucleic acid sample in the plurality of reference samples, amplified nucleic acids from the respective reference nucleic acid sample using the respective pool, measuring, for each respective nucleic acid probe species present in the respective pool, a respective second recovery rate for each respective reference nucleic acid sample in the plurality of nucleic acid reference sample based on the captured nucleic acids, thereby obtaining a corresponding plurality of respective second recovery rates for the respective nucleic acid probe species, and determining, for each respective nucleic acid probe species present in the respective pool, the corresponding second recovery rate for the respective nucleic acid probe species based on the corresponding plurality of respective second recovery rates.

In some embodiments, the analyzing includes, for each respective pool in the second plurality of pools: amplifying nucleic acids in the respective pool, capturing, for each respective reference nucleic acid sample in the plurality of reference samples, amplified nucleic acids from the respective reference nucleic acid sample using the respective pool, amplifying, for each respective reference nucleic acid sample in the plurality of reference samples, the captured nucleic acids from the respective reference nucleic acid sample, measuring, for each respective nucleic acid probe species present in the respective pool, a respective second recovery rate for each respective reference nucleic acid sample in the plurality of nucleic acid reference sample based on the amplified nucleic acids, thereby obtaining a corresponding plurality of respective second recovery rates for the respective nucleic acid probe species, and determining, for each respective nucleic acid probe species present in the respective pool, the corresponding second recovery rate for the respective nucleic acid probe species based on the corresponding plurality of respective second recovery rates.

Another aspect of the present disclosure provides a balanced nucleic acid probe set prepared according to any one of the methods and/or embodiments disclosed herein.

Yet another aspect of the present disclosure provides a computer system having one or more processors, and memory storing one or more programs for execution by the one or more processors, the one or more programs comprising instructions for performing any of the methods and/or embodiments disclosed herein. In some embodiments, any of the presently disclosed methods and/or embodiments are performed at a computer system having one or more processors, and memory storing one or more programs for execution by the one or more processors. Another aspect of the present disclosure provides a non-transitory computer readable storage medium storing one or more programs configured for execution by a computer, the one or more programs comprising instructions for carrying out any of the methods disclosed herein.

### EXAMPLES

### Example 1—Probe Set Design

BRCA1 and BRCA2 are genes that are known to have a prevalence of large INDEL (insertion/deletion) variants that are clinically relevant. For example, the presence of an INDEL variant in the BRCA1 or BRCA2 gene in a germline/non-cancerous specimen from a patient may be associated with a particular risk for developing breast cancer. For example, the presence of an INDEL variant in the BRCA1 or BRCA2 gene in a somatic/cancer specimen from a patient may be associated with a particular prognosis, diagnosis, and/or matching therapy likely to be effective in slowing the progression of the patient's cancer.

However, large INDELs can be difficult to detect by next generation sequencing (NGS) because of the nature of short read sequencing by synthesis NGS technology. In this example, the systems and methods may be used to more uniformly sequence a BRCA gene (for example, resulting in similar numbers of sequencing reads associated with each region targeted by a probe during hybridization capture), which may facilitate the detection of INDEL variants in the BRCA gene.

In this example, the systems and methods receive a genetic sequence associated with a human BRCA gene. The sequence may be received from a database such as the National Center for Biotechnology Information (NCBI) or a similar database of genetic sequences. This example may apply to the BRCA 1 and/or BRCA 2 gene.

The genetic sequence may be annotated or the systems and methods may annotate the genetic sequence. Annotation may include labeling portions of the genetic sequence as a start sequence, promoter region(s), another class of genetic region, etc.

The systems and methods may design a plurality of probes or receive a set of BRCA1 or BRCA2 probes (for example, probes for hybridization capture, for example, for use during library generation for next generation sequencing) and each probe may target a distinct genetic locus associated with the BRCA1 or BRCA2 gene. The regions targeted by probes may be spaced uniformly across the BRCA1 or BRCA2 gene (for example, having approximately the same number of bases between each target), or the regions targeted by probes may be concentrated in certain regions of the BRCA1 or BRCA2 gene. As an example, a high density of probes designed toward a target region could be needed due to a high prevalence of known recurring genetic mutations in that region (for example, the region may be a hotspot). In another example, a high density of probes designed toward a target region could be needed due to unfavorable hybridization kinetics or specificity of probes that target that region. Target regions may all be located in exon regions, intron regions, promoter regions, or any combination thereof. It is also possible to include regularly spaced probes at any spacing (for example, 1 probe per 10 kB, 100 KB, 1 MB, etc.), which may be done throughout an entire genome or a portion of the genome. In one example, each probe is 120 base pairs long.

Probes covering the BRCA genes could be designed as one probe per exon or multiple probes per exon that could be tiled end-to-end (for example, the nucleotide targeted by the end of one probe is adjacent to a nucleotide targeted by the neighboring probe, but there are no nucleotides targeted by both a probe and a neighboring probe), overlap (for example, one or more adjacent nucleotides may be targeted by more than one probe), or spaced apart (for example, there may be untargeted nucleotides between the nucleotides targeted by a first probe and the nucleotides targeted by a second probe). Probes covering the BRCA genes could also include probes targeting the intronic regions. Intronic probes could include a single probe per intron or multiple probes per intron that are regularly or irregularly spaced. Probes covering the BRCA genes could also include probes targeting the promoter regions of the genes with one or multiple probes. Probes could also be designed and included to target intergenic regions neighboring the BRCA genes with one or multiple probes.

Probe design may be fully manual, or partially or entirely automated through the use of a probe design software program.

The plurality of probes may be used during the generation of a sequencing library (for example, for enrichment for next generation sequencing) from one or more test specimens or control samples known to comprise a BRCA region in order to confirm that the plurality of probes align to the relevant target regions in the BRCA gene. In another embodiment, testing may be accomplished using in silico methods, which may include the use of probe design software.

The probe design may account for unique aspects of the BRCA gene. For instance, certain regions of the BRCA gene are expected to contain large deletions and/or duplications (for example, INDEL variants) that span a portion of an exon or an intron, are approximately 1 kilobase or larger in size, span one or more exons and/or introns, or may be of varying sizes (for example, INDELs caused by alu insertions). For an example of BRCA1 or BRCA2 INDELs, see Schmidt A Y et al, J Mol Diagn., 19(6):809-16 (2017), the contents of which are incorporated by reference herein in their entirety. As another example, probes may be designed to provide coverage across exonic regions of the BRCA gene, intronic regions of the BRCA gene, or both exon and intron regions of the BRCA gene.

The probes may be tested and adjusted to achieve even sequencing coverage across the entire BRCA1 or BRCA2 gene, including promoter(s), exons, and introns (for example, each probe may be adjusted such that next generation sequencing results in approximately the same number of sequencing reads mapping to each region targeted by a probe).

In order to compare the number of reads associated with each target region, a sequencing library may be prepared from one or more test specimens or control samples known to comprise a wildtype or normal BRCA gene, using the plurality of probes. In one example, the test specimen is a solid specimen (for example, a tumor biopsy, an FFPE tissue section, etc.). In another example, the test specimen is a liquid specimen (for example, a blood specimen, a liquid biopsy specimen, etc.).

For each probe, multiple copies (molecules) of the probe may be used for hybridization and capture during library generation. Each individual probe molecule may or may not be biotinylated or labeled by another labeling molecule. For each probe, the proportion or percentage of individual probe molecules that are labeled (for example, biotinylated) may be known and adjusted. The concentration of each probe may be measured (for example, in molarity units, or other similar units used for measuring the concentration of a molecule). In one example, each probe is added in an amount of approximately 0.1 to 100 picomolar (pM). For each probe, the concentration may be adjusted.

For each probe, the systems and methods may adjust the percentage of the individual probe molecules that are biotinylated, for example, based on the coverage calculated for each probe (for example, the number of reads associated with each target region). For instance, the biotinylation percentage of each probe that targets the BRCA gene may be adjusted depending on the number of reads from that probe in comparison to reads of other probes targeting other loci in the BRCA gene. As another example, the biotinylation percentage of each probe in the plurality of probes may be adjusted depending on the number of reads from that probe in comparison to reads of other regions in the BRCA gene. In some embodiments, more than one probe may be responsible for producing reads for a region.

Row 1 in Table 1 shows the number of reads associated with each of five hypothetical probes targeting the BRCA gene, where each probe is 100% biotinylated. The third row shows the new biotinylation percentages (33.2, 91.1, 26.9, 34.4, and 56.5%), selected based on the number of reads associated with each probe. In this example, the new biotinylation percentages should result in each probe being associated with approximately 71 reads (approximately the same percentage of the total reads). Other biotinylation percentages could be selected such that each probe is associated with an approximately equal number of reads. For example, 16.6, 45.5, 13.4, 17.2, and 28.2% may result in each probe being associated with approximately 71 reads.

The biotinylation percentages may be adjusted for each probe and tested to determine the number of reads associated with each probe at the new biotinylation percentage. If the number of reads associated with each probe is highly variable, the biotinylation percentage may be adjusted again. These steps may be repeated multiple times, for example, until the number of reads associated with each probe is less variable.

Five probes are shown in this example, but in reality 1,000, 10,000, 100,000 or more probes may be used to cover the BRCA gene and a new biotinylation percentage may be calculated and tested for each probe.

In various embodiments, having an approximately equal number of reads associated with each target region may facilitate the detection of duplications and/or deletions (INDELs) in a BRCA gene, for example, in a specimen having a BRCA gene that has deletions, duplications, or is otherwise not wildtype or not normal.

TABLE 1

| | | | | | | Combined |
|---|---|---|---|---|---|---|
| | | | | | | Total |
| | Probe 1 | Probe 2 | Probe 3 | Probe 4 | Probe 5 | Reads |
| No. of reads | 85 | 31 | 105 | 82 | 50 | 353 |
| Percent of | 24.1% | 8.8% | 29.7% | 23.2% | 14.2% | 100.0% |
| total Reads | | | | | | |
| New | 0.332 | 0.911 | 0.269 | 0.344 | 0.565 | |
| biotinylated | | | | | | |
| New Percent | 20.0% | 20.0% | 20.0% | 20.0% | 20.0% | |
| activity | | | | | | |
| Predicted | 71 | 71 | 71 | 71 | 71 | 353 |
| total reads | | | | | | |

After the biotinylation percentage is adjusted for each probe, such that the number of reads associated with each probe is approximately equal, the systems and methods may compare the number of reads associated with the entire BRCA gene to the number of reads associated with each of a plurality of additional genes selected from a targeted sequencing panel.

The systems and methods may adjust the concentration of the BRCA gene probes based on the number of reads associated with each selected gene in the sequencing panel. The concentration of the BRCA gene probes may be adjusted in an attempt to have approximately the same number of BRCA gene reads as the number of reads associated with each gene selected for the comparison. Methods other than concentration adjustment may be used. For example, the proportion of probe molecules having capture moieties may be reduced for all probes targeting genes associated with a large number of reads.

Table 2 illustrates the concept of altering the concentration of a probe or pool of multiple probes to attempt to achieve more uniform coverage. Historically, adjusting the concentrations of probes does not always result in a predictable change in hybridization kinetics and may have other off target effects.

Table 2 shows the number of reads associated with each of four hypothetical genes and the BRCA gene, where the plurality of probes associated with each gene have a measurable concentration. The third row shows the factor by which the original concentration may be multiplied to generate a new concentration such that all genes have approximately 730 reads. Other concentration adjustment factor values could be selected such that each gene is associated with an approximately equal number of reads. For example, 0.930×, 1.916×, 0.606×, 0.881×, and 1.622× may result in each gene being associated with approximately 730 reads.

Five genes are shown in this example, but in reality 20,000 genes, or hundreds of thousands of alleles or transcripts of genes may be included in a targeted sequencing panel and a concentration adjustment factor may be calculated for each one.

The concentrations may be adjusted for each probe set (for each gene) and tested to determine the number of reads associated with each gene at the new concentration. If the number of reads associated with each gene is highly variable, the concentration may be adjusted again. These steps may be repeated multiple times, for example, until the number of reads associated with each gene is less variable.

Additional concentration adjustments may include: increasing the concentration(s) of vastly underperforming probe(s) (for example, probes that are associated with a lower number of reads), and/or establishing multi-tiered coverages (for example, instead of the entire genome having a uniform coverage, a first region of the genome may have a first coverage, a second region of the genome may have a second coverage, a third region of the genome may have a third coverage, etc.). To illustrate an example of multi-tiered coverage, the BRCA1 or BRCA2 gene may have a coverage of 500× and the rest of the panel may have a coverage of 150×. In alternative embodiments, any gene of interest or gene that is difficult to sequence may have a coverage that is higher than other portions of a genome. For example, a gene of interest may have a coverage of 10,000×.

TABLE 2

| | BRCA gene | Gene 2 | Gene 3 | Gene 4 | Gene 5 |
|---|---|---|---|---|---|
| No. of reads | 785 | 381 | 1205 | 829 | 450 |
| Concentration | 0.465 | 0.958 | 0.302 | 0.440 | 0.811 |
| adjustment | | | | | |
| factor | | | | | |

The systems and methods may report any detected INDEL variants in the BRCA1 or BRCA2 gene of a patient specimen to a geneticist or medical professional in order to aid the professional in counseling or treating the patient.

The systems and methods may apply the concepts of biotinylation percentage adjustments, concentration adjustments, and other adjustments to affect probe performance (for example, to achieve uniform coverage across a genetic region) in additional use cases other than detecting INDEL variants in the BRCA1 or BRCA2 gene. In one example, the systems and methods are used to generate more uniform coverage of a TP53 gene.

For example, the systems and methods may be used to achieve uniform coverage of a CYP gene (for example, CYP2D6) to facilitate the detection of reads from CYP pseudogenes, rearrangements, INDEL variants and/or copy number variants (CNVs) in the CYP gene. If the systems and methods detect CYP gene variants or other CYP gene-related data in a patient specimen, the systems and methods may inform a physician, medical professional, or geneticist about the variant or data and any known or predicted effects that the variant(s) or data may have on the patient's RNA expression levels (for example, for a CYP gene, for each allele of a CYP gene) and/or drug metabolism rate.

The systems and methods may be used to facilitate determining if a sequencing read is associated with a pseudogene to prevent inaccurately aligning a pseudogene read to a gene having a sequence that is similar to the pseudogene.

In one example, the systems and methods may optimize a probe set to achieve more even coverage of the PMS2 gene and/or PMSCL pseudogene to facilitate the accurate alignment of sequencing reads to either the PMS2 gene or the PMSCL pseudogene. Certain, known variants in the PMS2 gene are associated with an increased risk for multiple cancer types (for example, colorectal, endometrial, ovarian, stomach, urinary cancer, etc.) If a PMS2 variant is detected by NGS without the use of an optimized probe set, it may be difficult to be certain that the variant is in PMS2 and not in the PMSCL pseudogene. If the systems and methods detect a PMS2 variant associated with increased risk for developing cancer, the systems and methods may inform a patient, physician, medical professional, or geneticist of the presence of the PMS2 variant in the patient.

In one example, the systems and methods may optimize a probe set to facilitate the detection of exon skipping, splice variants, alternative splicing, or differential splicing of a gene with the use of NGS or RNA-seq. In various embodiments, splice variants could be generated by fusion events, splice sites, mutations in genes encoding for splice factors, etc. Exon skipping may be difficult to detect by DNA-seq.

In one example, the systems and methods may optimize a probe set to generate more uniform coverage of the DMD gene. The DMD gene is very large (at least 2,300 kb long) and has approximately 80 exons. There are many splice variants (for example, skipped exons) for this gene, and more uniform coverage of the gene transcripts would facilitate detection of splice variants. In some examples, the splice variant is an inherited germline variant. In some examples, exon skipping in the DMD gene has clinical relevance for Duchenne muscular dystrophy. For example, exon skipping in an mRNA transcript of the DMD gene may prevent ribosomes from translating the DMD mRNA into dystrophin protein, exacerbating the muscular dystrophy. For patients with a particular skipped exon, a treatment (for example, eteplirsen) may be recommended to induce production of dystrophin protein (often a shortened version of the protein) from DMD mRNA missing certain exons. The systems and methods may report detected DMD variants and any prognosis, diagnosis, and/or matched therapy associated with the detected variants.

In another example, the systems and methods optimize probes for detecting exon skipping in the MET gene (for example, MET Exon 14 skipping). In various embodiments, if exon 14 of MET gene is spliced (skipped), the cell is more active or oncogenic. For example, cancer cells may have exon 14 skipped as a somatic mutation, especially non-small cell lung cancer (NSCLC). Patients having a MET splice variant may respond favorably (for example, showing a slowed progression of cancer or disease) to treatment with MET targeted therapies (for example, capmatinib, crizotinib, pembrolizumab, MET tyrosine kinase inhibitors, etc.) For an example of therapies and trials targeting MET splice variants, see Reungwetwattanaa T. et al, Lung Cancer, 103:27-37 (27), the content of which is incorporated herein in its entirety for all purposes. The systems and methods may report any detected MET splice variants and any prognosis, diagnosis, and/or matched therapy associated with the detected MET splice variants.

In one example, the systems and methods may optimize a probe set to facilitate the detection of fusions (for example, RNA fusions) with the use of NGS.

In one example, the ALK gene can form fusions with a variety of partner genes, especially in cancer cells (for example, NSCLC). This variety of genes that can partner with ALK gives rise to a variety of fusion variants, many of which have not been previously characterized by scientific research publications. One example of a partner gene is EML4. In an EML4-ALK fusion, EML4 expression is driving the ALK expression. (In various embodiments, any gene could be the partner gene).

The fusion variant could cause differential expression on the 3′ side of the ALK gene vs the 5′ side of the ALK gene. For example, the systems and methods may facilitate the detection of non-equal expression levels of ALK exon 1 and the final exon of ALK. This information may indicate the presence of a fusion variant. The systems and methods may also improve the uniformity of coverage at each exon of ALK, to facilitate locating which exon in ALK contains the fusion breakpoint.

In various examples, exon 20 in the ALK gene is a common breakpoint. If a fusion formed with the 3′ side of a partner gene and the 5′ side of the ALK gene (starting in exon 20 of the ALK gene), then exon 20 and beyond of the ALK gene would be upregulated (for example, have higher expression levels than the exons on the 3′ side of the breakpoint). In another example, a different ALK gene exon could serve as a breakpoint and then all exons on the 5′ side of the breakpoint would be upregulated. The systems and methods may be used to facilitate the detection of upregulation (increased expression level) or downregulation (reduced expression level) of some exons in ALK compared to other ALK exons in order to detect an ALK fusion and/or the location of the fusion breakpoint in the ALK gene. In one example, sequencing data from as few as one cDNA fragment may be used to detect a fusion variant and/or determine breakpoints for a fusion variant.

In various embodiments, the systems and methods optimize a probe set to provide more uniform coverage of each exon of the ALK gene to improve the signal to noise ratio such that the data may be used to generate more refined and accurate exon-level expression calls, or expression levels for the individual exons (for example, increasing the RNA expression level resolution to the scale of individual exons).

In various embodiments, in a somatic (for example, cancer or tumor) specimen, if the tumor purity is low (for example, only 10% or so), more uniform coverage/sensitivity is even more important for accurately analyzing sequencing data to detect variants.

In some embodiments, probes are targeted for sequencing antimicrobial resistance genes (AMR), antiviral drug resistance genes, or the genes targeted by antimicrobial therapeutics. Probes may consist of optimized probe sets for rare or novel drug resistance genes. Probes may consist of panels for specific coinfections, groups of related infectious agents, which may be grouped according to one or more of the following criteria: the infectious agents cause similar symptoms, affect similar geographical locations and/or anatomical areas, or have similar organism phylogeny. Application examples might include selection of cfDNA for detecting variation in drug resistant Borrelia burgdorferi, the causative agent for Lyme Disease (for an example of varying degrees of AMR in Borrelia burgdorferi, see Hodzic E, Bosnian Journal of Basic Medical Sciences, 7 Jul. 2015, 15(3):1-13 DOI: 10.17305/bjbms.2015.594 PMID: 26295288 PMCID: PMC4594320, the contents of which are incorporated herein by reference in their entirety). Typically Lyme disease can be an initial mild infection in the body and can be dormant and reactivated, causing unusual symptoms. The systems and methods could be applied to sequencing “persister” cases where infectious agents have a dormant metabolism, for example, as in the case of Lyme disease. In this example, the systems and methods may be used to 1) to increase the selection of genomic DNA or RNA transcripts from the target organism (for example, an infectious agent), 2) to selectively remove nucleic acids having a high copy number, high number of RNA transcripts or redundant DNA fragments from the sequencing library preparation 3) Select transcripts involved in host response to a pathogenic infection (IGF1, TLR9 gene in reference to Meningitis, host immune genes, etc.). For an example of the interaction between host immune response genes and pathogenicity of an infectious agent, see Sanders M S et al., Genes Immun. 2011 July; 12(5):321-34 (2011), the contents of which are incorporated herein by reference in their entirety for all purposes. In various embodiments, the combined effect of the sequencing library preparation strategies included in the systems and methods may allow for an increase in the ratio of desired targets of rare populations of RNA transcripts or DNA molecules in the subsequent sequencing reactions above the background level to increase detection of and the ability to call rare variants or coinfections. For an example of using NGS hybrid-capture in infectious diseases, see Gaudin and Desnues, Front Microbiol., 9:2924 (2018), the contents of which are incorporated herein in their entirety for all purposes. These could be used in the calling of viral or bacterial origin of infection (for example, detecting the presence of an infectious agent in a patient specimen and/or determining which infectious agent is the cause of a disease if multiple infectious agents are present). For an example of using host RNA expression levels to determine which infectious agent is responsible for disease, see Herberg J A et al., JAMA, 316(8):35-845 (2016), the contents of which are incorporated herein in their entirety for all purposes. The tuning of the AMR probe set to enrich rare sequence information may allow health system wide information (for example, data generated by multiple medical treatment centers and stored in a database) about AMR tracking as well. For an example of AMR tracking, including determining the genetic sequences in infectious agents associated with AMR, see Guitor et al., Antimicrob Agents Chemother, 64(1):e01324-19 (2019), the contents of which are incorporated herein in their entirety for all purposes. Additionally, capture probes can be developed for accessing host response to infection where limiting the conversion of highly expressed genes would allow for the selective capture of rare target or splice variants of RNA transcripts to be accessed to determine the host response to the causative agent, the location of the infection, or early indications of organ rejection due to infection.

In some embodiments, probes may be designed for panels of coinfections, panels of widely divergent organisms across many genera of distantly and/or closely related organisms. These may be used in immunocompromised individuals who could have an opportunistic infection with an organism that is rarely pathogenic. The panel could be comprised of probes for infectious agents for a genus that is unknown to be a pathogen, normal flora, or an emerging infectious disease. These organisms may be unculturable, and therefore would remain undetected with standard of care procedures. In one embodiment, an ideal application of co-infection panels would be for immunocompromised patients who may have several active infections, for example HIV positive patients with pneumonia could quickly be prescribed a course of therapy based on determination if the pneumonia is caused by methicillin resistant Staphylococcus aureus, multidrug-resistant Streptococcus pneumoniae, ciprofloxacin resistant Pseudomonas aeruginosa, or another microbe. In another example, patients who have received an organ transplant and are on drugs suppressing their immune system may benefit from a wide panel of probes targeting genes whose expression levels can be indicative of organ failure, each of which may be adjusted according to the systems and methods disclosed herein.

In various embodiments, the systems and methods optimize probe sets to achieve more uniform coverage of the fms-related tyrosine kinase 3 (FLT3) gene to facilitate detection of tandem repeats/duplications by NGS. Certain FLT3 tandem repeats may be associated with a prognosis, diagnosis, or matched therapy (for example, in an acute myeloid leukemia cancer specimen). For example, see Spencer D H et al., J Mol Diagn., 15(1):81-93 (2013), the contents of which are incorporated herein by reference in their entirety for any and all purposes. The systems and methods may report detected FLT3 tandem repeats and any associated prognosis, diagnosis, and/or matched therapies predicted to be effective in slowing the progression of AML or another disease.

In various embodiments, the systems and methods optimize probe sets to achieve more uniform coverage of t-cell receptor or b-cell receptor (TCR/BCR) genes to give more accurate clonal population statistics, which may be used to characterize an immune repertoire; to monitor immune response, autoimmune disease, cancer progression, minimal residual disease (MRD), immunotherapy treatment; to design novel immunotherapies; or to predict susceptibility to various infectious diseases.

In various embodiments, the systems and methods may be used to make probes multi-use, achieving similar sensitivity of targets across various applications (e.g. solid tumor versus liquid biopsy, or targeted panel versus whole exome or whole genome), which may include adjusting a probe's ratio of capture moiety-conjugated probes for each panel.

In various embodiments, the systems and methods may include a 3 tool process that is run in series, wherein the three tools are: 1. Modify the activity of each probe without affecting the stoichiometry. Modifying the percent of probe biotinylation without changing the total molarity may allow for very accurate fine tuning of the activity. 2. Adjusting the relative probe concentrations to alter the relative recovered target through stoichiometry. 3. Adding more probes to the region (or probe design change).

In various embodiments, the systems and methods may be used in conjunction with sequencing DNA from solid, blood, liquid biopsy, or other specimens, or RNA. In various embodiments, the systems and methods may facilitate the more accurate detection of single nucleotide variants (SNVs), small INDELs, large INDELs, CNVs, pseudogenes, GC/AT rich regions of the genome, genetic rearrangements, splice variants, gene expression levels, aneuploidy, trisomy, and other possible conclusions based on genetic sequencing results. In various embodiments, the systems and methods may facilitate genetic analysis of genetic regions of interest of varying sizes, including point locations, small regions or elements, individual exon or intron, multiple exons or multiple introns, entire gene, partial chromosome, whole chromosome, etc. In various embodiments, the systems and methods may be utilized for genetic sequencing in the following categories: oncology/somatic, germline, infectious or parasitic disease, microbiome, other areas of human healthcare, etc.

The methods and systems described above may be utilized in combination with or as part of a digital and laboratory health care platform that is generally targeted to medical care and research. It should be understood that many uses of the methods and systems described above, in combination with such a platform, are possible. One example of such a platform is described in U.S. patent application Ser. No. 16/657,804, titled “Data Based Cancer Research and Treatment Systems and Methods”, and filed Oct. 18, 2019, which is incorporated herein by reference and in its entirety for all purposes.

### Example 2—Balancing Probe Sets

FIGS. 8A-8D illustrate an example method of balancing probe sets, in accordance with some embodiment of the present disclosure.

A schematic of a method for obtaining a first iteration of a nucleic acid probe set for a plurality of genomic loci is illustrated in FIG. 8A, comprising a plurality of nucleic acid probe species distributed in a first plurality of pools. A full probe set comprising a plurality of nucleic acid probe species 802 (top panel; 802-a, 802-b, 802-c, 802-d, 802-e, 802-f, 802-g, 802-h, 802-i) was obtained. Each nucleic acid probe species in the probe set included a nucleic acid sequence that was aligned to a portion of a genome, as illustrated by overlapping nucleic acid fragments 804 (e.g., 804-a, 804-b, 804-d, 804-f, 804-h). Notably, some fragments were targeted for enrichment by only one probe, while other fragments were targeted by a plurality of probes. For instance, fragment 804-a had partial complementarity to probe 802-a alone, while fragment 804-b had partial complementarity to both probe 802-a and probe 802-b. Fragments 804-d, 804-f, and 804-h were similarly complementary to a plurality of probes. The inclusion of multiple neighboring probes, each having complementarity to overlapping portions of a given genomic locus, can result in uneven coverage during enrichment and sequencing analysis (e.g., next-generation sequencing analysis).

The lower panels of FIG. 8A illustrate a method for balancing probe sets by dividing the plurality of nucleic acid probe species into a plurality of pools, thus reducing overlapping and/or neighboring probe effects that can result in uneven coverage during analysis. For instance, the plurality of nucleic acid probe species in the probe set was divided into three pools. Each pool included a subset of nucleic acid probe species, where each respective nucleic acid probe species had sequence complementarity to a respective genomic locus but did not overlap with any other genomic locus to which another nucleic acid probe species in the respective subset aligned.

Thus, for example, the first pool included a first subset of nucleic acid probe species including probes 802-a, 802-d, and 802-g. Accordingly, probe 802-a had at least partial complementarity to a first sub-plurality of overlapping nucleic acid fragments including fragments 804-a and 804-d. Probe 802-d had at least partial complementarity to a second sub-plurality of overlapping nucleic acid fragments including probe 804-h, and probe 802-g had at least partial complementarity to a third sub-plurality of overlapping nucleic acid fragments. As highlighted by fragments 804-d and 804-h, each sub-plurality of nucleic acid fragments targeted by each respective nucleic acid probe species in the first subset did not overlap with any other sub-plurality of nucleic acid fragments. In other words, each genomic locus represented by the subset of probes in the first pool was targeted by no more than one probe.

The second pool (including probes 802-b, 802-e, and 802-h) and the third pool (including probes 802-c, 802-f, and 802-i) were similarly divided such that each sub-plurality of nucleic acid fragments targeted by each respective nucleic acid probe species in each respective subset did not overlap.

FIG. 8B illustrates a plurality of recovery rates for the plurality of nucleic acid probe species in the example nucleic acid probe set. The recovery rates for each respective nucleic acid probe species were determined using the count of sequence reads, obtained from a sequencing reaction, overlapping the respective nucleic acid probe species (e.g., coverage). The mean recovery rate (mean coverage) was calculated across the plurality of nucleic acid probe species in the nucleic acid probe set and used to normalize the coverage for each respective nucleic acid probe species. The relative probe coverage normalized to the mean coverage was then plotted for each probe species in the probe set. A wide range of coverage was observed across the plurality of probes species in the probe set. For instance, probe 802-a exhibited relatively high coverage at approximately 1.5, probe 802-c exhibited relatively low coverage at approximately 0.5, and probe 802-b exhibited coverage at or near the mean.

A correction value was determined in order to adjust the level of probe detection for probes with coverage that deviated from the mean. For each probe species in the probe set, the coverage percent of mean was determined (e.g., a measure of the difference between the corresponding recovery rate of the respective nucleic acid probe species and the measure of central tendency for the recovery rate of all of the nucleic acid probe species in the probe set). Using the coverage percent of mean, a relative correction to the mean was determined, indicating an appropriate level of adjustment for each respective probe. As FIG. 8C illustrates, probe 802-a exhibited 156.3% coverage compared to the mean, confirming earlier results observed in FIG. 8B and warranting a correction of −37%. Conversely, probe 802-c exhibited only 49.9% coverage compared to the mean, which could be corrected by increasing probe detection by 97%. Probe 802-b exhibited near-mean coverage at 100.3%, resulting in a correction value of only −2%.

FIG. 8D illustrates another schematic showing how adjustment of relative probe detection by increasing or decreasing the proportions of capture moieties (e.g., biotin) for respective probe species can be used to balance the results of sequencing analysis (e.g., coverage). In some instances, a plurality of nucleic acid probe species in a probe set can comprise probe species that perform at varying efficiencies and cause uneven coverage. Low performing probes may be poorly detected, resulting in underrepresentation at corresponding genomic loci (e.g., valleys), whereas high performing probes may be detected at levels well above the mean, resulting in overrepresentation (e.g., peaks). By increasing the proportions of conjugated to unconjugated probes for low performing probe species, detection of these probes can be increased. Similarly, by decreasing the proportions of conjugated to unconjugated probes for high performing probe species, detection of these probes can be decreased. Adjustment of the proportions of capture moiety conjugated (e.g., biotinylated) probes across the plurality of nucleic acid probe species for a nucleic acid probe set can allow for balanced and even coverage during, for example, sequencing analysis.

### Example 3—Balancing Probe Sets

To evaluate several variations of the probe balancing methodologies described herein, a probe set tiling 105 genes was divided into three pools by selecting every third probe. That is, the first pool included every third probe starting from probe 1, the second pool included every third probe starting with probe 2, and the third pool included every third probe starting with probe 3. Each probe in the probe set was approximately 120 nucleotides long and the probes did not overlap, meaning that the target sequence for each probe in each of the pools was separated by at least 240 nucleotides. The first pool, containing 996 probe species each 100% biotinylated, was then used to enrich for nucleic acids in twenty DNA libraries containing genomic DNA fragments that were each prepared from a different genomic samples. The average size of the genomic DNA fragments was less than 240 nucleotides, such that the majority of DNA molecules in each library were targeted by no more than one probe in the pool of probes.

The enriched nucleic acids were sequenced and sequence reads, either raw sequence reads or deduplicated sequences determined therefrom, were mapped to each probe. Recovery was then calculated for each probe in each sample based on raw (pre-deduplicated) sequence reads and deduplicated sequence reads. Example data for pre-deduplicated recovery and post-deduplicated recovery data from several probes targeting the ERRFII gene are shown in Tables 3 and 4, respectively. The recovery data for each respective sample of the twenty samples was then normalized by dividing the coverage of each probe (either pre-deduplicated or post-deduplicated) by the average coverage for the respective sample. The normalized coverage for each probe was then ranked among the twenty samples, and the normalized data was trimmed by removing the highest four normalized values and the lowest four normalized value for each probe. Statistics for the trimmed and normalized recovery for the 5 example probes are shown in Tables 3 and 4, respectively.

TABLE 3
Recovery data for example probes in the first pool using pre-deduplicated coverages.

| | Probe 1 | Probe 2 | Probe 3 | Probe 4 | Probe 5 |
|---|---|---|---|---|---|
| Probe start position | 8073263 | 8073623 | 8073983 | 8074343 | 8086370 |
| Probe end position | 8073383 | 8073743 | 8074103 | 8074463 | 8086490 |
| Recovery | 201978 | 369378 | 234772 | 317645 | 129583 |
| Sample 1 | | | | | |
| Recovery | 126320 | 207742 | 136406 | 168676 | 70739 |
| Sample 2 | | | | | |
| Recovery | 173782 | 297151 | 190790 | 221352 | 50497 |
| Sample 3 | | | | | |
| Recovery | 158386 | 243991 | 163333 | 217654 | 55265 |
| Sample 4 | | | | | |
| Recovery | 99214 | 187225 | 110764 | 140209 | 95508 |
| Sample 5 | | | | | |
| Recovery | 117951 | 207965 | 115162 | 161521 | 94527 |
| Sample 6 | | | | | |
| Recovery | 155741 | 257783 | 124676 | 193209 | 156337 |
| Sample 7 | | | | | |
| Recovery | 127108 | 226891 | 124620 | 166587 | 102286 |
| Sample 8 | | | | | |
| Recovery | 168822 | 316416 | 176456 | 249030 | 183054 |
| Sample 9 | | | | | |
| Recovery | 155337 | 240544 | 148619 | 226197 | 207253 |
| Sample 10 | | | | | |
| Recovery | 100245 | 168047 | 108343 | 134714 | 57988 |
| Sample 11 | | | | | |
| Recovery | 118261 | 221396 | 138920 | 166862 | 52964 |
| Sample 12 | | | | | |
| Recovery | 109532 | 215202 | 129918 | 159849 | 69206 |
| Sample 13 | | | | | |
| Recovery | 88490 | 154975 | 93200 | 115272 | 48649 |
| Sample 14 | | | | | |
| Recovery | 94106 | 183417 | 100504 | 138777 | 48451 |
| Sample 15 | | | | | |
| Recovery | 96044 | 187140 | 118314 | 145246 | 8701 |
| Sample 16 | | | | | |
| Recovery | 135034 | 231111 | 140979 | 178898 | 87149 |
| Sample 17 | | | | | |
| Recovery | 147402 | 228866 | 146385 | 179790 | 78745 |
| Sample 18 | | | | | |
| Recovery | 178507 | 273184 | 178672 | 227299 | 43104 |
| Sample 19 | | | | | |
| Recovery | 129231 | 244743 | 137377 | 184617 | 162741 |
| Sample 20 | | | | | |
| Average Recovery | 134075 | 233158 | 140911 | 184670 | 90137 |
| STDEV | 32270 | 51786 | 34273 | 47534 | 52422 |
| % CV | 24.1% | 22.2% | 24.3% | 25.7% | 58.2% |
| Avg. Normalized Recovery | 0.620 | 1.079 | 0.636 | 0.843 | 0.378 |
| Normalized STDEV | 0.045 | 0.064 | 0.042 | 0.064 | 0.099 |
| Normalized % CV | 7.2% | 6.0% | 6.6% | 7.6% | 26.3% |

TABLE 4
Recovery data for example probes in the first pool using post-deduplicated coverages.

| | Probe 1 | Probe 2 | Probe 3 | Probe 4 | Probe 5 |
|---|---|---|---|---|---|
| Probe start position | 8073263 | 8073623 | 8073983 | 8074343 | 8086370 |
| Probe end position | 8073383 | 8073743 | 8074103 | 8074463 | 8086490 |
| Recovery | 5646 | 7068 | 6990 | 6815 | 1744 |
| Sample 1 | | | | | |
| Recovery | 3187 | 3725 | 3545 | 3550 | 1045 |
| Sample 2 | | | | | |
| Recovery | 4768 | 5428 | 5450 | 5076 | 878 |
| Sample 3 | | | | | |
| Recovery | 3189 | 3560 | 3426 | 3610 | 610 |
| Sample 4 | | | | | |
| Recovery | 3162 | 3847 | 3737 | 3686 | 1382 |
| Sample 5 | | | | | |
| Recovery | 4791 | 5584 | 5228 | 5747 | 1800 |
| Sample 6 | | | | | |
| Recovery | 3957 | 4585 | 3734 | 4279 | 2162 |
| Sample 7 | | | | | |
| Recovery | 4703 | 5469 | 5348 | 5703 | 2029 |
| Sample 8 | | | | | |
| Recovery | 5613 | 6807 | 6051 | 6787 | 2693 |
| Sample 9 | | | | | |
| Recovery | 4717 | 4820 | 4668 | 5598 | 2633 |
| Sample 10 | | | | | |
| Recovery | 2943 | 3356 | 3390 | 3327 | 830 |
| Sample 11 | | | | | |
| Recovery | 3721 | 4769 | 4555 | 4464 | 862 |
| Sample 12 | | | | | |
| Recovery | 4361 | 5288 | 5120 | 5246 | 1125 |
| Sample 13 | | | | | |
| Recovery | 3325 | 3875 | 3651 | 3632 | 1005 |
| Sample 14 | | | | | |
| Recovery | 3891 | 4979 | 4827 | 4837 | 1123 |
| Sample 15 | | | | | |
| Recovery | 2413 | 3097 | 3080 | 2922 | 188 |
| Sample 16 | | | | | |
| Recovery | 3552 | 4133 | 4030 | 4084 | 1377 |
| Sample 17 | | | | | |
| Recovery | 3038 | 3492 | 3344 | 3246 | 891 |
| Sample 18 | | | | | |
| Recovery | 4998 | 5449 | 5612 | 5437 | 755 |
| Sample 19 | | | | | |
| Recovery | 5033 | 6433 | 5677 | 6087 | 3479 |
| Sample 20 | | | | | |
| Average Recovery | 4050 | 4788 | 4573 | 4707 | 1431 |
| STDEV | 946 | 1161 | 1090 | 1195 | 822 |
| % CV | 23.4% | 24.3% | 23.8% | 25.4% | 57.5% |
| Avg. Normalized Recovery | 0.908 | 1.068 | 1.000 | 1.059 | 0.286 |
| Normalized STDEV | 0.065 | 0.066 | 0.057 | 0.083 | 0.076 |
| Normalized % CV | 7.1% | 6.1% | 5.7% | 7.9% | 26.4% |

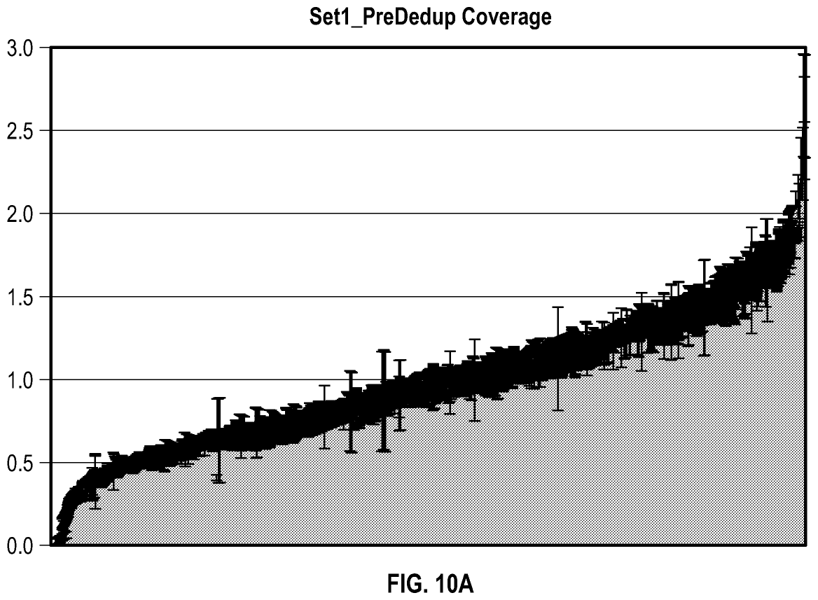

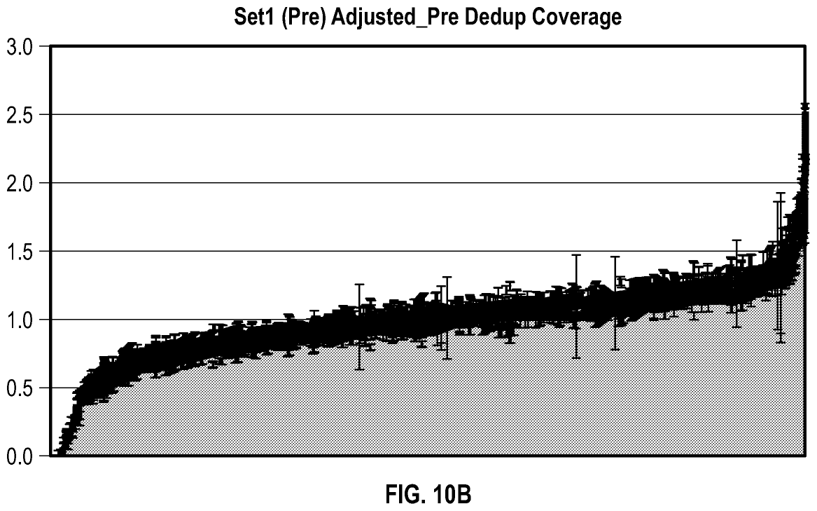

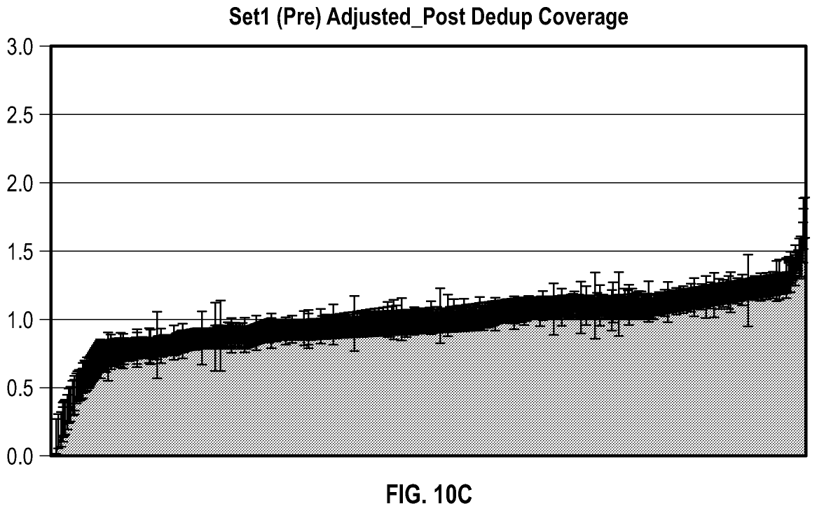

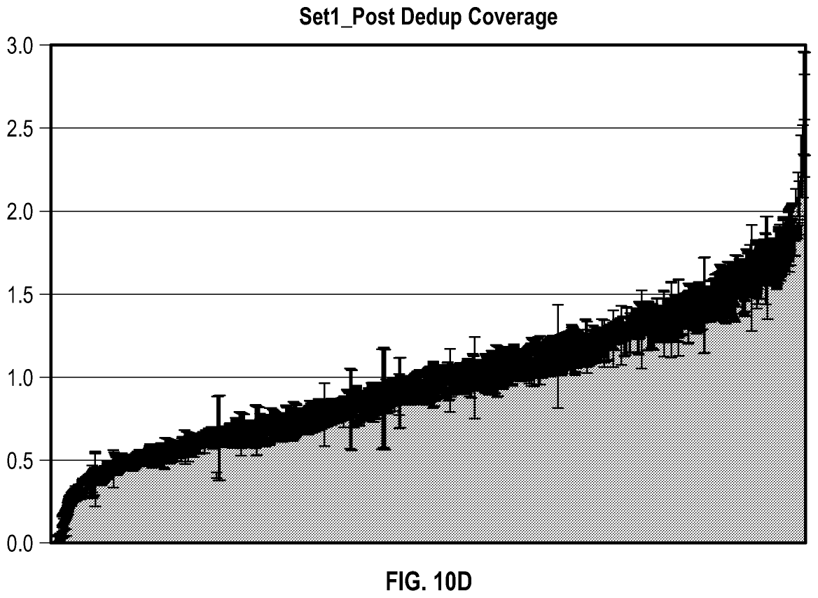

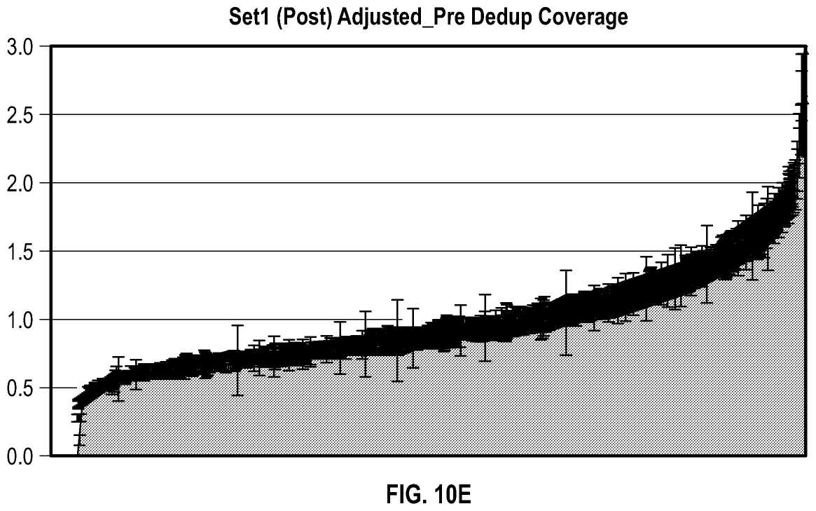

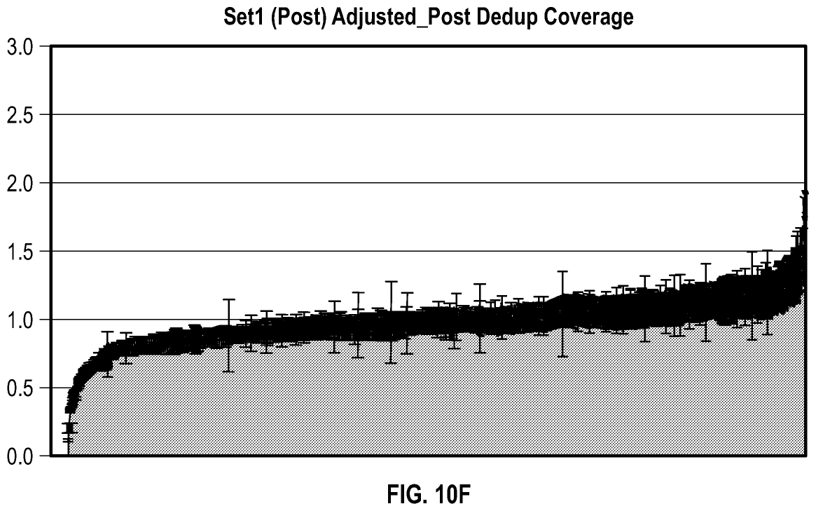

The average trimmed and normalized recoveries from the pre-deduplication and post-deduplication analyses were then ranked and plotted with error bars representing one standard deviation, as illustrated in FIGS. 10A and 10D, respectively. A target normalized recovery of 0.5, representing the 13^{th} percentile of average trimmed and normalized recoveries across the first pool, was selected. The level of biotinylation for each probe in the pool having an average trimmed and normalized recovery of greater than 0.5 was adjusted downward by a factor determined by dividing the average trimmed and normalized recovery rate by the target recovery rate, using the pre-deduplicated results and post-deduplicated results, to generate two second iterations of pool 1 for the probe set. For example, the normalized average recovery for Probe 1 in the post-deduplicated analysis is 0.908, as shown in Table 4. To determine the adjustment factor, 0.908 was divided by 0.5 giving a factor of 1.816. The biotinylation percentage of Probe 1, which was 100% in the first iteration of the pool, was adjusted downward by dividing 100% by 1.816, giving a second biotinylation percentage of 55.1% for the second iteration of the pool. That is, 55.1% of the copies of probe 1 in the second iteration of the pool were biotinylated and 44.9% were not biotinylated. The biotinylation percentage for probes with normalized average recoveries of less than 0.5 was not changed in the second iteration of the pool, i.e., was maintained at 100%. The adjusted biotinylation percentage for each of probes 1-5 in the second iterations of pool 1, as determined using pre-deduplicated data and post-deduplicated data, are shown in Tables 5 and 6, respectively.

TABLE 5
Adjusted biotinylation percentages for example
probes in the second instance of the first pool,
as adjusted using pre-deduplicated coverages.

| | Probe 1 | Probe 2 | Probe 3 | Probe 4 | Probe 5 |
|---|---|---|---|---|---|
| Avg. Normalized | 0.620 | 1.079 | 0.636 | 0.843 | 0.378 |
| Recovery | | | | | |
| Adjusted % | 19% | 54% | 21% | 41% | 0% |
| Unlabeled | | | | | |
| Adjusted % | 81% | 46% | 79% | 59% | 100% |
| Labeled | | | | | |

TABLE 6
Adjusted biotinylation percentages for example
probes in the second instance of the first pool,
as adjusted using post-deduplicated coverages.

| | Probe 1 | Probe 2 | Probe 3 | Probe 4 | Probe 5 |
|---|---|---|---|---|---|
| Avg. Normalized | 0.908 | 1.068 | 1.000 | 1.059 | 0.286 |
| Recovery | | | | | |
| Adjusted % | 45% | 53% | 50% | 53% | 0% |
| Unlabeled | | | | | |
| Adjusted % | 55% | 47% | 50% | 47% | 100% |
| Labeled | | | | | |

The second instances of the first pool, with adjusted biotinylation percentages for each probe species based on the pre-deduplicated or post-deduplicated analyses, were then used is a second round of experiments to enrich for nucleic acids in the twenty DNA libraries. The enriched nucleic acids were sequenced and sequence reads, either raw sequence reads or deduplicated sequences determined therefrom, were mapped to each probe. Recovery was then calculated for each probe in each sample based on pre-deduplicated sequence reads and deduplicated sequence reads and analyzed as described above for the first round of experiments. Summary statistics for the analysis of the five example probes, using pre-deduplicated data and post-deduplicated data are shown in Tables 7 and 8, respectively.

TABLE 7
Recovery data for example probes in the second instance of
the first pool adjusted using pre-deduplicated coverages.

| | | | | |
|---|---|---|---|---|
| Probe 1 | Probe 2 | Probe 3 | Probe 4 | Probe 5 |

Analysis based on normalized
pre-deduplicated recovery

| | | | | | |
|---|---|---|---|---|---|
| Avg. Normalized | 0.961 | 1.161 | 1.013 | 0.991 | 0.948 |
| Recovery | | | | | |
| Normalized STDEV | 0.074 | 0.074 | 0.093 | 0.096 | 0.311 |
| Normalized % CV | 7.7% | 6.4% | 9.2% | 9.7% | 32.7% |

Analysis based on normalized
post-deduplicated recovery

| | | | | | |
|---|---|---|---|---|---|
| Avg. Normalized | 0.926 | 1.119 | 1.048 | 1.009 | 0.420 |
| Recovery | | | | | |
| Normalized STDEV | 0.072 | 0.066 | 0.058 | 0.070 | 0.125 |
| Normalized % CV | 7.8% | 5.9% | 5.5% | 7.0% | 29.7% |

TABLE 8
Recovery data for example probes in the second instance of
the first pool adjusted using post-deduplicated coverages.

| | | | | |
|---|---|---|---|---|
| Probe 1 | Probe 2 | Probe 3 | Probe 4 | Probe 5 |

Analysis based on normalized
pre-deduplicated recovery

| | | | | | |
|---|---|---|---|---|---|
| Avg. Normalized | 0.961 | 1.161 | 1.013 | 0.991 | 0.948 |
| Recovery | | | | | |
| Normalized STDEV | 0.074 | 0.074 | 0.093 | 0.096 | 0.311 |
| Normalized % CV | 7.7% | 6.4% | 9.2% | 9.7% | 32.7% |

Analysis based on normalized
post-deduplicated recovery

| | | | | | |
|---|---|---|---|---|---|
| Avg. Normalized | 0.926 | 1.119 | 1.048 | 1.009 | 0.420 |
| Recovery | | | | | |
| Normalized STDEV | 0.072 | 0.066 | 0.058 | 0.070 | 0.125 |
| Normalized % CV | 7.8% | 5.9% | 5.5% | 7.0% | 29.7% |

The average trimmed and normalized recoveries from the pre-deduplication and post-deduplication analyses were then ranked and plotted with error bars representing one standard deviation, as illustrated in FIGS. 10B (normalized pre-deduplicated coverage) and 10C (normalized post-deduplicated coverage), for the probe set pool adjusted based on pre-deduplicated data, and FIGS. 10E (normalized pre-deduplicated coverage) and 10F (normalized post-deduplicated coverage), for the probe set pool adjusted based on post-deduplicated data, respectively.

As shown in FIGS. 10B, 10C, 10E, and 10F, and exemplified in the statistics shown in Tables 7 and 8, the second iteration of the probe pool resulted in a significantly more uniform recovery of target DNA than the original probe pool in which every probe species was 100% biotinylated. Significantly, recovery with even the under-performing probes sets, for which the biotinylation levels were unchanged in the second iteration, moved closer to the average recovery in the second iteration of the probe pool.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in FIG. 1, and/or as described in FIG. 2. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A method for forming an adjusted nucleic acid probe set comprising a plurality of nucleic acid probe species, comprising:
(A) obtaining a first iteration of a nucleic acid probe set, wherein the first iteration of the nucleic acid probe set comprises the plurality of nucleic acid probe species distributed in a first plurality of pools, wherein:
the plurality of nucleic acid probe species comprises, for each respective locus in at least a portion of a plurality of loci within a genome, a respective sub-plurality of nucleic acid probe species, wherein each respective nucleic acid probe species in the respective sub-plurality of nucleic acid probe species comprises a different subsequence, or the complement of the different subsequence, of the respective locus that is not present in the plurality of loci other than the respective locus,
each respective nucleic acid probe species in the plurality of nucleic acid probe species is present in the first iteration of the nucleic acid probe set in a combination of a respective first proportion and second proportion that sums to a respective amount, wherein each nucleic acid probe species in the respective first proportion is a non-nucleotidic capture moiety conjugated version of the respective nucleic acid probe species and each nucleic acid probe species in the respective second proportion, when the respective second proportion is other than zero, is a capture moiety-free version of the respective nucleic acid probe species,
each nucleic acid probe species present in a respective pool, in the first plurality of pools, aligns to a portion of the genome that is at least 50 nucleotides away from any other portion of the genome that any other nucleic acid probe species present in a respective pool aligns with;
(B) separately analyzing each respective pool in the first plurality of pools in the first iteration of the nucleic acid probe set against a first plurality of reference nucleic acid samples to obtain a corresponding first recovery rate of each respective nucleic acid probe species in the plurality of nucleic acid probe species;
(C) identifying, based on the corresponding first recovery rate of each respective nucleic acid probe species, a first subset of the plurality of nucleic acid probe species that does not satisfy a maximum recovery rate threshold; and
(D) reducing, for each respective nucleic acid probe species in the first subset of the plurality of nucleic acid probe species, the respective first proportion while maintaining the requirement that the respective first proportion and second proportion for the respective nucleic acid probe species sums to the respective amount, to form a first adjusted version of the nucleic acid probe set.

2-3. (canceled)

4. The method of claim 1, wherein, for the first iteration of the nucleic acid probe set, each nucleic acid probe species in the plurality of nucleic acid probe species is present in only one pool in the first plurality of pools.

5. The method of claim 1, wherein:
the first plurality of pools consists of three pools, and
each respective pool in the first plurality of pools consists of, for a respective locus in the plurality of genomic loci, every third respective nucleic acid probe species in the respective sub-plurality of nucleic acid probe species for the respective locus, such that the first plurality of pools collectively consists of each respective nucleic acid probe species in the respective sub-plurality of nucleic acid probe species for the respective locus.

6. The method of claim 1, wherein, in the first iteration of the probe set, the respective first proportion of each nucleic acid probe species in the plurality of nucleic acid probe species is the same.

7. (canceled)

8. The method of claim 1, wherein the molarity of each nucleic acid probe species in the plurality of nucleic acid probe species is the same for each nucleic acid probe species in the plurality of nucleic acid probe species in the first iteration of the probe set.

9. The method of claim 8, wherein the molarity of each nucleic acid probe species in the plurality of nucleic acid probe species is the same in a second iteration of the probe set that is based on the first adjusted version of the nucleic acid probe set.

10. (canceled)

11. The method of claim 1, wherein the non-nucleotidic capture moiety is biotin, biotin carbonate 5, biotin carbamate 6, Iminobiotin, or Desthiobiotin.

12. The method of claim 1, wherein the different subsequence of the respective nucleic acid probe species in the respective sub-plurality of nucleic acid probe species is from 75 nucleotides to 250 nucleotides in length.

13. The method of claim 1, wherein the respective sub-plurality of nucleic acid probe species for a respective locus in the at least the portion of the plurality of loci consists of non-overlapping nucleic acid probe sequences.

14. The method of claim 13, wherein a gap between any two different subsequences within the respective locus, from any pair of respective nucleic acid probe species in the respective sub-plurality of probe species, is no more than 10 nucleotides.

15. The method of claim 1, wherein the sub-plurality of nucleic acid probe species for a respective locus in the plurality of loci consists of overlapping nucleic acid probe sequences.

16. (canceled)

17. The method of claim 1, wherein the analyzing (B) comprises, for each respective pool in the first plurality of pools:
capturing, for each respective reference nucleic acid sample in the plurality of reference samples, nucleic acids from the respective reference nucleic acid sample using the respective pool;
measuring, for each respective nucleic acid probe species present in the respective pool, a respective first recovery rate for each respective reference nucleic acid sample in the plurality of nucleic acid reference samples, thereby obtaining a corresponding plurality of respective first recovery rates for the respective nucleic acid probe species; and
determining, for each respective nucleic acid probe species present in the respective pool, the corresponding first recovery rate for the respective nucleic acid probe species based on the corresponding plurality of respective first recovery rates.

18-21. (canceled)

22. The method of claim 17, wherein the first recovery rate of a respective nucleic acid probe species is determined by sequencing the captured or amplified nucleic acids, de-duplicating raw sequence reads from the sequencing to generate unique sequence reads, and quantitating the number of unique sequence reads that overlap the respective nucleic acid probe by a minimum number of nucleic acids.

23. (canceled)

24. The method of claim 1, wherein the maximum recovery threshold is set relative to a measure of central tendency for the first recovery rate across all of the nucleic acid probe species.

25. The method of claim 24, wherein the maximum recovery threshold is no more than 10% greater than the measure of central tendency for the first recovery rate across all of the nucleic acid probe species.

26. The method of claim 1, wherein, in the first adjusted version of the nucleic acid probe set:
the respective first proportion of each respective nucleic acid probe species in the first subset of the plurality of nucleic acid probe species is at a lower proportion than the respective first proportion of each respective nucleic acid probe species in the plurality of nucleic acid probe species that satisfied the maximum recovery rate threshold.

27. The method of claim 1, wherein each respective first proportion of each respective nucleic acid probe species in the first subset of the plurality of nucleic acid probe species is reduced by an amount that is proportional to a difference between the corresponding first recovery rate for the respective nucleic acid probe and a maximum reference recovery rate.

28. The method of claim 27, wherein the maximum reference recovery rate is the maximum recovery rate threshold.

29. The method of claim 27, wherein the maximum reference recovery rate is set relative to the first recovery rate across all of the nucleic acid probe species present in the respective pool.

30. The method of claim 29, wherein the maximum reference recovery rate is set to a value equal to a percentile first recovery rate across all of the nucleic acid probe species present in the respective pool.

31. (canceled)

32. The method of claim 1, further comprising:
identifying, based on the corresponding first recovery rate of each respective nucleic acid probe species, a second subset of the plurality of nucleic acid probe species that does not satisfy a minimum recovery rate threshold; and
increasing, for each respective nucleic acid probe species in the second subset of the plurality of probe species, the respective first proportion while maintaining the requirement that the respective first proportion and second proportion for the respective nucleic acid probe species sums to the respective amount.

33. The method of claim 32, further comprising increasing, for each respective nucleic acid probe species in the second subset of the plurality of nucleic acid probe species, the respective first proportion proportional to a difference between the corresponding first recovery rate for the respective nucleic acid probe and a minimum reference recovery rate while maintaining the requirement that the respective first proportion and second proportion for the respective nucleic acid probe species sums to the respective amount.

34. The method of claim 1, further comprising:
(E) obtaining a second iteration of the nucleic acid probe set, wherein the second iteration of the nucleic acid probe set comprises the plurality of nucleic acid probe species distributed in a second plurality of pools, wherein:
each respective nucleic acid probe species in the plurality of probe species is present in the second iteration of the nucleic acid probe set in a combination of a respective third proportion and fourth proportion that sums to a respective amount, wherein each nucleic acid probe species in the respective third proportion is a non-nucleotidic capture moiety conjugated version of the respective nucleic acid probe species and each nucleic acid probe species in the respective fourth proportion is a capture moiety-free version of the respective nucleic acid probe species, based on the first adjusted version of the final design for the nucleic acid probe set, and
each nucleic acid probe species present in a respective pool, in the second plurality of pools, aligns to a portion of the genome that is at least 100 nucleotides away from any other portion of the genome that any other nucleic acid probe species present in a respective pool aligns with;
(F) separately analyzing each respective pool in the second plurality of pools in the second iteration of the nucleic acid probe set against a second plurality of reference nucleic acid samples, in which a relative amount of representation of each locus in the plurality of loci by the second plurality of reference nucleic acid samples is known, to obtain a corresponding second recovery rate of each respective nucleic acid probe species in the plurality of nucleic acid probe species;
(G) identifying, based on the corresponding second recovery rate of each respective nucleic acid probe species, a third subset of the plurality of nucleic acid probe species that does not satisfy a maximum recovery rate threshold; and
(H) reducing, for each respective nucleic acid probe species in the third subset of the plurality of nucleic acid probe species, the respective second proportion while maintaining the requirement that the respective first proportion and second proportion for the respective nucleic acid probe species sums to the respective amount to form a second adjusted version of the nucleic acid probe set.

35. The method of claim 34, further comprising:
identifying, based on the corresponding second recovery rate of each respective nucleic acid probe species, a fourth subset of the plurality of nucleic acid probe species that does not satisfy a minimum recovery rate threshold; and
increasing each respective second proportion of each respective nucleic acid probe species in the fourth subset of the plurality of nucleic acid probe species.

36. (canceled)

37. The method of claim 34, wherein the analyzing (F) comprises, for each respective pool in the second plurality of pools:
capturing, for each respective reference nucleic acid sample in the plurality of reference samples, nucleic acids from the respective reference nucleic acid sample using the respective pool;
measuring, for each respective nucleic acid probe species present in the respective pool, a respective second recovery rate for each respective reference nucleic acid sample in the plurality of nucleic acid reference samples, thereby obtaining a corresponding plurality of respective second recovery rates for the respective nucleic acid probe species; and
determining, for each respective nucleic acid probe species present in the respective pool, the corresponding second recovery rate for the respective nucleic acid probe species based on the corresponding plurality of respective second recovery rates.

38-41. (canceled)
